(19) United States
(12) Patent Application Publication
(10) Pub. No.: US 2008/0069857 A1
(43) Pub. Date: Mar. 20, 2008

(54) Compositions And Methods For Inhibiting Adhesions

(76) Inventors: Yoon Yeo, Lafayette, IN (US); Taichi Ito, Aoba (JP); Robert S. Langer, Newton, MA (US); Daniel S. Kohane, Newton, MA (US); George Kevork Kodokian, Kennett Square, PA (US)

Correspondence Address: CHOATE, HALL & STEWART LLP, TWO INTERNATIONAL PLACE, BOSTON, MA, 02110, US

(21) Appl. No.: 11/734,537

(22) Filed: Apr. 12, 2007

Related U.S. Application Data
(60) Provisional application No. 60/791,362, filed on Apr. 12, 2006.
(60) Provisional application No. 60/857,557, filed on Nov. 8, 2006.
(60) Provisional application No. 60/901,241, filed on Feb. 13, 2007.

(51) Int. Cl. A61K 9/14; A61K 31/717; A61P 43/00; A61K 31/721

(52) U.S. Cl. 424/426; 424/489; 424/501; 514/57; 514/58

(57) ABSTRACT

The present invention provides compositions and methods for inhibiting adhesions. The methods involve administering solutions containing hydrogel precursors such as polysaccharide derivatives, e.g., derivatives of hyaluronic acid, cellulose, or dextran, to a subject at a site where adhesions may form, e.g., as a consequence of surgery, injury, or infection. The hydrogel precursors, e.g., polysaccharide derivatives, become crosslinked following their administration to form a hydrogel that maintains tissue separation. In certain embodiments of the invention one or both solutions contains particles, e.g., polymeric nanoparticles or microparticles, so that a composite hydrogel containing the particles is formed. The solution(s), particle(s), or both, may contain a biologically active agent such as an agent that contributes to inhibiting adhesions. The biologically active agent may be covalently attached to a hydrogel precursor.

### RELATED APPLICATIONS

The present application claims priority under 35 U.S.C. §119(e) to U.S. provisional patent applications U.S. Ser. No. 60/791,362, filed Apr. 12, 2006, U.S. Ser. No. 60/857,557, filed Nov. 8, 2006, and U.S. Ser. No. 60/901,241, filed Feb. 13, 2007, all of which are incorporated herein by reference.

### GOVERNMENT SUPPORT

The work described herein was supported, in part, by grants from the National Institutes of Health (GM073626). The United States government may have certain rights in the invention.

### BACKGROUND OF THE INVENTION

Adhesions are attachments between tissues, organs, or other anatomical structures that are normally separate from one another. They are typically composed of fibrous bands of scar-like tissue and often arise following a stimulus such as surgery, injury, or infection. Post-operative adhesions are a common and potentially serious occurrence as they can entail severe complications such as abdominal and pelvic pain, infertility, and bowel obstruction. It is estimated that 80% of abdominal surgeries result in adhesions, leading to an enormous cost in terms of human suffering and financial expense. Adhesions that form after surgery in the pelvic area are among the leading causes of post-operative pelvic pain, infertility, and small bowel obstruction. Trauma and infections, particularly in the abdominal or pelvic regions, can also result in adhesions.

Numerous pharmacological and barrier-based approaches to preventing or treating adhesions have been tested, at least in animal models, and several of the latter are in commercial use. These include such products as Interceed (TC7) Absorbable Adhesion Barrier (Johnson & Johnson) and Seprafilm® membrane (Genzyme Corp.) Many of the existing barrier devices are composed at least in part of crosslinked polysaccharides or glycosaminoglycans.

Hyaluronic acid (HA) has received considerable attention as a material for these purposes (Burns et al. Prevention of tissue injury and postsurgical adhesions by precoating tissues with hyaluronic acid solutions. Journal of Surgical Research 1995;59:644-652; Peck et al. Polymer solutions and films as tissue-protective and barrier adjuvants. In: diZerega G S, editor. Peritoneal Surgery. New York: Springer, 2000. p. 499-520; and Rodgers et al. Reproduction of adhesion formation with hyaluronic acid after peritoneal surgery in rabbits. Fertil. Steril. 1997;67(3):553-558, each of which is incorporated herein by reference.) HA is a linear polysaccharide composed of β-1,4-linked D-glucuronic acid (GlcUA) and β-1,3 N-acetyl-D-glucosamine (GlcNAc) disaccharide units and is a ubiquitous component of mammalian extracellular matrix. In its native form, HA is biocompatible, biodegradable, and relatively non-immunogenic. Notwithstanding these desirable properties, the existing HA-based approaches for inhibiting de novo or recurrent post-operative adhesions suffer from significant drawbacks. For example, when applied as a solution, the effectiveness of HA has been compromised by rapid clearance from the peritoneal cavity (Sawhney et al. Optimization of photopolymerized bioerodible hydrogel properties for adhesion prevention. J. Biomed. Mater. Res. 1994;28:831-838, which is incorporated herein by reference). Solid formulations of HA either alone or in combination with other materials, e.g., in the form of prepared sheets, have limitations including difficulty in applying the film (e.g., difficulty in handling, adherence of dried film to gloves, insufficient pliability, need for removal), incompatibility with laparoscopic procedures, and a lower than desirable efficacy. Thus there remains a need in the art for improved compositions and methods for inhibiting adhesions.

### SUMMARY OF THE INVENTION

The present invention provides compositions and methods for inhibiting adhesions. While the compositions and methods may be used for inhibiting formation, progression, or recurrence of adhesions at any location in the body, it is contemplated that they will find particular use for inhibiting peritoneal adhesions.

It is an object of the present invention to provide polysaccharide derivatives, e.g., HA, cellulose, or dextran derivatives, suitable for in situ polymerization within the body that are useful to inhibit the development, progression, and/or recurrence of adhesions. It is a further object of the invention to provide methods for inhibiting the formation, progression, and/or recurrence of adhesions by the administration of polysaccharide derivatives that cross-link rapidly to one another in situ following application to a site of tissue damage or injury, thereby forming a hydrogel that inhibits the formation, progression, and/or recurrence of adhesions.

It is also an object of the invention to provide polysaccharide derivatives and combinations thereof that undergo rapid crosslinking and gelation in a time frame that is advantageous for their application in situ. It is also an object of the invention to provide methods for producing hydrogels formed from crosslinked polysaccharides that afford control over parameters such as gelation time and half-life.

It is also an object of this invention to provide hybrid polysaccharide-based hydrogel compositions, wherein the compositions provide delivery of biologically active agents, e.g., therapeutic agents, to the body, optionally in a sustained manner. In certain embodiments, the biologically active agent is an anti-inflammatory agent, for example, glucocorticoids (e.g., prednisone, dexamethasone, budesonide), and non-steroidal anti-inflammatory agents (e.g., ibuprofen, aspirin). In certain embodiments, the biologically active agent is a fibrinolytic agent such as a plasminogen activator or streptokinase. These agents may optionally be incorporated into polymeric materials or matrices for extended or controlled release of the agent. In certain embodiments, the agent is conjugated directly to the hydrogel or one of the hydrogel precursors.

It is another object of the invention to provide hydrogel compositions containing particles that optionally deliver a biologically active agent to the body. In one aspect, the invention provides a method of inhibiting adhesions comprising the step of: administering a first hydrogel precursor and a second hydrogel precursor to a location within the body of a subject; wherein the first and second hydrogel precursors become crosslinked to form a hydrogel following contact with one another, and wherein the hydrogel inhibits adhesions. The first and second hydrogel precursors may be provided in one or more solutions. In certain embodiments of the invention the hydrogel precursors are polysaccharide derivatives. In one aspect, the invention provides a method of inhibiting adhesions comprising the step of: administering a first polysaccharide derivative to a location within the body of a subject; and administering a second polysaccharide derivative to the location within the body of the subject, wherein the first and second polysaccharide derivatives become crosslinked to form a hydrogel following contact of the polysaccharide derivatives with one another, and wherein the hydrogel inhibits adhesions. In certain embodiments of the invention the first polysaccharide derivative comprises a first functional group and the second polysaccharide derivative comprises a second functional group, and the first and second functional groups react with one another to form a covalent bond under physiological conditions. In certain embodiments of the invention one of the functional groups is a hydrazide and one of the functional groups is an aldehyde. In certain embodiments of the invention the polysaccharide derivatives are HA derivatives. In certain embodiments of the invention one of the polysaccharide derivatives is an HA derivative and the other polysaccharide derivative is a cellulose derivative (e.g., carboxymethylcellulose (CMC), hydroxypropylmethyl cellulose (HPMC), methyl cellulose (MC)). In certain other embodiments of the invention, one of the polysaccharide derivatives is an HA derivative and the other polysaccharide derivative is a dextran derivative. In certain embodiments the invention comprises administering a solution comprising a first polysaccharide derivative to a location within the body of a subject; and administering a second solution comprising a second polysaccharide derivative to the location within the body of the subject

In certain embodiments of the invention at least one of the polysaccharide derivatives comprises a non-polysaccharide portion. In other embodiments at least one of the hydrogel precursors is a non-polysaccharide polymer.

In another aspect, the invention provides a composition comprising a hyaluronic acid (HA) derivative in solution, wherein the concentration of the HA derivative is greater than 5 mg/ml. In another embodiment, the invention provides a composition comprising a hyaluronic acid (HA) derivative in solution, wherein the concentration of the HA derivative is greater than 10 mg/ml. In another embodiment, the invention provides a composition comprising a hyaluronic acid (HA) derivative in solution, wherein the concentration of the HA derivative is greater than 15 mg/ml. In another embodiment, the invention provides a composition comprising a hyaluronic acid (HA) derivative in solution, wherein the concentration of the HA derivative is greater than 25 mg/ml. In certain embodiments of the invention the concentration of the HA derivative is less than or equal to 100 mg/ml. In other embodiments of the invention the concentration of the HA derivative is between 50 mg/ml and 75 mg/ml.

In another aspect, the invention provides a hydrogel comprising crosslinked HA derivatives, wherein the hydrogel has a half-life of at least 10 days in the presence of 10 U/ml hyaluronidase. In certain embodiments, the invention provides an HA-cellulose, HA-dextran, or HA-other polysaccharide derivative, wherein the resulting hydrogel is less susceptible to hyaluronidase than the corresponding HA-HA hydrogel. In certain embodiments, the invention provides a composition comprising a cellulose or dextran derivative in solution, wherein the concentration of the cellulose or dextran derivative is greater than 5 mg/ml, greater than 10 mg/ml, greater than 15 mg/ml, or greater than 25 mg/ml.

In another aspect, the invention provides a composition comprising a first polysaccharide derivative; and a plurality of particles. The polysaccharide derivative may be an HA derivative, a cellulose derivative, or a dextran derivative.

In another aspect, the invention provides a composition comprising first and second polysaccharide derivatives; and a plurality of particles. In certain embodiments of the invention, the first and second polysaccharide derivatives are crosslinked to form a hydrogel. The particles may contain a biologically active agent. In certain embodiments, the biologically active agent is an anti-inflammatory agent (e.g., dexamethasone, prednisone, budesonide, ibuprofen, aspirin, etc.). In other embodiments, the biologically active agent is a fibrinolytic agent (e.g., a plasminogen activator, streptokinase).

The invention further provides a method of inhibiting adhesions comprising the step of: administering a plurality of particles and at least one polysaccharide derivative to a location within the body of a subject wherein the first polysaccharide derivative either alone or in combination with a second polysaccharide derivative becomes crosslinked to form a hydrogel that entraps the particles after administration. In certain embodiments of the invention the method comprises administering first and second polysaccharide derivatives, wherein at least one derivative is an HA derivative. In certain embodiments of the invention, at least one derivative is a cellulose derivative. In certain embodiments of the invention, at least one derivative is a dextran derivative. In certain embodiments of the invention, at least one derivative comprises a non-polysaccharide portion.

The invention further provides a method of inhibiting adhesions comprising the step of: administering a first solution comprising a first polysaccharide derivative to a location within the body of a subject; and administering a second solution comprising a second polysaccharide derivative to the location, wherein either or both of the solutions comprises a plurality of particles, and wherein the polysaccharide derivatives become crosslinked to form a hydrogel that entraps the particles after administration. In certain embodiments of the invention the first solution comprises a first HA derivative and the second solution comprises a second HA derivative. In certain embodiments of the invention the first solution comprises an HA derivative and the second solution comprises a cellulose derivative. In certain embodiments of the invention, the first solution comprises an HA derivative and the second solution comprises a dextran derivative. In certain embodiments of the invention, the first solution comprises an HA derivative and the second solution comprises another polysaccharide derivative.

In another aspect the invention provides a method of administering particles to a location within the body: comprising administering a composition comprising particles and one or more hydrogel precursors to the location, wherein the one or more hydrogel precursors form a hydrogel that entraps the particles therein. In certain embodiments of the invention at least one of the hydrogel precursors is a polysaccharide derivative.

### BRIEF DESCRIPTION OF THE DRAWING

FIG. 1. Scanning electron micrographs of a HAX gel. Scale bar=10 μm.

FIG. 2. Viability of mesothelial cells in the presence of HAX gels (20 mg/ml). White bars and gray bars indicate cells grown in plain medium and in medium containing 10 U/ml hyaluronidase, respectively. Data are medians with 25th and 75th percentiles (n=4).

FIG. 3. (A) HAX applied on the injured abdominal wall and cecum. Arrows indicate gels adherent to the applied sites. (B) no adhesion observed in an animal treated with HAX (C) score 3 adhesion observed in no treatment control. AW=abdominal wall.

FIG. 4. Histological examination. (A) Adhesion from an untreated animal (5×). Arrows indicate injured muscle cells; (B) close-up image of adhesion from another untreated animal (10×). Note the bands of high cell population indicating inflammation and fibrosis. (C) Adhesion free abdominal wall from an animal treated with 20 mg/ml HAX (20×). Note the bluish coating on the lumen side. AW=abdominal wall muscle, Sm=smooth muscle.

FIG. 5. (A) Glucuronic acid release during the degradation of HAX gels in 10 U/ml hyaluronidase (n=5). (B) tPA production by mesothelial cells in the presence of hyaluronic acid (HA) and its monomer components.

FIG. 6. Effects of (A) concentration and (C) Mw of HA-CHO on macroscopic gel degradation kinetics. Legends indicate Mws and concentrations of HA-ADH or HA-CHO in mg/ml. Values are indicated as means and standard deviations of four measurements.

FIG. 7. SEM pictures of (A) PLGA nanoparticles, (B) lyophilized HAX gel, and (C) lyophilized hybrid gel.

FIG. 8. Viability of mesothelial cells in the presence of hybrid gels (20 mg/ml HAX, PLGA nanoparticles). White bars and gray bars indicate cells grown in plain media and in media containing 10 U/ml hyaluronidase, respectively. Data are medians with 25th and 75th percentiles (n=4).

FIG. 9. Hybrid gels (indicated by arrows) remaining in the peritoneum 2 days or 7 days after injection. (A) 10 mg/ml HAX+20 mg/ml PLGA nanoparticles (B and C) 20 mg/ml HAX+20 mg/ml PLGA. The inset in (A) shows a hybrid gel separated from the peritoneum. B=bowel, AW=abdominal wall.

FIG. 10. (A) Hybrid gel applied on the injured abdominal wall and cecum. Arrows indicate hybrid gels adherent to the applied sites. (B) No adhesion observed in an animal treated with Hybrid gel. AW=abdominal wall.

FIG. 11. Histological examination. (A) Hybrid gel recovered from a rabbit 7 days post-surgery (200×). Note foamy macrophages. (B) Close-up image of foamy macrophages (400×) similar to that in a gel residue found in a mouse (inset) 7 days after injection. (C) Adhesion free abdominal muscle wall from a rabbit treated with hybrid gel (200×); (D) close-up image of abdominal muscle wall surface (400×) that was covered with hybrid gel during the surgery. Note the foaminess of the macrophages. AW=abraded abdominal wall muscle.

FIG. 12 shows the chemical structures of various synthesized polysaccharide derivatives. (A)HA-ADH; (B) HA-ALD; (C)CMC-ALD (R═CH₂COOH or H), HPMC-ALD (R═CH₂CH(OH)CH₃ or H), or MC-ALD (R═CH₃ or H).

FIG. 13 shows a device useful for administering solutions containing crosslinkable polysaccharide derivatives.

FIG. 14 shows a multi-channel device useful for administering solutions containing crosslinkable polysaccharide derivatives.

FIG. 15 shows a multi-barrel device useful for administering solutions containing crosslinkable polysaccharide derivatives.

FIG. 16 is a bar graph that shows the ability of a variety of hydrogels formed by crosslinking an HA derivative and a cellulose derivative to inhibit adhesions.

FIG. 17. Degradation kinetics of the hydrogels in 10 unit/ml hyaluronidase in PBS at 37° C. Volume of the hydrogel (%) is the ratio of the volume of hydrogel at each time point to the initial volume, expressed as a percentage. Data are averages±standard deviations (n=4).

FIG. 18. Effect of aldehyde polymers (HA-CHO, CMC—CHO, MC—CHO, and HPMC—CHO) on cell viability measured by the MTT assay. (A) Mesothelial cells after 3 days incubation with polymers. (B) Macrophages (J774.A1 cell line), after 2 days incubation with polymers. Data are averages±standard deviations (n=4).

FIG. 19. Peritoneums of mice 1 week after injection of hydrogels. (A) HAX: no residue. (B) HA-CMC: note the thin coating of gel-like material. (C) HA-MC: note the increased amount of residual material, demonstrated the forceps submerged beneath it.

FIG. 20. Prevention of peritoneal adhesions in a rabbit abrasion model. (A) Induction of adhesions. Note the abdominal wall defect (arrow), and the bleeding surface of the cecum. (B) Adhesions seen on dissection after 1 week in an animal treated with saline. (C) Absence of adhesions after 1 week in an animal treated with HA-MC.

FIG. 21. Photomicrographs of tissues recovered 1 week after injury in the rabbit sidewall defect-bowel abrasion model. (A) Cross-section of an abrasion in a saline-treated animal. The cecal lumen (CE) is in the left upper corner of the picture. The cecal smooth muscle is fused to the striated muscle of abdominal musculature (AM). Magnification 100×. (B) Hydrogel recovered from an animal treated with HA-MC, with inflammatory cells (predominantly macrophages and lymphocytes). Magnification 100×. (C) Site of abdominal wall defect in an animal treated with HA-MC. The defect has been re-epithelialized (arrows), with a subjacent layer of healing tissue (predominantly fibroblasts). Magnification 400×. (D) Normal untreated parietal peritoneum. The mesothelium (arrows) overlies connective tissue (CT) and abdominal muscle.

FIG. 22. Chemical structure of (A) DX; (B) DX—CHO; (C) CMDX; (D) CMDX-ADH; (E) CMC; and (F) CMC-CHO.

FIG. 23. FT-IR spectra of (A) DX; (B) CMD; and (C) CMD-ADH.

FIG. 24. Swelling volumes of the hydrogels in PBS buffer at 37° C. The measured values are average±standard deviation (N=4).

FIG. 25. Gelation time of the hydrogels. The measured values are average±standard deviation (N=5).

FIG. 26. Swelling volumes of the hydrogels in PBS buffer at 37° C. The measured values are average±standard deviation (N=4).

FIG. 27. Pictures of the hydrogels 5 days after immersing in PBS buffer at 37° C. (A) 70 kDa-CMDX-DX (5% (w/v)/6% (w/v)). (B) 70 kDa-CMDX—CMC (5% (w/v)/6% (w/v)).

FIG. 28. Effect of the unmodified and synthesized polymers (DX, CMC, CMDX-ADH, CMC—CHO, and DX—CHO) on cell viability measured by MTT assay. Data are average±standard deviation (N=4). (A) Mesothelial cells after 3 days incubation with polymers. The data of CMC—CHO is described above in Example 12. (B) J774.A1, macrophages cell line, after 2 days incubation with polymers.

FIG. 29. Peritoneum of mice 2 weeks after injection of 70 kDa-CMDX-DX.

FIG. 30. Peritoneal of adhesion preventing tests by a rabbit abrasion model. Laparotomies were performed one week after the adhesion-inducing surgery. (A-1, A-2) 70 kDa-CMDX-DX (2% (w/v)/5% (w/v)). CMDX-DX gels made peritoneal adhesions worse. (B) 70 kDa-CMDX—CMC (5% (w/v)/6% (w/v)) (C-1,C-2) 500 kDa-CMDX—CMC (4% (w/v)/6% (w/v)) CMDX—CMC gels reduced the peritoneal adhesions. The result of control experiments was cited from our previous study.

FIG. 31. Histology pictures of a rabbit sidewall defect-bowel abrasion model experiments. (A) CMDX-DX gel stuck on the haustra 14 of cecum (5×). (B) The magnification of the sticking surface of panel A (40×). (C) Recovered abdominal wall in the case of CMDX-DX gel (5×). (D) Normal abdominal wall in 500 kDa-CMDX—CMC gel (5×).

FIG. 32. Schematic of the synthesis of the cross-linked hyaluronic acid hydrogel containing dexamethasone (HAX-DEX). The final hydrogel is formed by mixing the aldehyde-derivatized hyaluronic acid with hyaluronic acid-adipic dihydrazide-dexamethasone succinate (shaded compounds).

FIG. 33. Viability of human mesothelial cells incubated with different concentration of synthesized polymers, determined by MTT assay. Data are means with standard deviations.

FIG. 34. Time course of the concentration of dexamethasone in the released media. Data are means with standard deviations.

FIG. 35. Time course of the swelling volume of HAX and HAX-DEX. Data are means with standard deviations.

FIG. 36. The effect of dexamethasone on the production of TNF-α and IL-6 from primary mouse macrophages. Values are the average of two measurements.

FIG. 37. Time course of the production of IL-6 from primary mouse macrophages. Data are means with standard deviations.

FIG. 38. Time course of the production of TNF-α from primary mouse macrophages. Data are means with standard deviations.

FIG. 39. Hydrogels removed 2 days after injection. (A) HAX-DEX hydrogel ex vivo. (B) Representative hematoxylin-eosin stained sample of HAX-DEX gel (top) with subjacent muscle (bottom), 20×. (C and D) Two examples of inflammatory cells in and surrounding HAX gels, 20× and 40× respectively. The blue relatively homogeneous material is the gel (G).

FIG. 40. Scanning electron micrograph of lyophilized budesonide-HAX. Scale bar=100 μm.

FIG. 41. Solubility of budesonide in saline at 37° C. (A) Phase-solubility diagram. (B) Budesonide concentration and mass of precipitates over time. Data are averages and standard deviations (n=4).

FIG. 42. In vitro release kinetics of budesonide from budesonide-HAX. Data are averages and standard deviations (n=4).

FIG. 43. (A) Weight loss (as percentage of starting body mass) after the second laparotomy. (B) percentage of animals with each adhesion score. Score 0=no adhesion, score 2=tissue adhesion separable by blunt dissection, score 3=adhesion requiring sharp dissection. (C) sum of areas with score 2 and 3 adhesions (cm²). Weight loss and adhesion areas are expressed as medians with 25 and 75 percentiles (n=6). *indicates statistical difference from saline control, and † and ‡ indicate statistical difference between the compared groups. Saline-treated control and HAX groups are from ref [tPA].

FIG. 44. Tissues from an animal treated with budesonide-saline and normal tissues. (A) Abdominal wall surface (200×); (B) cecum surface (200×); and (C) cecum surface (400×). SK: abdominal wall skeletal muscle; SM: visceral smooth muscle; Me: mesothelial layer.

FIG. 45. Hydrogels removed 2 days after injection. (A-D) Gross appearance of dissection. (A) HAX in situ. Note the inflammation and marked vascularity. (B) Budesonide-HAX in situ. (C) HAX ex vivo, inseparable from skin. (D) Budesonide-HAX, separated from skin except for a small rind left intentionally. (E and F) Hematoxylin-eosin stained sections (both 40×). (E) HAX. Note the massive inflammatory reaction. (F) Budesonide-HAX. Note the relative absence of inflammation. In E and F, the pale bluish material in the lumen is the hydrogel, surrounded by an esosiophilic capsule.

### DEFINITIONS

The terms “angiogenesis inhibitor” and “anti-angiogenic agent” are used interchangeably herein to refer to agents that are capable of inhibiting or reducing one or more processes associated with angiogenesis including, but not limited to, endothelial cell proliferation, endothelial cell survival, endothelial cell migration, differentiation of precursor cells into endothelial cells, and capillary tube formation.

“Anti-infective agent,” as used herein, refers to any substance that inhibits the proliferation of one or more infectious agents, e.g., virus, bacteria, fungus, protozoa, helminth, fluke, or other parasite. The anti-infective agent may display inhibitory activity in vitro (i.e., in cell culture), in vivo (i.e., when administered to an animal at risk of or suffering from an infection), or both. Preferably the anti-infective agent has inhibitory activity in vivo at therapeutically tolerated doses.

“Anti-inflammatory agent,” as used herein, refers to any substance that inhibits one or more signs or symptoms of inflammation.

An “aqueous medium” as used herein means a liquid medium containing water and, optionally, one or more water-miscible solvents (e.g., dimethylformamide, dimethylsulfoxide, and hydrocarbyl alcohols, diols, or glycerols). An aqueous medium may contain at least 50%, 60%, 70%, 80%, 90% or more water by volume. It will be appreciated that an aqueous medium may contain a variety of substances dissolved, dispersed, or suspended therin.

The term “approximately” in reference to a number generally includes numbers that fall within a range of 5% in either direction of the number (greater than or less than the number) unless otherwise stated or otherwise evident from the context (except where such number would exceed 100% of a possible value).

“Biocompatible” refers to a material that is substantially nontoxic to a recipient's cells in the quantities and at the location used, and also does not elicit or cause a significant deleterious or untoward effect on the recipient's body at the location used, e.g., an unacceptable immunological or inflammatory reaction, unacceptable scar tissue formation, etc.

“Biodegradable” means that a material is capable of being broken down physically and/or chemically within cells or within the body of a subject, e.g., by hydrolysis under physiological conditions and/or by natural biological processes such as the action of enzymes present within cells or within the body, and/or by processes such as dissolution, dispersion, etc., to form smaller chemical species which can typically be metabolized and, optionally, used by the body, and/or excreted or otherwise disposed of. Preferably a biodegradable compound is biocompatible. For purposes of the present invention, a polymer whose molecular weight decreases over time in vivo due to a reduction in the number of monomers is considered biodegradable.

A “biologically active agent” is any compound or agent, or its pharmaceutically acceptable salt, which possesses a desired biological activity, for example therapeutic, diagnostic and/or prophylactic properties in vivo. It is to be understood that the agent may need to be released from particles and/or from a hydrogel in order for it to exert a biological activity. Biologically active agents include, but are not limited to, therapeutic agents as described herein. Biologically active agents may be, without limitation, artificial or naturally occurring small molecules, peptides or polypeptides, immunoglobulins, e.g., antibodies, nucleic acids, etc. Without limitation, hormones, growth factors, drugs, cytokines, chemokines, clotting factors and endogenous clotting inhibitors, etc., are biologically active agents.

The term “endoscope” means a small diameter tube-like instrument, usually employing fiber optics, designed to be inserted through an incision in the body, used for visualization and manipulation during minimally invasive surgical procedures. The term includes “laparoscopes,” which are designed for visualization and manipulation of tissues and organs in the abdominopelvic cavity and “arthroscopes,” which are designed for visualization and manipulation of tissues within the joint space, etc.

“Fibrinolytic agent,” as used herein, refers to any substance that directly or indirectly contributes to the degradation of fibrin.

A “HAX hydrogel” is a hydrogel formed from crosslinked HA derivatives.

A “hybrid hydrogel” is a composite hydrogel comprised of particles and crosslinked polysaccharide derivatives.

A “hydrogel” is a three-dimensional network comprising hydrophilic polymers that contains a large amount of water. A hydrogel may, for example contain 30%, 40%. 50%, 60%, 70%, 80%, 90%, or an even greater amount of water on a w/w basis. A “hydrogel precursor” is a polymer that is at least partly soluble in an aqueous medium and is capable of becoming crosslinked to form a hydrogel.

“Inhibiting adhesions” refers to administering a composition and/or performing a procedure so as to cause a reduction in the number of adhesions, extent of adhesions (e.g., area), and/or severity of adhesions (e.g., thickness or resistance to mechanical or chemical disruption) relative to the number, extent, and/or severity of adhesions that would occur without such administration. The composition or procedure may inhibit formation, or growth of adhesions following an adhesion promoting stimulus, may inhibit progression of adhesions, and/or may inhibit recurrence of adhesions following their spontaneous regression or following mechanical or chemical disruption.

“In situ” means that a hydrogel is formed substantially at a location in which the hydrogel is desired rather than being formed elsewhere and subsequently applied to a location at which it is desired. For example, formation of a hydrogel on or within the body of a subject is considered to be “in situ” for purposes of the present invention.

“Liposomes” are artificial microscopic spherical particles formed by a lipid bilayer (or multilayers) enclosing an aqueous compartment, which may contain a biologically active agent.

“Extended local peritoneal administration” refers to administering one or more compositions so that the compositions collectively contact a portion of the peritoneum at least equal in area to the area that lies within a distance of 10 cm from the borders of a site of damage, e.g., so that a portion of peritoneum whose area is at least equal to the area that lies within a distance of 10 cm from the borders of a site of damage is covered by a hydrogel layer upon crosslinking of the polysaccharide derivatives present in the composition(s). A site that has suffered damage may be any site at which the the peritoneum is visibly physically or functionally compromised, e.g., a surgical incision, an injury, a site at which infection has resulted in a visible physical alteration in the peritoneum (e.g., inflammation, exudate, etc.). The area covered may, for example, be contiguous with and surround the site of damage or may be located on an opposite surface of the peritoneum. For example, the damage may be a surgical incision in the abdominal wall so that the parietal peritoneum is damaged, and the area covered may be on the visceral peritoneum located opposite to the site of damage.

“Pan-peritoneal administration” refers to administering one or more compositions so that the compositions collectively contact a substantial portion of the peritoneum (e.g., at least 10% of the surface area of the peritoneum) following administration, e.g., so that at least 10% of the surface area of the peritoneum is covered by a hydrogel layer upon crosslinking of the polysaccharide derivatives present in the composition(s).

“Particle” refers to a small object, fragment, or piece of material and includes, without limitation, polymeric particles, biodegradable particles, non-biodegradable particles, single-emulsion particles, double-emulsion particles, coacervates, liposomes, microparticles, nanoparticles, macroscopic particles, pellets, crystals, aggregates, composites, pulverized, milled or otherwise disrupted matrices, cross-linked protein or polysaccharide particles (including particles comprising HAX). Particles may be composed of a single substance or multiple substances. In certain embodiments of the invention the particle is not a viral particle.

The term “peritoneum” refers to the serous membrane that lines the walls of the abdominopelvic cavity, which extends from the inferior surface of the diaphragm to the superior surface of the pelvic floor. In addition, the peritoneum at least in part covers various abdominopelvic organs, e.g., bowel, stomach, liver, kidneys, adrenal glands, spleen, bladder, uterus, ovaries, and fallopian tubes. A film of fluid lubricates the surfaces of the peritoneum and normally facilitates free movement of the viscera against another or against the abdominal or pelvic walls. The term “peritoneal adhesions” refers to adhesions that occur in the peritoneal cavity. Peritoneal adhesions attach organs or tissues to one another or to the walls of the abdominopelvic cavity.

“Preventing adhesions” refers to administering or applying a therapeutic composition and/or procedure prior to formation of adhesions in order to reduce the likelihood that adhesions will form in response to a particular insult, stimulus, or condition. It will be appreciated that “preventing adhesions” does not require that the likelihood of adhesion formation is reduced to zero. Instead, “preventing adhesions” refers to a clinically significant reduction in the likelihood of adhesion formation following a particular insult or stimulus, e.g., a clinically significant reduction in the incidence or number of adhesions in response to a particular adhesion promoting insult, condition, or stimulus.

“Small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

“Solubility” refers to the amount of a substance that dissolves in a given volume of solvent at a specified temperature and pH, e.g., to form a saturated solution. Solubility may be determined, for example, using the shake-flask solubility method (ASTM: E 1148-02, Standard Test Method for Measurements of Aqueous Solubility, Book of Standards Volume 11.05). Solubility may be determined at a pH between 3.0 and 9.0, e.g., between 4.0 and 8.0, between 5.0 and 8.0, between 6.0 and 8.0, e.g., between 6.5 and 7.6, e.g., between 6.8-7.4, e.g., 7.0, or any intervening value of the foregoing ranges. Solubility may be tested at a temperature of between 20 and 40° C., e.g., approximately 25-37° C., e.g., approximately 37° C., or any intervening value of the foregoing ranges. For example, solubility may be determined at approximately pH 7.0-7.4 and approximately 37° C.

“Subject,” as used herein, refers to an individual to whom an agent is to be delivered, e.g., for experimental, diagnostic, and/or therapeutic purposes. Preferred subjects are mammals, particularly domesticated mammals (e.g., dogs, cats, etc.), primates, or humans. A subject under the care of a physician or other health care provider may be referred to as a “patient.”

A “sustained release formulation” is a composition of matter that comprises a biologically active agent as one of its components and further comprises one or more additional components, elements, or structures effective to provide sustained release of the therapeutic agent, optionally in part as a consequence of the physical structure of the formulation. Sustained release is release or delivery that occurs either continuously or intermittently over a period of time, e.g., at least 2, 3, 4, 5, or 6 days, at least 1, 2, 4, or 6 weeks, up to about 1, 2, 3, 4, 6, 8, 10, 12, 15, 18, or 24 months.

“Therapeutic agent,” also referred to as a “drug” is used herein to refer to an agent that is administered to a subject to treat a disease, disorder, or other clinically recognized condition that is harmful to the subject, or for prophylactic purposes, and has a clinically significant effect on the body to treat or prevent the disease, disorder, or condition. Therapeutic agents include, without limitation, agents listed in the United States Pharmacopeia (USP), Goodman and Gilman's The Pharmacological Basis of Therapeutics, 10^{th} Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17^{th} ed. (1999), or the 18th ed (2006) following its publication, Mark H. Beers and Robert Berkow (eds.), Merck Publishing Group, or, in the case of animals, The Merck Veterinary Manual, 9^{th} ed., Kahn, C. A. (ed.), Merck Publishing Group, 2005.

“Treating adhesions,” as used herein, refers to administering or applying a composition and/or procedure that reverses, alleviates, reduces, and/or inhibits the progression and/or severity of adhesions, or reduces the likelihood of recurrence and/or the severity of recurrent adhesions following a procedure intended to disrupt or reduce the extent or severity of adhesions. “Treating adhesions” also refers to administering or applying a composition and/or procedure that reverses, alleviates, reduces, inhibits the progression of, or reduces the likelihood of recurrence and/or severity of one or more symptoms of adhesions (e.g., pain, bowel obstruction, infertility). Thus “treating adhesions” involves administering or applying a therapeutic composition and/or procedure once adhesion(s) have already formed following an insult or stimulus.

“Tumor” refers to an abnormal mass of tissue that results from excessive cell division. A tumor can be benign (not cancerous) or malignant (cancerous). “Tumor” includes disorders characterized by excessive division of hematopoietic cells. Such disorders include malignant and premalignant hematologic disorders such as leukemia, lymphoma, myeloma, and myeloproliferative disorders. Tumors can be diagnosed using any of a variety of art-accepted methods including physical diagnosis, imaging studies, histopathology (e.g., performed on a cell or tissue sample), biochemical tests, etc. Specific, non-limiting examples of tumors include sarcomas, prostate cancer, breast cancer, endometrial cancer, hematologic tumors (e.g., leukemia, Hodgkin's and non-Hodgkin's lymphoma, multiple myeloma and other plasma cell disorders, myeloproliferative disorders), brain tumors (e.g., low grade astrocytoma, anaplastic astrocytoma, glioblastoma multiforme, oligodendroglioma, and ependymoma), and gastrointestinal stromal tumors (GIST). Sarcomas include osteosarcoma, Ewing's sarcoma, soft tissue sarcoma, and leiomyosarcoma. Additional examples of malignant tumors include small cell and non-small cell lung cancer, kidney cancer (e.g., renal cell carcinoma), hepatocellular carcinoma, pancreatic cancer, esophageal cancer, colon cancer, rectal cancer, stomach cancer, breast cancer, ovarian cancer, bladder cancer, testicular cancer, thyroid cancer, head and neck cancer, thyroid cancer, etc. “Tumor” as used herein includes metastases from a primary tumor.

“Viscosity” refers to a measurment of the thickness or resistance to flow of a liquid at a given temperature. Viscosity may be determined using a variety of methods and instruments known in the art. For example, a polymer is first weighed and then dissolved in an appropriate solvent. The solution and viscometer are placed in a constant temperature water bath. Thermal equilibrium is obtained within the solution. The liquid is then brought above the upper graduation mark on the viscometer. The time for the solution to flow from the upper to lower graduation marks is recorded. Viscosity of a solution comprising a polymer may be determined in accordance with ASTM Book of Standards, Practice for Dilute Solution Viscosity of Polymers (ASTM D2857), Volume 08.01, June 2005 or relevant ASTM standards for specific polymers. Solubility may be tested at a temperature of between 20 and 40° C., e.g., approximately 25-37° C., e.g., approximately 37° C., or any intervening value of the foregoing ranges. For example, solubility may be determined at approximately pH 7.0-7.4 and approximately 37° C.

### DETAILED DESCRIPTION OF CERTAIN EMBODIMENTS OF THE INVENTION

I. Anti-Adhesion Compositions Comprising In Situ Cross-Linkable Polysaccharide Derivatives

Adhesions are believed to arise as a result of a complex inflammatory process in which tissues that normally remain separated within the body become attached to one another, usually as a result of surgical trauma, injury, or infection. These adhesions, including adhesions from other causes, are a major cause of severe complications such as bowel obstruction and infertility. Other adhesion-related complications include chronic abdominopelvic pain, urethral obstruction, and voiding dysfunction. Inflammatory processes suspected to play a role in adhesion formation include neutrophil accumulation and activation in the traumatized tissues, fibrin deposition and bonding of adjacent tissues, macrophage invasion, fibroblast proliferation into the area, collagen deposition, angiogenesis and the establishment of permanent adhesion tissues. Current therapeutic approaches include the use of steroidal and non-steroidal anti-inflammatory drugs and a variety of barrier-based approaches involving the application of films or membranes in an attempt to maintain tissue separation. However, the efficacy of these approaches remains limited.

The present invention provides compositions and methods for preventing and/or treating adhesions. The invention arose in part from the inventors' discovery that certain polysaccharides, e.g., derivatives of hyaluronic acid (HA) or cellulose, when administered in solution to a site within the body, e.g., a site of tissue injury or damage, become crosslinked to one another in situ (i.e., at or close to their site of administration within the body) to form a hydrogel that inhibits adhesions. In a first aspect, the invention provides a method of inhibiting adhesions comprising the steps of: administering a first polysaccharide derivative to a location within the body of a subject; and administering a second polysaccharide derivative to the location within the body of the subject, wherein the first and second polysaccharide derivatives become crosslinked to form a hydrogel following contact of the polysaccharide derivatives with one another, and wherein the hydrogel inhibits adhesions. The polysaccharide derivatives are dissolved in solution prior to their administration. The solutions may be administered to the subject substantially simultaneously. The solutions may be mixed to form a single solution prior to administration, in which case they are preferably administered before substantial crosslinking occurs. Derivatives of HA, cellulose, and dextran are chiefly exemplified herein, but the invention contemplates use of other polysaccharides and derivatives thereof and also non-polysaccharide polymer hydrogel precursors in the compositions and methods for inhibiting adhesions and in the other compositions and methods described herein.

The hydrogel forms between tissues or structures that may otherwise come into contact with one another and between which adhesions could therefore develop, e.g., during the process of wound healing. The hydrogel thus serves to separate tissues or structures that have been subjected to injury, trauma, exposure to the external environment, or any other type of insult. The invention therefore provides a method of maintaining separation between tissues or structures comprising the steps of: administering a first polysaccharide derivative to a location within the body of a subject and administering a second polysaccharide derivative to the location within the body of the subject, wherein the first and second polysaccharide derivatives become crosslinked to form a hydrogel following contact of the hydrogel precursors with one another, and wherein the hydrogel is located between tissues or structures to be kept separate from one another.

The hydrogel inhibits the adherence of tissues or structures to one another and inhibits the development of scar-like, fibrous bands between the tissues or structures. The solutions may be administered following an adhesion promoting stimulus, i.e., any event that increases the likelihood of adhesion formation, progression, and/or recurrence. Examples of adhesion promoting stimuli include surgery, injury, and infection. The hydrogel degrades within the body and therefore need not be removed.

The invention provides hydrogels formed by crosslinking a first polysaccharide derivative and a second polysaccharide derivative, wherein the first and second polysaccharides are different. For example, the first polysaccharide may be an HA derivative comprising a first functional group and the second polysaccharide may be a cellulose derivative comprising a second functional group. To give but another example, the first polysaccharide is an HA derivative comprising a first functional group, and the second polysaccharide is a dextran derivative comprising a second functional group. The first and second functional groups may be selected from amine, amide, aldehyde, ester, hydroxy, or hydrazide.

The invention further provides hydrogels formed by crosslinking a first polysaccharide derivative and a second polysaccharide derivative, wherein the first and second polysaccharides are the same and wherein the first polysaccharide derivative comprises a first functional group and the second polysaccharide derivative comprises a second functional group, wherein the first and second functional groups are capable of crosslinking to one another. The polysaccharide may be, e.g., HA, cellulose, dextran, or a derivative of either.

A variety of polysaccharide derivatives may be used. In certain embodiments of the invention at least one of the polysaccharide derivatives is a derivative of HA. In certain embodiments of the invention both of the polysaccharide derivatives are derivatives of HA. Thus the invention provides a method of (i) administering a solution comprising a first HA derivative to a location within the body of a subject; and (ii) administering a solution comprising a second HA derivative to the location within the body of the subject, wherein the first and second HA derivatives become crosslinked to form a hydrogel following contact of the solutions with one another, and wherein the hydrogel inhibits the formation of adhesions. In certain embodiments of the invention the polysaccharide is one that is not specifically degraded by an enzyme endogenous to human beings. Without wishing to be bound by any theory, hydrogels formed at least in part from derivatives of such polysaccharides may have a longer half-life in the body than hydrogels formed from HA derivatives.

In certain embodiments of the invention at least one of the polysaccharide derivatives is a derivative of cellulose. For example, in certain embodiments of the invention the first polysaccharide derivative is a derivative of HA and the second polysaccharide derivative is a derivative of cellulose.

In certain embodiments of the invention at least one of the polysaccharide derivatives is a derivative of dextran. For example, in certain embodiments of the invention the first polysaccharide derivative is a derivative of HA and the second polysaccharide derivative is a derivative of dextran.

HA, also referred to as hyaluronan or hyaluronate, is an unbranched polysaccharide containing repeating disaccharide subunits composed of N-acetyl-D glucosamine and D-glucuronic acid. (See Laurent, T. C. (ed)., Chemistry, Biology and Medical Applications of Hyaluronan and Its Derivatives, London: Portland Press, 1998). The structure of HA may be represented as shown below.

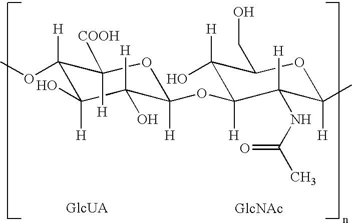

As used herein, the term “hyaluronic acid” (HA) refers to HA and any of its salts, e.g., sodium hyaluronate, potassium hyaluronate, magnesium hyaluronate, calcium hyaluronate, etc. The term “HA derivative” refers to HA that has been chemically modified from the native form represented above. The modifications may include the addition or creation of new functional groups (e.g., amine, amide, aldehyde, ester, hydroxy, hydrazide, etc.), in which case the HA is said to be “functionalized.” The proportion of disaccharide subunits that are modified can vary, and the degree of modification can be selected in order to control properties such as gelation time, half-life, stiffness, etc. Certain modifications retain the native HA backbone structure while other modifications open at least some of the sugar rings. For example, certain modifications open at least some of the sugar rings of the glucuronic acid moieties.

The first and second HA derivatives of the invention comprise first and second functional groups, respectively, that react with one another to form covalent bonds that join the first and second HA derivatives to one another. The solutions are thus applied as liquids and are contacted with one another and optionally mixed together either immediately before or at the time of administration or contact one another following adminstration. Formation of a sufficient number of crosslinks causes a transition from a liquid to a semi-solid or gel-like state.

In certain embodiments of the invention an HA derivative comprising at least two different functional groups is employed, wherein the functional groups react with one another to form crosslinks under physiological conditions. The functional groups may be selected so that they substantially do not react with one another until exposed to physiological conditions of pH, temperature, and/or salt concentration. Thus it will be appreciated that the invention does not require two distinguishable HA derivatives but may instead employ a single species that comprises multiple different functional groups capable of becoming crosslinked.

A variety of different HA derivatives are of use in the invention. An important feature of suitable derivatives is that the first and second functional groups must react in sufficient amounts and with sufficient rapidity so as to allow hydrogel formation within a time frame following contact of the solutions with one another. In certain embodiments of the invention the hydrogel forms within between 1-3 seconds and 5 minutes, between 1-3 seconds and 3 minutes, between 1-3 seconds and 60 seconds, between 1-3 seconds and 30 seconds, or between 1-3 seconds and 15 seconds, following contact of the solutions with one another, e.g., following administration. Typically the solutions are mixed together either immediately before or concurrently with their administration to a site within the body. For example, the solutions may be administered using a multiple barrel injection device, e.g., a multiple barrel syringe, wherein each solution is contained in a separate receptacle or barrel prior to administration. The solutions may contact each other during the administration process and/or thereafter. Preferably the derivatives become crosslinked under physiological conditions, e.g., in an aqueous environment at a pH between 6.0 and 8.0.

A second important feature of suitable HA derivatives is that the resulting hydrogel should not itself contribute significantly to adhesion development, inflammation, or other undesirable effects. Various HA derivatives have been proposed for use as tissue adhesives or glues. In contrast to the HA derivatives of the present invention, such derivatives may exacerbate the problem of adhesions rather than contribute to its solution. Various HA derivatives that offer a suitable environment for cell growth and infiltration have been proposed as scaffolds for tissue regeneration. However, for purposes of inhibiting adhesions, an environment that enhances cellular infiltration and/or proliferation may be undesirable. The present invention identifies polysaccharide derivatives, e.g., HA derivatives, that are suitable for rapid in situ crosslinking and formation of a hydrogel that inhibits adhesions.

A variety of crosslinkable polysaccharide derivatives and methods for forming them may be employed. In certain embodiments of the invention the polysaccharide derivatives become crosslinked to one another without needing a separate crosslinking agent, e.g., the first and second derivatives comprise functional groups that react with one another to form a covalent bond. In certain embodiments of the invention the polysaccharide derivatives react with one another to produce a nontoxic, biocompatible product, e.g., water. In certain embodiments of the invention neither of the polysaccharide derivatives is modified by using a crosslinking agent. In certain embodiments of the invention the polysaccharide derivatives become crosslinked without requiring light.

A wide variety of HA derivatives can be employed in one or more aspects of the instant invention. In certain embodiments of the invention functional groups are introduced into HA by forming an active ester at the carboxyl group (COOH) of the glucuronic acid moiety and performing subsequent substitution with a side chain containing a nucleophilic group on one end and a protected functional group on the other end, e.g., as described in U.S. Pat. No. 6,630,457, which is incorporated herein by reference, and in Bulpitt, P. and Aeschlimann, D., (1999) J. Biomed. Mater. Res., 47, 152-169, which is incorporated herein by reference. This approach can be used, for example, to generate HA derivatives comprising amine or aldehyde functional groups. Active esters of HA can be formed using 1-hydroxybenzotriazole (HOBT) or N-hydroxysulfosuccinimide and then employing a carbodiimide, such as EDC, for coupling. Amines capable of reacting with the ester intermediate formed with HOBT include hydrazines and activated amines such as ethyelene diamine having a pKa in a suitable range such that they are unprotonated at acidic pH (e.g., about 5.5 to 7.0). Use of N-hydroxysulfosuccinimide allows the coupling to be carried out at a pH of about 7.0 to 8.5, allowing the use of primary amines. These approaches can be used to perform the following reactions:
HA-COOH+H₂N—R→HA-CO—NH—R (I)
HA-COOH+R′—NH—R→HA-CO—NR′—R (II)

R and R′ can be any of a wide variety of moieties such as hydrogen, alkyl, aryl, alkylaryl, or arylalkyl, which may contain heteroatoms such as oxygen, nitrogen, and sulfur. The side chain may be branched or unbranched and may be saturated or may contain one or more multiple bonds. The carbon atoms of the side chain may be continuous or may be separated by one or more functional groups such as an oxygen atom, a keto group, an amino group, an oxycarbonyl group, etc. The side chain may be substituted with aryl moieties or halogen atoms, or may in whole or in part be formed by ring structures such as cyclopentyl, cyclohexyl, cycloheptyl, etc. The side chain may have a terminal functional group for crosslinking such as aldehyde, amine, arylazide, hydrazide, maleimide, sulfydryl, ester, carboxylate, imidoester, hydroxyl, etc. Thus HA derivatives comprising any of a large number of different functional groups can be produced using this method.

Carbodiimides suitable for use to form an HA derivative can be represented as follows:
R—N══C══N—R′. (III)

R and R′ can be any of a wide variety of moieties, e.g., as described above. For example R and R′ can be independently selected from the group consisting of: hydrogen, hydrocarbyl of 1-25 carbon atoms and including substituted hydrocarbyl, alkoxy, aryloxy, alkaryloxy and the like. For example, R and R′ can be alkyl, cycloalkyl, aryl or substituted forms thereof. In certain embodiments of the invention a carbodiimide that is at least partially soluble in an aqueous medium, e.g., at temperatures ranging from about 20-80° C. is used. Exemplary carbodiimides include EDC (1-ethyl-3-(3-dimethylaminopropyl)carbodiimide; ETC (1-(-3-dimethylaminopropyl)-3-ethylcarbodiimide methiodide). N,N′-dicyclohexylcarbodiimide; N-allyl-N′-(β-hydroxyethyl)carbodiimide; N-(α-dimethylaminopropyl)-N′tert-butylcarbodiimide; N-(α-dimethylaminopropyl)-N′-(β-bromoallyl)carbodiimide; 1-(3-dimethylaminopropyl)-3-(6 benzoylaminohexyl)carbodiimide, and N-cyclohexyl-N′-beta-(4-methylmorpholinium)ethyl carbodiimide (CMC), etc.

In certain embodiments of the invention the side chain comprises a dihydrazide. The HA derivative comprising a dihydrazide may be formed as described above. Other methods for forming an HA derivative comprising a dihydrazide functional group may also be used. For example, U.S. Pat. No. 5,616,568, which is incorporated herein by reference, teaches a method for functionalizing HA with a dihydrazide to form dihydrazido-HA. A dihydrazide is first added to HA, followed by addition of a carbodiimide. The reaction may be carried out at a pH of about 4-0 and may be represented as follows:
HA—COOH+H₂N—NH—CO-A-CO—NH—NH₂ (dihydrazide) (IV)
θR′—N══C══N—R″ (carbodiimide)
HA—CO—NH—NH—CO-A-CO—NH—NH₂ (dihydrazido-HA) (V)

A wide variety of dihydrazides may be employed in the above reaction, wherein A represents a variable spacer. For example, A may be hydrocarbyl, heterocarbyl, substituted hydrocarbyl, substituted heterocarbyl and the like, wherein these terms are used as described and exemplified in U.S. Pat. No. 5,616,568. In certain embodiments of the invention the dihydrazide has the following formula:
NH₂NHCO(CH₂)ₙ,CONHNH₂ (VI)
wherein n=1 to 18

For example, useful dihydrazides include succinic (butanedioic) (n=2), adipic (hexanedioic) n=4, suberic (octanedioic) (n=6), oxalic (ethanedioic) (n=0), malonic (propanedioic) (n=1), glutaric (pentanedioic) (n=3), pimelic (heptanedioic) (n=5), azelaic (nonanedioic) (n=7), sebacic (decanedioic) (n=8), dodecandioic, (n=10), brassylic (tridecanedioic), (n=11), etc. up to n=20. Suitable carboiimides were discussed above.

The structure of a suitable HA derivative functionalized with a dihydrazide (adipic dihydrazide) is shown below and will be referred to herein as HA-ADH. The arrow indicates the position at which HA is modified (i.e., at the carboxyl group of glucuronic acid moieties).

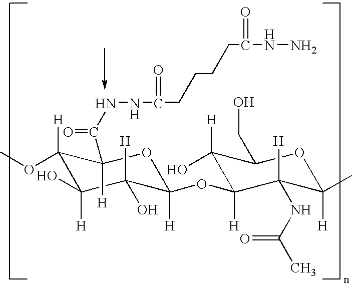

In certain embodiments of the invention, rather than modifying the carboxyl group of glucuronic acid moieties, an HA derivative comprising aldehyde functional groups is formed by oxidizing hydroxyl groups of glucuronic acid moieties to form aldehyde groups. A variety of oxidizing agents may be used, e.g., salts of periodic acid, e.g., potassium periodate (KIO₄), sodium periodate (NaIO₄), or HIO₄. Other oxidizing agents include permanganate, chromate, or dichromate salts.

The structure of this oxidized HA derivative is shown below and will be referred to herein as HA-CHO. The arrow indicates the site of modification.

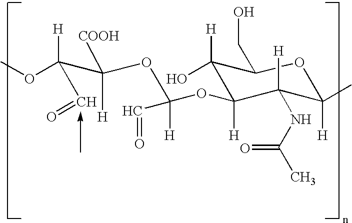

It will be appreciated that in any of the above modification schemes, only a fraction of the sugar moieties in HA become modified. The extent of modification can vary. For example, in certain embodiments of the invention between 5% and 99-100% of the relevant sugar moieties (e.g., glucuronic acid moieties in the case of the modifications described above) are modified. In certain embodiments of the invention between 10% and 75% of the relevant sugar moieties are modified. The extent of modification can be controlled by a variety of methods. For example, the temperature, pH, and time during which the reaction is allowed to proceed can be varied, as can the concentration of the reagents (e.g., carbodiimide, amide, dihydrazide, etc.). To achieve a high degree of modification an excess of the modifying agent(s), e.g., dihydrazide and/or carboiimide, may be used. For example, in one embodiment a 10-100 fold excess of dihydradize is added to a solution comprising HA, and/or a 2-100 fold excess of carbodiimide reagent is then added to the reaction mixture. In certain embodiments of the invention values for these parameters are selected so as to achieve a relatively high degree of modification, e.g., between 50% and 99-100% of the relevant sugar moieties are modified. For example, between 50% and 80% of the relevant sugar moieties may be modified. However, the degree of modification is kept low enough so that the solution will remain in a suitably fluid state rather than becoming too viscous for easy manipulation and syringibility. In certain embodiments of the invention between 10% and 30%, or between 30% and 50% of the relevant sugar moieties are modified.

HA derivatives functionalized as described above can be crosslinked by allowing derivatives comprising different functional groups to react with one another. For example, (i) a first HA derivative comprising an aldehyde can react with a second HA derivative comprising an amine; (ii) a first HA derivative comprising an active ester such as an NHS ester can react with a second HA derivative comprising an amine; (iii) a first HA derivative comprising a maleimide can react with a second HA derivative comprising a sulfhydryl group; (iv) a first HA derivative comprising a hydrazide can react with a second HA derivative comprising an aldehyde, etc. In certain embodiments of the invention the HA derivatives are attached to one another by a bond other than a disulfide bond. In one embodiment of particular interest the first solution contains an HA derivative comprising glucuronic acid moieties that are functionalized with a dihydrazide, and the second solution contains an HA derivative that is oxidized at hydroxyl groups of glucuronic acid moieties of the HA to form aldehyde groups. The first and second HA derivatives become crosslinked forming a hydrazone compound, as shown schematically below, where R₁ and R₂ represent portions of HA (or another polysaccharide such as a cellulose derivative in certain embodiments of the invention).

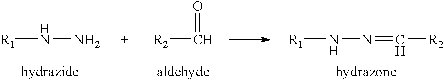

The inventors have shown that hydrogels formed in situ by crosslinking of certain HA derivatives display a remarkable ability to inhibit adhesions (see Examples), even under conditions in which 100% of subjects would develop adhesions in the absence of the hydrogel. Surprisingly, certain hydrogels comprising HA derivatives in which the native HA backbone structure is altered by oxidation of the glucuronic acid ring inhibit adhesions and display low cytotoxicity and good biocompatibility, even though it might be expected that destroying the native HA backbone structure might render the compositions pro-inflammatory and/or immunogenic in vivo.

One aspect of the present invention is the discovery that certain important parameters such as gelation time and degradation rate of hydrogels formed by crosslinking of polysaccharide derivatives such as HA derivatives can be controlled, e.g., by altering the concentration and/or molecular weight of the polysaccharide derivatives in solution. One aspect of the invention involves the discovery that high concentrations of HA derivatives in solution can be achieved by appropriate selection of the molecular weight of the unmodified HA and/or the degree of modification. In particular, it has been discovered that by reducing the molecular weight of the unmodified HA derivatives, the achievable concentration of an HA derivative generated by modifying the HA can be increased. Thus the solubility of HA derivatives generated by modifying the lower molecular weight HA is greater than the solubility of derivatives generated by making the same modification to a higher molecular weight HA, thereby allowing higher concentrations to be achieved without rendering the composition too viscous for easy manipulation and syringibility. The invention provides a composition comprising an HA derivative in solution, wherein the concentration of the HA derivative is greater than 5 mg/ml, e.g., up to 150 mg/ml. In certain embodiments, the concentration of the HA derivative is greater than 10 mg/ml. In other embodiments, the concentration of the HA derivative is greater than 15 mg/ml or greater than 25 mg/ml. The concentration may be at least 26 mg/ml, at least 30 mg/ml, at least 40 mg/ml, at least 50 mg/ml, etc. For purposes of the present invention a concentration of an HA derivative greater than 25 mg/ml is referred to as a “high concentration.” The invention further provides a composition comprising an HA derivative in solution, wherein the concentration of the HA derivative is between 50 mg/ml and 100 mg/ml, e.g., between 50 mg/ml and 75 mg/ml. The solution preferably has a sufficiently low viscosity such that it can be readily manipulated, e.g., so that easy syringibility exists. The HA derivative can be any of the HA derivatives described above. For example, the HA derivative can be HA-ADH or HA-CHO. In certain embodiments of the invention the HA derivative is dissolved in an aqueous medium. The invention further provides methods for making a hydrogel comprising contacting a first solution comprising a first HA derivative and a second solution comprising a second HA derivative with one another, wherein at least one of the solutions has a high concentration of an HA derivative. The hydrogels can be used for any of the purposes described herein and optionally comprise a biologically active agent and/or particles.

The invention further provides hydrogels formed by contacting a first solution comprising a first HA derivative and a second solution comprising a second HA derivative, and allowing the derivatives to become crosslinked (optionally in situ), wherein the concentration of the first HA derivative in the first solution, the concentration of the second HA derivative in the second solution, or both, is greater than 5 mg/ml, greater than 10 mg/ml, greater than 15 mg/ml, or greater than 25 mg/ml. In certain embodiments of the invention the concentration of the first HA derivative in the first solution, the concentration of the second HA derivative in the second solution, or both, is between 50 mg/ml and 100 mg/ml, e.g., between 50 mg/ml and 75 mg/ml. As described in the Examples, it has surprisingly been discovered that hydrogels formed from solutions comprising at least one HA derivative at a high concentration display (i) decreased “gelation times” (meaning the time required for HA derivatives to become crosslinked to form a gel following contact with one another); (ii) reduced rates of degradation and thus increased half-life (meaning the time required for the hydrogel wet mass to decrease by 50%); or (iii) both decreased gelation time and reduced degradation rate. For example, the half-life of a hydrogel formed by crosslinking first and second HA derivatives (HA-ADH and HA-CHO) increased from 5 days to 11 days when the concentration of HA-ADH and HA-CHO solutions were increased from 20 mg/ml to 75 mg/ml and 30 mg/ml, respectively, and to 22.5 or 51 days when the concentrations were increased to 75 mg/ml and 60 mg/ml (Examples). The invention therefore provides hydrogels formed by crosslinking HA derivatives, wherein the hydrogels have a half-life greater than 10 days, e.g., between approximately 10 and approximately 50 days.

It will be appreciated that a variety of different methods can be used to measure gelation time and degradation rate. Suitable methods are provided herein. However, it should be understood that while the specific methods employed herein are useful for quantifying the effects of altering the concentration and/or molecular weight, the general findings with respect to control over gelation time and degradation rate are independent of the particular methods used to evaluate these parameters. It should also be understood that while particular HA derivatives have been used to illustrate these aspects of the invention, the invention is in no way limited to those particular derivatives. The invention further provides compositions comprising derivatives of other polysaccharides such as cellulose and dextran, wherein the concentration of the polysaccharide derivative is as described above for HA derivatives. See Examples 12 and 13 below. Such derivatives may include, but are not limited to, functionalized cellulose or functionalized cellulose derivatives. Other derivatives which may be used include, but are not limited to, functionalized dextran and functionalized dextran derivatives. Other natural and synthetic polysaccharides and derivatives thereof may also be used prepare the inventive compositions. Thus the invention includes similar compositions and methods as those described herein for HA derivatives, as applied to other hydrogel precursors, e.g., other polysaccharide derivatives including those mentioned herein, e.g., cellulose derivatives and dextran derivatives.

The remarkable ability of hydrogels formed by crosslinking polysaccharide derivatives in situ to inhibit adhesions is not limited to derivatives of HA. The inventors have shown that hydrogels formed in situ by crosslinking of certain HA derivatives and certain cellulose derivatives have a similar adhesion inhibitory effect (Examples 11 and 12). For example, hydrogels formed by crosslinking an HA derivative and any of a variety of cellulose derivatives in which the native cellulose backbone structure is altered by oxidation of the sugar ring also inhibit adhesions and display good biocompatibility in the peritoneum (Examples 11 and 12).

The invention further provides a method of inhibiting adhesions comprising (i) administering an HA derivative to a location within the body of a subject; and (ii) administering a cellulose derivative to the location within the body of the subject, wherein the HA derivative and the cellulose derivative become crosslinked to form a hydrogel following contact with one another, and wherein the hydrogel inhibits adhesions. The HA derivative and the cellulose derivative may be dissolved in separate solutions that are administered substantially simultaneously to the subject.

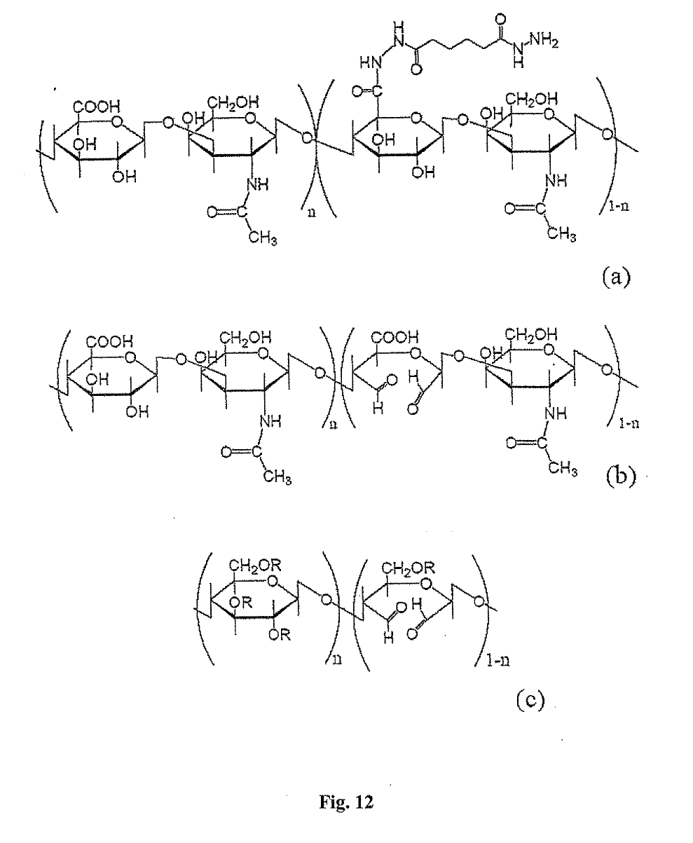

Cellulose is a linear polymer of β-D-glucopyranose units joined to one another (Kamide, Cellulose And Cellulose Derivatives: Molecular Characterization and Its Applications, Elsevier, 2005). The term “cellulose derivative” refers to cellulose that has been chemically modified from this native form. In certain embodiments of the invention the polysaccharide is a cellulose derivative such as methylcellulose (MC), carboxymethylcellulose (CMC), hydroxymethylcellulose (HMC), hydroxypropylcellulose (HPC), hydroxyethyl cellulose (HEC), or hydroxypropyl methylcellulose (HPMC), in which one or more of the OH groups is replaced by OR, wherein R represents any of a variety of moieties. It will be appreciated that some but typically not all of the glucose moieties in any of the afore-mentioned cellulose derivatives are modified (see FIG. 12). In general, crosslinkable cellulose derivatives may be made in a similar manner to that described above for HA derivatives. For example, either cellulose or a cellulose derivative is modified to form a functionalized cellulose derivative that includes a functional group such as an amine, amide, aldehyde, ester, hydroxy, or hydrazide, which is capable of becoming covalently attached to a suitable second functional group, e.g., a functional group on an HA derivative. For example, a cellulose derivative comprising aldehyde functional groups is formed by oxidizing hydroxyl groups of some of the glucose moieties to form aldehyde groups as described herein for HA. Derivatives of MC, CMC, and HPMC formed by oxidizing hydroxyl groups of glucose moieties to form aldehyde groups are referred to herein as MC—CHO, CMC—CHO, and HPMC—CHO respectively (see FIG. 12). Similar nomenclature may be employed for other cellulose derivatives.

The invention provides a composition comprising a cellulose derivative in solution, wherein the concentration of the cellulose derivative is greater than 5 mg/ml, e.g., up to 150 mg/ml. In certain embodiments, the concentration of the cellulose derivative is greater than 10 mg/ml. In other embodiments, the concentration of the cellulose derivative is greater than 15 mg/ml, greater than 20 mg/ml, or greater than 25 mg/ml. For purposes of the present invention a concentration of a cellulose derivative greater than 25 mg/ml is referred to as a “high concentration.” The solution preferably has a sufficiently low viscosity such that it can be readily manipulated, e.g., so that easy syringibility exists. The cellulose derivative can be any of the cellulose derivatives described herein. For example, the cellulose derivative can be MC—CHO, CMC—CHO, and HPMC—CHO.

In certain embodiments of the invention an HA derivative comprising a dihydrazide functional group and a cellulose derivative comprising an aldehyde group are used to form a hydrogel. For example, the first solution may comprise HA-ADH and the second solution may comprise MC—CHO, CMC—CHO, or HPMC—CHO. The HA and cellulose derivatives become crosslinked to form the following hydrazone compounds: HA-CMC, HA-HPMC, and HA-MC.

Furthermore, the inventors have also shown that hydrogels formed in situ by crosslinking of certain HA derivatives and certain dextran derivatives have a similar adhesion inhibitory effect (Example 13). For example, hydrogels formed by crosslinking an HA, cellulose, or other dextran derivative and a dextran derivative in which the native dextran backbone structure is altered also inhibit adhesions and display good biocompatibility in the peritoneum (Example 13).

The invention further provides a method of inhibiting adhesions comprising (i) administering an HA or cellulose derivative to a location within the body of a subject; and (ii) administering a dextran derivative to the location within the body of the subject, wherein the HA or cellulose derivative and the dextran derivative become crosslinked to form a hydrogel following contact with one another, and wherein the hydrogel inhibits adhesions. The HA or cellulose derivative and the dextran derivative may be dissolved in separate solutions that are administered substantially simultaneously to the subject.

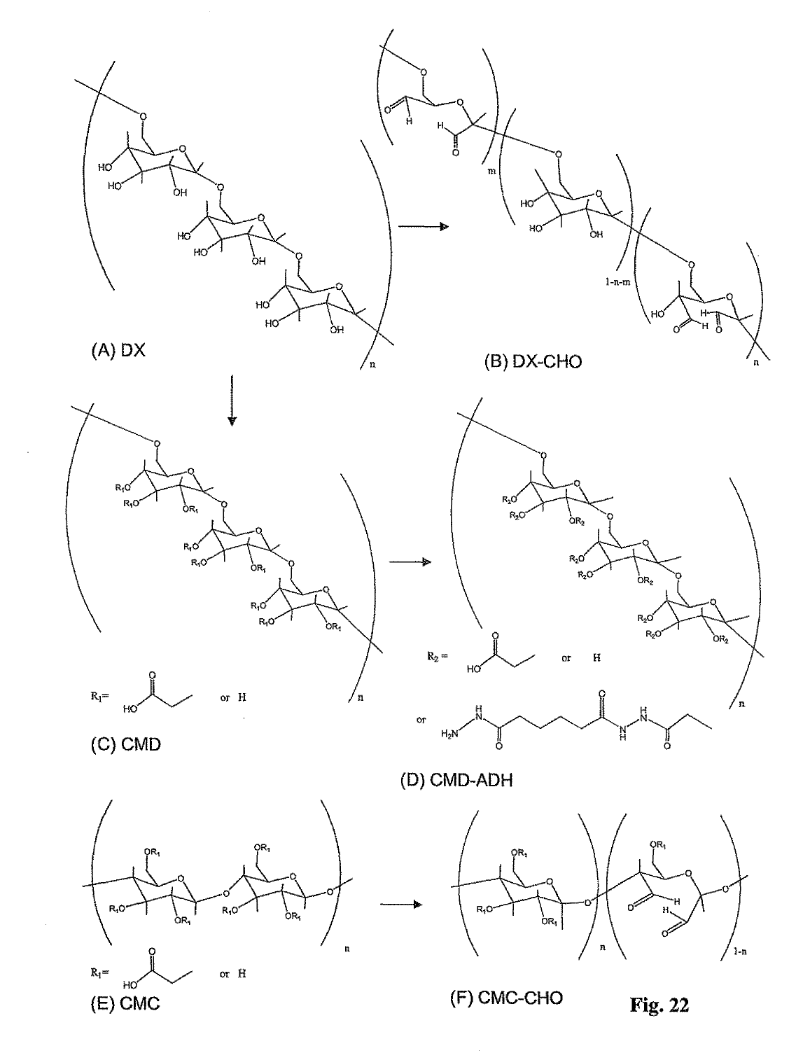

Dextran is a complex, branched polysaccharide. Dextran includes many glucose moieties joined together via α1→6 glycosidic linkages to form straight chains. Branches typically begin from α1→3 linkages, but they may also begins from α1→2 or α1→4 linkages. The structure of a straight chain portion of dextran is shown in FIG. 22. The term “dextran derivative” refers to dextran that has been chemically modified from this native form. In certain embodiments of the invention the polysaccharide is a dextran derivative, in which one or more of the OH groups is replaced by OR, wherein R represents any of a variety of moieties. In other embodiments, the dextran derivative is an aldehyde-containing derivative in which dextran has been treated with periodate. It will be appreciated that some but typically not all of the glucose moieties in any of the afore-mentioned dextran derivatives are modified (see FIG. 22). In general, crosslinkable dextran derivatives may be made in a similar manner to that described above for HA or cellulose derivatives. For example, either dextran or a dextran derivative is modified to form a functionalized cellulose derivative that includes a functional group such as an amine, amide, aldehyde, ester, hydroxy, or hydrazide, which is capable of becoming covalently attached to a suitable second functional group, e.g., a functional group on an HA, cellulose, or dextran derivative. For example, a dextran derivative comprising aldehyde functional groups is formed by oxidizing hydroxyl groups of some of the glucose moieties to form aldehyde groups as described herein for HA and cellulose. Derivatives of dextran formed by oxidizing hydroxyl groups of glucose moieties to form aldehyde groups are referred to herein as DX—CHO (see FIG. 22). Derivatives of carboxymethyldextran (CMDX) modified with hydrazide groups are referred to as CMDX-ADH (see FIG. 22).

The invention provides a composition comprising a dextran derivative in solution, wherein the concentration of the dextran derivative is greater than 5 mg/ml, e.g., up to 150 mg/ml. In certain embodiments, the concentration of the dextran derivative is greater than 10 mg/ml. In other embodiments, the concentration of the dextran derivative is greater than 15 mg/ml, greate than 20 mg/ml, or greater than 25 mg/ml. For purposes of the present invention a concentration of a dextran derivative greater than 25 mg/ml is referred to as a “high concentration.” The solution preferably has a sufficiently low viscosity such that it can be readily manipulated, e.g., so that easy syringibility exists. The dextran derivative can be any of the dextran derivatives described herein. For example, the dextran derivative can be DX—CHO, or CMDX-ADH.

In certain embodiments of the invention an HA or cellulose derivative comprising a dihydrazide functional group and a dextran derivative comprising an aldehyde group are used. For example, the first solution may comprise HA-ADH and the second solution may comprise DX—CHO. The HA and dextran derivatives become crosslinked to form HA-DX. In certain embodiments of the invention a cellulose derivative comprising a dihydrazide functional group and a dextran derivative comprising an aldehyde group are used. For example, the first solution may comprise CMC-ADH and the second solution may comprise DX—CHO. To give but another example, the first solution may comprise CMDX-ADH and the second solution may comprises CMC—CHO. The cellulose and dextran derivatives become crosslinked to form CMC-DX. In certain embodiments of the invention, a dextran derivative comprising a dihydrazide functional group and another dextran derivative comprising an aldehyde group are used. For example, the first solution may comprise CMDX-ADH and the second solution may comprise DX—CHO. The cellulose and dextran derivatives become crosslinked to form CMDX-DX.

It will be appreciated that in any of the above embodiments, only a fraction of the available functional groups on the first and second polysaccharide derivatives will become crosslinked. The crosslinking density can be controlled, e.g., by appropriately selecting the molecular weights of the polysaccharide derivatives. Exemplary crosslinking densities range from about 1×10⁶ to about 1×10⁸ mol/cm³. In certain embodiments, the crosslinking density ranges from 3-50×10⁷ mol/cm³.

In certain embodiments of the invention the polysaccharide derivatives are formed into hydrogel microparticles prior to their administration rather than being administered in solution. The hydrogel particles may be suspended in a liquid medium and administered to a location in the body.

In certain embodiments of the invention at least one of the polysaccharide derivatives suitable for in situ crosslinking to form a composition that inhibits adhesions, and/or for any of the other purposes described herein, comprises a portion that comprises a non-polysaccharide polymer, e.g., the polysaccharide derivative comprises a polysaccharide or derivative thereof covalently attached to one or more non-polysaccharide polymers. Non-polysaccharide means that the polymer contains less than 1% sugar monomers by weight, number, or both, e.g., the polymer contains essentially no sugars. In certain embodiments of the invention the non-polysaccharide portion comprises between 1% and 10%-90% of the polymer by weight and/or between 1% and 10%-90% of the monomers are non-sugar monomers. The attachment may occur at any position in the polysaccharide chain, e.g., either of the ends of the chain or at one of the internally located sugar moieties resulting in either a linear or branched structure. The non-polysaccharide polymer can be any of a variety of polymers, e.g., any non-polysaccharide polymer capable of serving as a hydrogel precursor when covalently attached to a polysaccharide derivative. Typically the polymer is a hydrophilic polymer, i.e., it has an affinity for, and is soluble in, water. Suitable non-polysaccharide polymers include, but are not limited to, polyethers such as polyethyelene glycol (PEG) or polypropylene glycol (PPG), polyethyelene oxides (PEO), polyvinyl alcohol (PVA), polyvinyl pyrrolidone (PVP), polypeptides such as gelatin, poly(l-glutamic acid), polylysine (PLL) and derivatives of any of these, or conjugates, blends, or composites comprising any of these. For example, in one embodiment at least one of the polysaccharide derivatives is an HA derivative having PEG or a PEG derivative covalently attached thereto, wherein either the HA portion or the PEG portion comprises a first functional group. The second polysaccharide derivative can be any polysaccharide derivative comprising a functional group that reacts with the first functional group. For example, in one embodiment the HA derivative is PEG-HA-ADH, and the second polysaccharide derivative comprises a functional group that reacts with a dihydrazide, e.g., HA-CHO. A variety of PEG derivatives comprising suitable functional groups to facilitate formation of covalent bonds are commercially available. See, e.g., Nektar Advanced Pegylation 2005-2006 Product Catalog, Nektar Therapeutics, San Carlos, Calif., which describes a number of these compounds.

In certain embodiments of the invention first and second crosslinkable hydrogel precursors are employed, wherein one of the hydrogel precursors comprises or consists of a polysaccharide derivative such as an HA, cellulose, or dextran derivative and the other hydrogel precursor comprises or consists of a non-polysaccharide polymer (i.e., less than 1% of the polymer by weight, or less than 1% of the monomers are sugars). Exemplary non-polysaccharide polymers capable of becoming crosslinked to a polysaccharide derivative to form a hydrogel include but are not limited to polyethers such as polyethyelene glycol (PEG) or polypropylene glycol (PPG), polyethyelene oxides (PEO), polyvinyl alcohol (PVA), polyvinyl pyrrolidone (PVP), polypeptides such as gelatin, chitosan, or poly(1-glutamic acid), and derivatives of any of these, or conjugates, blends, or composites comprising any of these. In an exemplary embodiment the first crosslinkable hydrogel precursor is HA-ADH and the second crosslinkable hydrogel precursor is a PEG derivative comprising an aldehyde group.

While polysaccharide derivatives are described in detail herein to exemplify the invention, in yet other embodiments of the invention the hydrogel is formed by crosslinking two non-polysaccharide polymers in situ, resulting in a hydrogel that inhibits adhesions. Each of the non-polysaccharide polymers comprises a functional group, wherein the functional groups are capable of reacting with one another to form covalent bonds. Suitable functional groups are those described above for crosslinking of polysaccharide derivatives. Exemplary non-polysaccharide polymers include those described above that contain or may be modified to contain suitable functional groups for crosslinking.

II. Compositions Comprising Crosslinkable Polysaccharide Derivatives and a Biologically Active Agent

The invention further provides compositions as described above in which the hydrogel is formed by crosslinking hydrogel precursors so as to produce a hydrogel that comprises a biologically active agent. For example, either the solution containing the first polysaccharide derivative, the solution containing the second polysaccharide derivative, or both, comprises one or more biologically active agent(s). The hydogel formed by crosslinking the first and second polysaccharide derivatives therefore contains one or more biologically active agents. In some embodiments of the invention each of the solutions comprises a different biologically active agent. The hydrogel formed therefrom contains two or more biologically active agents. Alternately, the solutions containing the first and second hydrogel precursors can be combined with one or more additional solutions each containing one or more biologically active agents. In another embodiment a biologically active agent is added immediately to the composition formed by combining the first and second solutions.

The polysaccharide derivatives can be, e.g., HA derivatives, cellulose derivatives, dextran derivatives, etc. In certain embodiments of the invention the first and second polysaccharide derivatives are HA derivatives. In certain embodiments of the invention the first and second polysaccharide derivatives are cellulose derivatives. In certain embodiments of the invention the first and second polysaccharide derivatives are dextran derivatives. In certain embodiments of the invention the first polysaccharide derivative is an HA derivative and the second polysaccharide derivative is a cellulose derivative. In certain embodiments of the invention the first polysaccharide derivative is an HA derivative and the second polysaccharide derivative is a dextran derivative. In certain embodiments of the invention the first polysaccharide derivative is a cellulose derivative and the second polysaccharide derivative is a dextran derivative. In certain embodiments of the invention, the hydrogel is formed from three or more polysaccharide derivatives. Any combination of HA derivatives, cellulose derivatives, dextran derivatives, other polysacharide derivative, or other polymers may be cross-linked to form the inventive hydrogel.

Any biologically active agent can be included in a hydrogel formed by crosslinking polysaccharide derivatives. The invention therefore provides hydrogels containing any of a wide variety of biologically active agents. The invention further provides a solution containing a polysaccharide derivative such as an HA, cellulose, or dextran derivative and any of a wide variety of biologically active agents

The invention further provides a method of preparing a hydrogel comprising a biologically active agent, the method comprising steps of: contacting a first solution comprising a first polysaccharide derivative and a second solution comprising a second polysaccharide derivative with each other, wherein at least one of the solutions comprises a biologically active agent, and wherein the first and second polysaccharide derivatives comprise functional groups that crosslink to one another. The invention further provides a method of preparing a hydrogel comprising a biologically active agent, the method comprising steps of: contacting a first solution comprising a first polysaccharide derivative, a second solution comprising a second polysaccharide derivative, and a biologically active agent with each other, wherein the first and second polysaccharide derivatives comprise functional groups that crosslink to one another. Optionally the biologically active agent is in solution. In certain embodiments of the invention the hydrogel is prepared by administering the solutions to a subject, wherein the hydrogel precursors crosslink to one another to form a hydrogel that encapsulates the biologically active agent.

In certain embodiments of the invention the biologically active agent is a therapeutic agent. Useful classes of biologically active agents include anti-infective agents, anti-inflammatory agents, anti-proliferative agents (e.g., cytotoxic agents), anti-neoplastic agents (i.e., agents that inhibit or prevent the proliferation of malignant cells and/or inhibit or prevent the growth or spread of tumors), analgesic agents (i.e., agents that relieve pain), anti-oxidants, angiogenesis inhibitors, immunosuppressive agents, immunomodulatory agents, anti-coagulants (i.e., agents that inhibit or prevent formation of blood clots but do not dissolve existing clots, also referred to as anti-thrombogenic agents), proteolytic agents or agents that enhance proteolysis (some of which are also anti-thrombogenic agents), free radical scavengers, anti-oxidants, inhibitors of fibrous repair (e.g., anti-TGF-β agents, D-penicillamine, pentoxifyllin), etc. Other useful classes of therapeutic agents include hypnotics, sedatives, tranquilizers, anti-convulsants, muscle relaxants, antispasmodics, sympathiomimetic agents, cardiovascular agents (e.g., anti-arrhythmic agents, inotropic agents), etc. In certain embodiments of the invention the biologically active agent is not an anesthetic. In certain embodiments of the invention the biologically active agent is not an anti-proliferative agent. In certain embodiments, the biologically active agent is an anti-inflammatory agent. In certain embodiments, the biologically active agent is a non-steroidal anti-inflammatory agent. In other embodiments, the biologically active agent is a steroidal anti-flammatory agent (e.g., a glucocorticoid, corticosteroid).

A biologically active substance is preferably added in amounts that will be pharmaceutically effective when an appropriate amount of the solution comprising a polysaccharide derivative and/or non-polysaccharide polymer is administered to a subject, which can vary. The agent may or may not inhibit adhesions. It will be appreciated that some agents fall into more than one class and may have more than one mechanism of action. The listing of a particular agent as a member of a class is not intended to be limiting.

Exemplary classes of anti-infective agents of use in the invention quinolones, β-lactams (e.g., penicillins or cephalosporins), carbapenems, aminoglycosides, macrolides, lincosamides, ketolides, tetracyclines, glycycyclines, lincomycins, oxazolidinones, amphenicols, ansamycins, polymyxins, aminomethlycyclines, lincosamides, streptogramins, 2,4-diaminopyrimidines, nitrofurans, sulfonamides, sulfones, rifabutins, dapsones, peptides, glycopeptides, and nucleoside analogs. In certain embodiments of the invention the anti-infective agent is one with a broad spectrum of activity against a variety of bacterial species. In some embodiments the agent is effective against one or more species of gram positive bacteria, one or more species of gram negative bacteria, or both. In certain embodiments of the invention the agent is effective against bacteria or fungi that are likely to contaminate surgical wounds or injuries. For example, the agent may be effective against bacteria or fungi commomly found on the skin or in the gastrointestinal tract.

Examples of specific anti-infective agents that can be used include, but are not limited to, erythromycin, nafcillin, cefazolin, imipenem, aztreonam, gentamicin, sulfamethoxazole, vancomycin, ciprofloxacin, trimethoprim, rifampin, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linezolid, temafloxacin, tosufloxacin, clinafloxacin, sulbactam, clavulanic acid, amphotericin B, fluconazole, itraconazole, ketoconazole, and nystatin.

Exemplary classes of anti-inflammatory agents of use in the invention include a wide variety of non-steroidal anti-inflammatory agents (e.g., cyclooxygenase-1 inhibitors), corticosteroids,glucocorticoids, and anti-inflammatory antibodies or polypeptides.

Exemplary anti-inflammatory agents include prednisone; dexamethasone, fluorometholone; prednisolone; methylprednisolone; clobetasol; halobetasol; hydrocortisone; triamcinolone; betamethasone; fluocinolone; fluocinonide; loteprednol; medrysone; rimexolone; celecoxib; folic acid; diclofenac; diflunisal; fenoprofen; flurbiprofen; indomethacin; ketoprofen; meclofenamate; meclofamate; piroxicam; sulindac; salsalate; nabumetone; oxaprozin; tolmetin; hydroxychloroquine sulfate; rofecoxib; etanercept; infliximab; leflunomide; naproxen; oxaprozin; piroxicam; salicylates; valdecoxib; sulfasalazine; methylprednisolone; ibuprofen; budesonide, meloxicam; methylprednisolone acetate; gold sodium thiomalate; aspirin; azathioprine; triamcinolone acetonide; propxyphene napsylate/apap; folate; nabumetone; diclofenac; ketorolac; piroxicam; etodolac; diclofenac sodium; diclofenac potassium; oxaprozin; methotrexate; minocycline; tacrolimus (FK-506); sirolimus (rapamycin) and rapamycin analogs; phenylbutazone; diclofenac sodium/misoprostol; acetaminophen; indomethacin; glucosamine sulfate/chondroitin; and cyclosporine or analogs thereof, etc. Exemplary non-steroidal anti-inflammatory agents (NSAIDs) include celecoxib, diclofenac, diflunisal, etodolac, salicylates, fenoprofen, ibuprofen, flurbiprofen, indomethacin, ketoprofen, ketorolac, meclofamate, meclofenamate, meloxicam, naproxen, piroxicam, sulindac, salsalate, nabumetone, aspirin, oxaprozin, and tolmetin. Exemplary anti-inflammatory steroidal agents include dexamethasone, fluorometholone, prednisolone, loteprednol, medrysone, prednisone, methylpredisolone, cortisone, budesonide, rimexolone, clobetasol, halobetasol, hydrocortisone, triamcinolone, betamethasone, fluocinolone, and fluocinonide. Additional anti-inflammatory agents of interest include an antibody that binds to TNF-α (e.g., infliximab, Remicade®) and a polypeptide that is a soluble TNF-α receptor (e.g., etanercept; Enbrel®).

Additional examples of anti-inflammatory agents cytokine suppressive anti-inflammatory drug(s) (CSAIDs); anti-TNFα antibodies (see, e.g., U.S. Pub. No. 20040126372, incorporated herein by reference); cA2/infliximab (chimeric anti-TNFα antibody; Centocor); IL-4 (anti-inflammatory cytokine; IL-10; IL-10 and/or IL-4 agonists (e.g., agonist antibodies or small molecules); IL-1 receptor antagonist; TNF-bp/s-TNF (soluble TNF binding protein; phosphodiesterase Type IV inhibitor; thalidomide and thalidomide-related drugs; leflunomide; tranexamic acid and other inhibitors of plasminogen activation; prostaglandin E1, tenidap, anti-IL-12 antibodies; anti-IL-18 antibodies; interleukin-11; interleukin-13; interleukin-17 inhibitors; gold; penicillamine; chloroquine; hydroxychloroquine; chlorambucil; cyclophosphamide; cyclosporine; total lymphoid irradiation; anti-thymocyte globulin; anti-CD4 antibodies; CD5-toxins; orally-administered peptides and collagen; lobenzarit disodium; ICAM-1 short interfering RNAs (siRNAs) or antisense oligonucleotides; soluble complement receptor 1.

Angiogenesis inhibitors of use in the invention include agents that inhibit or antagonize vascular endothelial growth factor (VEGF) or its receptor(s), referred to herein as “anti-VEGF agents.” Useful agents include, for example, antibodies, antibody fragments, and nucleic acids that bind to one or more VEGF isoforms or VEGF receptors. The binding may, for example, inhibit interaction of one or more VEGF isoforms with its receptor(s). Avastin (Genentech) is a full length humanized antibody that also binds to VEGF (reviewed in Ferrara, N. Endocr Rev., 25(4):581-611, 2004). Lucentis (Genentech) is a humanized antibody fragment that binds and inhibits Vascular Endothelial Growth Factor A (VEGF-A). (Gaudreault, J., et al., Invest Ophthalmol. Vis. Sci. 46, 726-733 (2005) and references therein. Macugen (Pfizer, Eyetech) is a VEGF nucleic acid ligand (also referred to as an aptamer) that binds to and inhibits VEGF₁₆₅ (U.S. Pat. No. 6,051,698). These and other aptamers or antibodies that bind to one or more VEGF isoforms are of use in the invention.

Other angiogenesis inhibitors include various endogenous or synthetic peptides such as angiostatin, arresten, canstatin, combstatin, endostatin, thrombospondin, and tumstatin. Other anti-angiogenic molecules include thalidomide and its anti-angiogenic derivatives such as iMiDs (Bamias A, Dimopoulos M A. Eur J Intern Med. 14(8):459-469, 2003; Bartlett J B, Dredge K, Dalgleish A G. Nat Rev Cancer. 4(4):314-22, 2004).

Exemplary classes of anti-proliferative agents include alkalizing or alkylating agents, alkyl sulfonates, aziridines, ethylenimines and methylamelamines, nitrogen mustards, antibiotics, anti-metabolites, folic acid analogues, purine analogs, pyrimidine analogs, androgens, anti-androgens, anti-adrenals, arabinosides, anti-estrogens, taxoids, platinum analogs, microtubule inhibitors (e.g., microtubule depolymerizing agents or stabilizers), topoisomerase inhibitors, histone deacetylase (HDAC) inhibitors, aggresome inhibitors, proteasome inhibitors, proapoptotic agents, kinase inhibitors, radioisotopes, animal, plant, or bacterial toxins, etc.

Specific examples of agents falling into these classes include alkalyzing or alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaorami-de and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin (Adriamycin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-androgens, e.g., androgen receptor antagonists such as spironolactone, flutamide, finasteride; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethyl-amine; urethan; vindesine; dacarbazine; mannomustine; mitobronitol; mitolactol; pipobroman; gacytosine; arabinoside (“Ara-C”); thiotepa; taxoids, e.g. paclitaxel and doxetaxel; chlorambucil; gemcitabine; 6-thioguanine; mercaptopurine; methotrexate; platinum analogs such as cisplatin and carboplatin; platinum; etoposide (VP-16); ifosfamide; mitomycin C; mitoxantrone; vincristine; vinblastine; vinorelbine; navelbine; novantrone; teniposide; daunomycin; aminopterin; xeloda; ibandronate; CPT-11; topoisomerase inhibitor RFS 2000; diphtheria toxin; ricin; pseudomonas toxin A, conotoxins, difluoromethylomithine (DMFO); retinoic acid; esperamicins; capecitabine; HER-2 antibody (Herceptin®); kinase inhibitors such as Gleevec®, and pharmaceutically acceptable salts, acids or derivatives of any of the above.

Proteolytic agents suitable for use in the present invention include components of the tissue plamsinogen activator (tPA)/plasmin cascade. Components of the tPA/plasmin cascade include plasminogen activators such as tissue plasminogen activator (tPA) and variants thereof, plasminogen, and plasmin. Plasminogen activators (PAs) are serine proteases that catalyze the conversion of plasminogen to plasmin (Vassalli 1991) by cleavage of a single peptide bond (R561-V562) yielding two chains that remain connected by two disulfide bridges. Plasmin is a potent serine protease whose major substrate in vivo is fibrin, the proteinaceous component of blood clots. Plasminogen activation by tPA is stimulated in the presence of fibrin. Plasmin has a broad substrate range and is capable of either directly or indirectly cleaving many other proteins, including most proteins found in the ECM. “Direct,” as used here, means that the protease physically interacts with the polypeptide that is cleaved, while “indirect” means that the protease does not physically interact with the polypeptide that is cleaved—instead it interacts with another molecule, e.g., another protease, which in turn directly or indirectly cleaves the polypeptide. Plasmin is also capable of activating metalloprotease precursors. The metalloproteases in turn degrade ECM molecules. Metalloproteases are also of use in certain embodiments of the present invention.

Two PAs, tissue-type PA (tPA) and urokinase-type PA (uPA) have been identified in mammals. A major physiological function of PAs to trigger the lysis of clots by activating plasminogen to plasmin, which degrades fibrin. tPA for use in the present invention may be from any species, although for administration to humans it is generally preferred to use human tPA or a variant thereof. In an embodiment of particular interest, the biologically active agent is a tPA. As described in Example 10, hydrogels containing tPA displayed increased ability to inhibit adhesions in the context of tenacious adhesions such as those that may form after multiple damaging events. tPA and useful variants thereof, including variants with improved properties are described in U.S. Pat. Nos. 6,284,247; 6,261,837; 5,869,314; 5,770,426; 5,753,486 5,728,566; 5,728,565; 5,714,372; 5,616,486; 5,612,029; 5,587,159; 5,520,913; 5,520,911; 5,411,871; 5,385,732; 5,262,170; 5,185,259; 5,108,901; 4,766,075; 4,853,330, and other patents assigned to Genentech, Inc. For example, and without limitation, the tPA variant may have an alteration in the protease domain, relative to naturally occurring tPA, and/or may have a deletion of one or more amino acids at the N-terminus, relative to naturally occurring tPA. The tPA variant may have one or more additional glycosylation sites relative to naturally occurring tPA and/or may have an alteration that disrupts glycosylation that would normally occur in naturally occurring tPA when expressed in eukaryotic cells, e.g., mammalian cells. Properties that may be of use include, but are not limited to, increased half-life, increased activity, increased affinity or specificity for fibrin, etc.

Human tPA has been assigned Gene ID 5327 in the Entrez Gene database (National Center for Biotechnology Information; NCBI) and the GenBank entry for the full length amino acid, mRNA, and gene sequences are AAA98809, K03021, and NM_{—}000930, respectively. However, it is noted that it may be preferable to use the mature form of tPA, lacking the signal sequence peptide, as described, e.g., in U.S. Pat. No. 4,853,330, or a variant thereof.

The chymotrypsin family serine proteases, of which tPA is a member, are normally secreted as single chain proteins and are activated by a proteolytic cleavage at a specific site in the polypeptide chain to produce a two chain form. Both the single chain and two chain forms are active towards plasminogen, although the activity of the two-chain form is greater. Plasmin activates single-chain tPA to the two-chain form, thus resulting in a positive feedback loop. Either the single chain or the two chain form of tPA, or combinations thereof, may be used in the present invention.

tPA and variants thereof are commercially available and have been approved for administration to humans for a variety of conditions. For example alteplase (Activase®, Genentech, South San Franciso, Calif.) is recombinant human tPA. Reteplase (Retavase®, Rapilysin®; Boehringer Mannheim, Roche Centoror) is a recombinant non-glycosylated form of human tPA in which the molecule has been genetically engineered to contain 355 of the 527 amino acids of the original protein. Tenecteplase (TNKase®, Genentech) is a 527 amino acid glycoprotein derivative of human tPA that differs from naturally occurring human tPA by having three amino acid substitutions. These substitutions decrease plasma clearance, increase fibrin binding (and thereby increase fibrin specificity), and increase resistance to plasminogen activator inhibitor-1 (PAI-1). Anistreplase (Eminase®, SmithKline Beecham) is yet another commercially available human tPA.

Alternate plasminogen activators include streptokinase (Streptase®, Kabikinase®) and urokinase (Abbokinase®), both of which are commercially available.

Other proteolysis-enhancing agents of use in the invention include tPA activators such as Desmodus rotundus salivary plasminogen activator (DSPA) Desmoteplase (Paion, Germany) which is derived from vampire bat saliva (Liberatore G T, et al., Vampire bat salivary plasminogen activator (desmoteplase): a unique fibrinolytic enzyme that does not promote neurodegeneration. Stroke. February 2003;34(2):537-43; which is incorporated herein by reference). Four distinct proteases have been characterized and are referred to as D rotundus salivary plasminogen activators (DSPAs). Full-length vampire bat plasminogen activator (DSPA1) is the variant most intensively studied and exhibits >72% amino acid sequence identity with human tPA. However, two important functional differences are apparent. First, the DSPAs exist as single-chain molecules that are not cleaved into 2 chain forms. Second, the catalytic activity of the DSPAs appears to be dependent on a fibrin cofactor. Urokinase plasminogen activators such as rescupase (Saruplase®, Grunenthal), and microplasmin (a cleavage product of plasminogen) are also of use in various embodiments of the invention. Alfimeprase (Nuvelo) is yet another proteolysis-enhancing agent of use in the present invention. Alfimeprase is a recombinantly produced, truncated form of fibrolase, a known directly fibrinolytic zinc metalloproteinase that was first isolated from the venom of the southern copperhead snake (Agkistrodon contortrix contortrix) (Toombs C F. (2001) Alfimeprase: pharmacology of a novel fibrinolytic metalloproteinase for thrombolysis. Haemostasis. 31(3-6): 141-7, which is incorporated herein by reference). These enzymes breaks down fibrin directly. Fibrolase itself is also of use in the present invention. Also of use are staphylokinase and streptodornase.

In some embodiments of the invention plasmin or mini-plasmin is administered instead of, or in addition to, tPA. A variety of other agents that have plasmin-like activity may also be used. In general, such substances are able to cleave typical plasmin substrates, such as the synthetic substrate S-2251™ (Chromogenix-Instrumentation Laboratory, Milan, Italy), which is a conveniently assayed chromogenic substrate for plasmin and activated plasminogen. Other agents that have tPA-like activity, e.g., they are able to cleave plasminogen and activate it in a similar manner to tPA, can also be used.

Lumbrokinase is a fibrinolytic enzyme or group of enzymes derived from earthworms Lumbricus rubellus. See, e.g., reporting cloning of a gene encoding lumbrokinase (PI239, GenBank Accession No. AF433650) (Ge et al., (2005) Cloning of thrombolytic enzyme (lumbrokinase) from earthworm and its expression in the yeast Pichia pastoris. Protein Expr Purif. July 2005;42(1):20-8, which is incorporated herein by reference). Other fibrinolytic proteases isolated from earthworms are also of use (Cho, I H, et al., (2004) Purification and characterization of six fibrinolytic serine-proteases from earthworm Lumbricus rubellus. J Biochem Mol Biol. Mar. 31, 2004;37(2):199-205, which is incorporated herein by reference). Also of use is nattokinase.

A variety of fibrinolytic enzymes that have been isolated from various worms, insects, and parasites are of use. For example, destabilase, an enzyme present in the leech, hydrolyzes fibrin crosslinks (Zavalova, L., (1996) Genes from the medicinal leech (Hirudo medicinalis) coding for unusual enzymes that specifically cleave endo-epsilon (gamma-Glu)-Lys isopeptide bonds and help to dissolve blood clots. Mol Gen Genet. 253(1-2):20-5; Zavalova L, et al., (2002) Fibrinogen-fibrin system regulators from bloodsuckers. Biochemistry (Mosc). 67(1): 135-42, each of which is incorporated herein by reference).

In some embodiments of the invention plasminogen is administered instead of, or in addition to, tPA.

Additional proteolytic agents or agents that enhance proteolysis include papain, papase, pepsin, trypsin, chymotrypsin, and hyaluronidase.

Other agents having anti-coagulant or anti-thrombogenic activity include heparin, hirudin, ancrod, dicumarol, sincumar, iloprost, L-arginine, dipyramidole and other platelet function inhibitors, polyethers such as polyethylene oxide, etc.

Free radical scavengers or antioxidants include vitamin A, vitamin E, allopurinol, superoxide dismutase, dimethyl sulphoxide, catalase, tremetazidine, ascorbic acid (vitamin C), methylene blue, lazaroids, mangan-desferoxamine.

Analgesic agents include local anesthetics (e.g., sodium channel blockers), centrally acting agents such as narcotic agent (e.g., opiates such as morphine), non-steroidal anti-inflammatory agents, pyrazolone and salicylic acid derivatives, paracetamol (acetaminophen), tramadol, etc. Some other classes of drugs not normally considered analgesics are used to treat neuropathic pain, e.g., tricyclic antidepressants and cetain anticonvulsants, etc.

In certain embodiments of the invention the biologically active agent is one that inhibits gene expression by an RNAi interference mechanism. RNAi is an endogenous cellular sequence-specific gene-silencing mechanism triggered by short nucleic acids containing a double-stranded portion typically about 17-29 nucleotides in length, e.g., about 19-21 nucleotides in length, and optionally one or two single-stranded overhangs, wherein one of the strands of the double-stranded portion corresponds to and is complementary to a target gene (Shankar, P., et al JAMA. (2005) 293(11):1367-73, which is incorporated herein by reference). Agents capable of causing gene silencing by RNAi (referred to herein as RNAi agents) include short interfering RNAs (siRNAs) and molecules such as short hairpin RNAs (shRNAs) that can be processed intracellularly to generate siRNAs. Various RNAi agents can trigger sequence-specific degradation of mRNA or inhibit translation. In one embodiment the RNAi agent is an siRNA comprising two complementary nucleic acid strands, one of which is complementary to a target gene, wherein the strands are about 19-23 nucleotides in length and each strand comprises a 3′ overhang of 1-3 nucleotides in length. It will be appreciated that perfect complementarity between the RNAi agent and the target gene, or between the two portions of the duplex in the RNAi agent is not required, provided that sufficient complementarity exists to allow hybridization to occur. Typically the degree of complementarity will be at least 80%, at least 90%, or more over at least 15 consecutive nucleotides. In certain embodiments the RNAi agent contains a duplex at least 19 nucleotides long having 0, 1, 2, or 3 mismatches, wherein one of the strands hybridizes with a target gene to form a duplex at least 19 nucleotides long having 0, 1, 2, or 3 mismatches. It will be appreciated that although endogenously synthesized RNAi agents are typically composed of RNA, an RNAi agent produced using chemical synthesis can include one or more deoxyribonucleotides or nucleotide analogs, modified backbone structures, etc., in addition to or instead of ribonucleotides linked by phosphodiester bonds.

The invention provides a novel method of delivering an RNAi agent to a subject, the method comprising the step of administering first and second hydrogel precursors and an RNAi agent to a subject, wherein the hydrogel precursors become crosslinked to form a hydrogel following administration to the subject, wherein the hydrogel encapsulates the RNAi agent. The invention further provides compositions containing an RNAi agent. The composition is any of the hydrogels or solutions containing a hydrogel precursor described herein. The hydrogel compositions are of use to locally deliver an RNAi agent to any of a variety of locations in the body, e.g., the abdominopelvic cavity, joint space, the central or peripheral nervous system or a portion thereof (e.g., brain, spinal cord, peripheral nerve). The RNAi agent is released from the hydrogel either by diffusion out of the gel or as the gel degrades.

Any RNAi agent can be administered. In certain embodiments of the invention the RNAi agent is a therapeutic agent of any of the classes discussed above. In an important embodiment the RNAi agent has an adhesion inhibiting effect. For example, the RNAi agent may inhibit expression of gene that encodes a pro-angiogenic, pro-inflammatory, or pro-fibrinogenic polypeptide

The biologically active agent is typically added to one or more of the solutions prior to allowing the solutions to come in contact with one another and/or prior to administration of the solutions. For example, a biologically active agent is added to a solution containing a first HA derivative prior to loading the solution into a device to be used to administer the solution. The biologically active agent and the HA derivative may be dissolved in a liquid medium at the same time, during overlapping time intervals, or sequentially (meaning that one substance is dissolved before the second substance is added). The solution may be mixed or agitated to ensure a uniform distribution of the agent. The total amount of biologically active agent used may vary. For example, the concentration of the agent following its addition to the first or second solutions may range from 1 μg/ml to 1.0 g/ml. In certain embodiments of the invention the concentration is between 10 μg/ml and 100 mg/ml, or between 100 μg/ml and 10 mg/ml. In certain embodiments of the invention the concentration of the agent following its addition to the first or second solutions ranges between 0.1 nM and 10 mM, e.g., between 1 nM and 1 mM, e.g., between 10 nM and 100 nM. The concentration of the biologically active in the hydrogel formed following crosslinking of the first and second polysaccharide derivatives will depend on its concentration in each of the solutions and the relative volumes of the solutions used. Formation of the hydrogel traps the biologically active agents, which may be slowly released from the hydrogel by diffusion and/or as the hydrogel material degrades in the body.

In other embodiments of the invention one or more of the polysaccharide derivatives, e.g., HA derivatives, has a biologically active agent covalently attached thereto. Any of a wide variety of methods can be used to form a covalent bond between a polysaccharide derivative and a biologically active agent. The biologically active agent and the polysaccharide derivative may comprise or be modified to comprise functional groups capable of reacting with one another, e.g., as described above for the reaction of first and second HA derivatives. Alternately, homobifunctional or heterobifunctional crosslinking agents can be used. General methods for conjugation and crosslinking are described in “CrossLinking,” Pierce Chemical Technical Library, available at the Web site having URL www.piercenet.com and originally published in the 1994-95 Pierce Catalog and references cited therein, in Wong S S, Chemistry of Protein Conjugation and Crosslinking, CRC Press Publishers, Boca Raton, 1991; and G. T. Hermanson, Bioconjugate Techniques, Academic Press, Inc., 1995. For example, according to certain embodiments of the invention a bifunctional crosslinking reagent is used to couple a biologically active agent to a polysaccharide derivative such as an HA derivative, a cellulose derivative, or a dextran derivative. In general, bifunctional crosslinking reagents contain two reactive groups, thereby providing a means of covalently linking two target groups. The reactive groups in a chemical crosslinking reagent typically belong to various classes such as succinimidyl esters, maleimides, pyridyldisulfides, and iodoacetamides. In certain embodiments, an agent such as dicyclohexylcarbodiimide (DCC) or DCI is used to active an ester for subsequent conjugation.

Alternately, as noted above, a functional group on the biologically active agent can be directly reacted with a functional group on the polysaccharide derivative. If the biologically active agent does not contain a suitable functional group, such a functional group can be added using any of a variety of methods known in the art. For example, if the biologically active agent is a polypeptide, a lysine residue or terminal amine can be added to provide an amine group. Alternately, the polypeptide can be modified to include a cysteine residue, thereby providing a sulfhydryl group.

Preferably the attachment of a biologically active agent to a polysaccharide derivative does not reduce the biological activity of the agent below useful levels or interfere significantly with crosslinking of first and second polysaccharide derivatives. It may be desirable to employ different functional groups for the attachment of a biologically active agent and for the crosslinking reaction of the two polysaccharide derivatives.

In an embodiment of particular interest dexamethasone or other glucocorticoid is conjugated to an HA, cellulose, or dextran derivative. The polysaccharide derivative may contain a non-polysaccharide polymer portion, e.g., a PEG portion. In another embodiment of particular interest dexamethasone is conjugated to HA or an HA derivative. In another embodiment of particular interest dexamethasone is conjugated to cellulose or a cellulose derivative. In another embodiment of particular interest dexamethasone is conjugated to dextran or a dextran derivative.

In an embodiment of particular interest ibuprofen or other NSAID is conjugated to an HA, cellulose, or dextran derivative. The polysaccharide derivative may contain a non-polysaccharide polymer portion, e.g., a PEG portion. In another embodiment of particular interest an NSAID is conjugated to HA or an HA derivative. In another embodiment of particular interest an NSAID is conjugated to cellulose or a cellulose derivative. In another embodiment of particular interest NSAID is conjugated to dextran or a dextran derivative.

In certain embodiments of the invention the therapeutic agent is covalently linked to a polysaccharide derivative via a bond that is hydrolytically and/or enzymatically labile under physiological conditions. Labile linkages include ester, amide, amidoester, thioester, acid anhydride, carbamide, carbonate, semicarbazone, hydrazone, oxime, iminocarbonate, phosphoester, phophazene, urethane, and anhydride bonds. Other linkages that are readily cleaved in vivo include polypeptides that contain sites that are recognized and cleaved by an endogenous or exogenously provided protease. Exemplary proteases include serine proteases, aspartyl proteases, acid proteases, alkaline proteases, metalloproteases (e.g. matrix metalloproteases), carboxypeptidase, aminopeptidase, cysteine proteases, etc. Cleavage sites for these proteases are known in the art.

The invention further provides compositions (hydrogels and solutions comprising a biologically active agent) in which at least one of the hydrogel precursors is a polysaccharide derivative that comprises a non-polysaccharide polymer portion. The polysaccharide derivative comprising a non-polysaccharide polymer portion can be any of those described in section I. The polysaccharide derivative typically comprises a polysaccharide or derivative thereof covalently attached to one or more non-polysaccharide polymers.

In yet other embodiments the invention provides a hydrogel comprising one or more biologically active agents, wherein the hydrogel is formed by crosslinking two non-polysaccharide polymers. The non-polysaccharide polymers are typically dissolved in solution as described above for polysaccharide derivatives, wherein one or both of the solutions contains a biologically active agent. The solutions contacted with each other, e.g., by administering them to a subject. Each of the non-polysaccharide polymers comprises a functional group, wherein the functional groups are capable of reacting with one another to form covalent bonds. Suitable functional groups are those described above for crosslinking of polysaccharide derivatives. Exemplary non-polysaccharide polymers include those described in Section I that contain or may be modified to contain suitable functional groups for crosslinking.

III. Hybrid Compositions Comprising a Polysaccharide Derivative and Particles

The invention further provides a composition comprising a polysaccharide derivative and a plurality of particles. For example, the invention provides a composition comprising a first HA derivative; and a plurality of particles. The polysaccharide derivative comprises a functional group capable of forming a covalent bond with a second functional group. In certain embodiments of the invention the composition comprises a liquid medium, e.g., an aqueous medium, a first polysaccharide derivative, and a plurality of particles. The particles may be suspended or dispersed in the medium. In certain embodiments of the invention the composition is a hydrogel made by crosslinking first and second polysaccharide derivatives, either or both of which may be an HA derivative. For example, a first solution comprising a HA derivative and further comprising a plurality of particles is contacted with a second solution comprising a second polysaccharide derivative. In certain embodiments of the invention the second polysaccharide derivative is a second HA derivative. The first and second HA derivatives react with one another via functional groups to form covalent crosslinks therebetween, thus forming a hydrogel that entraps the particles therein. In certain embodiments of the invention the second polysaccharide derivative is a cellulose derivative. In certain embodiments of the invention the second polysaccharide derivative is a dextran derivative. The HA derivative and the cellulose or dextran derivative react with one another via functional groups to form covalent crosslinks therebetween, thus forming a hydrogel that entraps the particles therein. It should be noted that the invention is in no way limited by the method in which the hydrogel entrapping the particles is formed.

In certain embodiments, the invention provides a composition comprising a hydrogel precursor and a plurality of particles wherein the hydrogel precursor is capable of forming a hydrogel within between 1 second and 5 minutes following contact with a second hydrogel precursor, e.g., under physiological conditions. In an embodiment of particular interest the hydrogel forms between 1 second and 5 minutes, e.g., between 1 second and 1 minute, between 1 second and 30 seconds, or between 1 second and 15 seconds, after administration to a subject. In certain embodiments of the invention the hydrogel precursor comprises a non-polysaccharide polymer portion or is a non-polysaccharide polymer.

Any of a wide variety of particles can be incorporated into a composition, e.g., into a liquid medium comprising a hydrogel precursor such as polysaccharide derivative, e.g., an HA derivative, a cellulose derivative, or a dextran derivative, and hence incorporated into a hydrogel made by crosslinking first and second hydrogel precursors (e.g., polysaccharide derivatives or non-polysaccharide polymers). The particles can be, for example, polymeric microparticles or nanoparticles, or liposomes.

Various polymers, e.g., biocompatible polymers, which may be biodegradable, can be used to make the particles. The polymers may be homopolymers, copolymers (including block copolymers), straight, branched-chain, or crosslinked. Suitable biocompatible polymers, a number of which are biodegradable include, for example, poly(lactides), poly(glycolides), poly(lactide-co-glycolides), poly(lactic acids), poly(glycolic acids), poly(lactic acid-co-glycolic acids), polycaprolactone, polycarbonates, polyesteramides, poly(beta-amino ester)s, polyanhydrides, poly(amides), poly(amino acids), polyethylene glycol and derivatives thereof, polyorthoesters, polyacetals, polycyanoacrylates, polyetheresters, poly(dioxanone)s, poly(alkylene alkylates), copolymers of polyethylene glycol and polyorthoesters, biodegradable polyurethanes. Other polymers include poly(ethers) such as poly)ethylene oxide), poly(ethylene glycol), and poly(tetramethylene oxide); vinyl polymers-poly(acrylates) and poly(methacrylates) such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; poly(siloxanes), etc. Other polymeric materials include those based on naturally occurring materials such as polysaccharides (e.g., alginatechitosan, agarose, hyaluronic acid), gelatin, collagen, and/or other proteins, and mixtures and/or modified forms thereof. Chemical or biological derivatives of any of the polymers disclosed herein (e.g., substitutions, addition of chemical groups, for example, alkyl, alkylene, hydroxylations, oxidations, and other modifications routinely made by those skilled in the art) are also encompassed. Furthermore, blends, graft polymers, and copolymers, including block copolymers of any of these polymers can be used. Copolymers can contain various ratios of the different monomeric subunits. For example, a copolymer comprising monomer A and monomer B may contain between 5% and 95% monomer A and between 5% and 95% monomer B (where the percentages refer to the percentage based on number of monomers and add up to 100%). It will be understood that certain of these polymers require use of appropriate initiators or cross-linking agents in order to polymerize.

Additional exemplary polymers include cellulose derivatives such as carboxymethylcellulose, polycarbamates or polyureas, cross-linked poly(vinyl acetate) and the like, ethylene-vinyl ester copolymers, ethylene-vinyl hexanoate copolymer, ethylene-vinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer, or mixtures thereof.

Features such as cross-linking and monomer concentration may be selected to provide a desired rate of degradation of the particles and/or release of a biologically active agent encapsulated or entrapped therein or adsorbed to the surface, etc.

In certain embodiments of the invention the particles are themselves composed of crosslinked polysaccharide derivatives, e.g., HA, dextran, and/or cellulose derivatives.

Microparticles and nanoparticles of use in the invention can have a range of dimensions. Generally, a microparticle will have a diameter of 500 microns or less, e.g., between 1 and 500 microns, between 50 and 500 microns, between 100 and 250 microns, between 20 and 50 microns, between 1 and 20 microns, between 1 and 10 microns, etc., and a nanoparticle will have a diameter of less than 1 micron, e.g., between 10 nm and 100 nm, between 100 nm and 250 nm, between 100 nm and 500 nm, between 250 nm and 500 nm, between 250 nm and 750 nm, between 500 nm and 750 nm. If the particles prepared by any of the above methods have a size range outside of the desired range, the particles can be sized, for example, using a sieve. Particles can be substantially uniform in size (e.g., diameter) or shape or may be heterogeneous in size and/or shape. They may be substantially spherical or may have other shapes, e.g., cylindrical, ellipsoid, or pyramid-shaped, in which case the relevant dimension will be the longest straight dimension rather than the diameter.

Nanoparticles or microparticles can be made using any method known in the art including, but not limited to, spray drying, phase separation, single and double emulsion, solvent evaporation, solvent extraction, and simple and complex coacervation. Particulate polymeric compositions can also be made using granulation, extrusion, and/or spheronization. In certain embodiments of the invention multilayered particles are used. For example, the particles may contain a core and one or more layers coating the core. The core and layer(s) may be made of the same material(s) or different materials. Materials and methods for making particles are described in the literature, for example, in U.S. Pat. No. 4,272,398, which is incorporated herein by reference; U.S. Pat. No. 6,428,815, which is incorporated herein by reference; and references therein.

The conditions used in preparing the particles may be altered to yield particles of a desired size or property (e.g., hydrophobicity, hydrophilicity, external morphology, “stickiness,” shape, etc.). The method of preparing the particle and the conditions (e.g., solvent, temperature, concentration, air flow rate, etc.) used may also depend on the therapeutic agent and/or the composition of the polymer matrix.

Liposomes of use in the present invention can be prepared using any one of a variety of conventional liposome preparatory techniques such as sonication, chelate dialysis, homogenization, solvent infusion coupled with extrusion, freeze-thaw extrusion, microemulsification, as well as others. These techniques, as well as others, are discussed, for example, in U.S. Pat. No. 4,728,578, U.K. Patent Application G.B. 2193095 A, U.S. Pat. Nos. 4,533,254; 4,728,575; 4,737,323; 4,753,788 and 4,935,171; each of which is incorporated herein by reference. See also Gregoriades, G. (ed.), Liposome Technology, vol. 1-3, CRC, Boca Raton, 1984; Gregoriades, G. (ed.), Liposomes as Drug Carriers, John Wiley & Sons, Chichester, 1988, 1984;Lasic, D. D., Liposomes: From Physics to Applications, Elsevier, Amsterdam, 1993; Martin, F. & Lasic, D. (eds.) Stealth Liposomes, CRC, Boca Raton, 1995; Woodle, M. C & Storm, G. (eds.), Long Circulating Liposomes: Old Drugs, New Therapeutics, Springer, Berlin, 1997; Torchilin, V. P. & Weissig, V. (eds.), Liposomes. Practical Approach, Oxford University Press, Oxford, 2003.

Materials which may be utilized in preparing the liposomes of the present invention include any of the materials or combinations thereof known to those skilled in the art as suitable in liposome construction. The lipids used may be of either natural or synthetic origin. Such materials include, but are not limited to, lipids such as cholesterol, phosphatidylcholine, phosphatidylethanolamine, phosphatidylserine, phosphatidylglycerol, phosphatidic acid, phosphatidylinositol, lysolipids, fatty acids, sphingomyelin, glycosphingolipids, glucolipids, glycolipids, sulphatides, lipids with amide, ether, and ester-linked fatty acids, polymerizable lipids, and combinations thereof.

A composition can contain multiple populations of particles, wherein the populations are made of different materials or different ratios of the same materials and/or differ in properties such as size or shape.

The concentration of particles in a solution containing a polysaccharide derivative, e.g., an HA derivative, a cellulose derivative, or a dextran derivative, and the ratio of the weight of the particles to the weight of the polysaccharide derivative, can vary. For example, a solution containing a polysaccharide derivative may contain between 100 μg/ml and 10 g/ml particles, e.g., between 1 mg/ml and 1.0 g/ml, or between 1 mg/ml and 100 mg/ml particles. The ratio of the weight of the particles to the weight of a polysaccharide derivative in a solution can vary, e.g., from 1:100 to 100:1. For example, the ratio can be between 1:10 and 10:1 or between 1:5 and 5:1 or between 1:2 and 2:1, e.g., approximately 1:1.

In certain embodiments of the invention a biologically active agent is physically associated with the particles while in other embodiments of the invention the particles do not have a biologically active agent associated therewith. The physical association can be covalent or noncovalent. The association may be specific or nonspecific. Preferably the physical association is one that remains stable while the particles are manipulated and combined with a polysaccharide derivative in solution and remains stable at least until the composition is administered to a subject and, typically, for a variable and optionally controllable period of time thereafter. The biologically active agent may be associated with the particles through one or more noncovalent interaction mechanisms such as ionic interactions, hydrogen bonds, hydrophobic interactions, etc. For example, one or more agents may be entrapped, embedded, enclosed, or encapsulated within the particles. The particles may be impregnated with the agent and/or the agent may be adsorbed to the surface of the particles. The agent may be released from the particles by diffusion or as the particles degrade in the body. Release may occur over hours, days, weeks, months, etc.

The particles may contain any one or more of the biologically active agent(s) described above, e.g., any of the therapeutic agents described above. In some embodiments of the invention a solution containing a polysaccharide derivative, or a hydrogel formed by crosslinking first and second polysaccharide derivatives, contains at least two distinct populations of particles. The populations may be distinct from one another with respect to any of a number of parameters. For example, the particles may differ in terms of the material(s) from which they are made, the biologically active agent that they contain, their dimensions, etc. For example, in one embodiment the first population includes or consists of particles that contain a first biologically active agent, and the second population includes or consists of particles that contain a second biologically active agent. In certain embodiments of the invention either or both of the polysaccharide derivatives are HA derivatives. In certain embodiments of the invention a first solution contains a first HA derivative and a first population of particles and a second solution contains a second HA derivative and a second population of particles. Crosslinking of the first and second HA derivatives results in formation of a hydrogel that contains first and second populations of particles. In certain embodiments of the invention a first solution contains an HA derivative and a first population of particles and a second solution contains a cellulose derivative and a second population of particles. In certain embodiments of the invention a first solution contains an HA derivative and a first population of particles and a second solution contains a dextran derivative and a second population of particles. Crosslinking of the HA derivative and the celluose or dextran derivative results in formation of a hydrogel that contains first and second populations of particles. In any of these embodiments,the concentrations of the first and second populations of particles in the first and second solutions can be selected to achieve a desired final particle concentration in the hydrogel. In some embodiments of the invention some, but not all, of the particles comprise a biologically active agent. Of course each of the solutions may contain more than one distinct populations of particles, e.g., 2, 3, 4, or more distinct populations of particles.

As will be apparent to one skilled in the art, biologically agents can be loaded into particles during their formation or afterwards. The biologically active agent may be entrapped, embedded, or encapsulated by the polymer matrix or enclosed in the aqueous core of a liposome and/or may coat the surface of the particles. Particles of the biologically active agent may be dispersed throughout the polymer matrix. The biologically active agent preferably constitutes between approximately 0.01% and 90% by weight of the particles, e.g., about 1% to about 80%, or about 10% to about 70% by weight of the particles. In certain embodiments, the biologically active agent comprises approximately 10%-20%, or approximately 30%-50% by weight of the particles. One of skill in the art will understand that in choosing an appropriate polymer and method of manufacture, it is important to select materials and methods that are compatible with stability of the biologically active agent. For example, it may be desirable to avoid processing temperatures that are likely to result in substantial degradation or denaturation of the agent, which may result in loss of bioactivity. It will also be desirable to test the composition so as to ensure that the agent is released in significant amounts over the desired period of time.

In certain embodiments of the invention a biologically active agent is covalently attached to a component of the particles, e.g., to a polymer or lipid component. Any of a variety of methods for forming such covalent attachments can be used. The biologically active agent may include a functional group capable of reacting with a functional group on the polymer or lipid. Either the polymer or lipid, the biologically active agent, or both, may be modified to include a suitable functional group. Alternately, a crosslinking agent can be used. Relevant methods are similar to those described above for covalently attaching a biologically active agent to a polysaccharide derivative. Preferably the attachment does not reduce the biological activity of the agent below useful levels or interfere with particle formation. In certain embodiments of the invention the biologically active agent is covalently attached to a polymer or lipid component of a particle via a bond that is hydrolytically and/or enzymatically labile under physiological conditions. Cleavage of the bond releases or facilitates release of the biologically active agent from the particle.

In certain embodiments of the invention the particles contain one or more additional agents, e.g., excipients, buffers, spheronizing agents, fillers, surfactants, disintegrants, binders, plasticizers, or coatings, in addition to one or more biologically active agent(s). The additional agent may not itself have a significant biological effect but may modulate the biological effect of the biologically active agent. Exemplary materials are described in U.S. Pat. No. 5,846,565, incorporated herein by reference. Suitable agents include, for example, carbohydrates, amino acids, fatty acids, surfactants, salts, metal ions, and bulking agents, and are known to those skilled in the art. The amount of excipient used may be based on the ratio of excipient to the biologically active agent, on a weight basis. For amino acids, fatty acids, salts and carbohydrates, such as sucrose, lactose, mannitol, dextran and heparin, the ratio of carbohydrate to biologically active agent is between about 1:10 and about 20:1 in certain embodiments of the invention. For surfactants, the ratio of surfactant to biologically active agent is between about 1:1000 and about 1:20 in certain embodiments of the invention.

In certain embodiments the additional agent is one that alters the release properties of the biologically active agent from the particles, referred to herein as a “release modulating agent.” The kinetics of release of a biologically active agent from particles that contain a biologically active agent and a release modulating agent differs from the kinetics of release of the biologically active agent from otherwise identical particles that do not contain the release modulating agent. The kinetics may be altered in any of a number of ways. For example, the releasing modulating agent may retard release or increase release. The rate of release may be increased or decreased during part or all of the time period over which release occurs. For example, the presence of the releasing modulating agent may reduce or prevent an initial “burst” effect in which a significant proportion of the biologically active agent is released within a short time following contact of the particles with liquid. Certain particles release a significant fraction of their payload within a desired time period following contact with liquid but may fail to continue releasing additional agent at later time points. The release modulating agent may alter the time required for a specified percentage of the biologically active agent to be released from the particles. For example, the release modulating agent may increase or decrease the time required for release of 10%, 25%, 50%, 75%, 90%, or essentially all of the biologically active agent to be released. For example, the time may be increased or decreased by a factor of between 0.1 and 10-fold. Exemplary release modulating agents include hydrophobic substances, e.g., hydrophobic surfactants such as poloxamers. Other exemplary release modulating agents are phospholipids, cholesterol, polymethacrylates, sugars, proteins, acrylate block copolymers such as Eudagrit E-100 and related compounds, and zinc. The release modulating agent may be one that either forms or fills pores in the polymeric matrix.

The invention further provides a composition comprising (a) microparticles comprising one or more crosslinked polysaccharide derivative(s) (e.g. HA derivative(s)); and (b) a plurality of nanoparticles. The microparticles may be suspended in a medium and applied directly to a location in the body, e.g., to the peritoneum. The nanoparticles may contain a biologically active agent. Thus the composition comprises microparticles comprised of crosslinked polysaccharide derivatives, wherein the microparticles encapsulate nanoparticles. In one embodiment of the invention, the microparticles comprising one or more HA derivative(s) are prepared by sequentially adding and homogenizing solutions of first HA derivative, biologically active agent(s), and second HA derivative in a continuous phase comprising oil and emulsifier. The homogenization is conducted for 1-20 minutes at 1000-9000 rpm. The emulsion is then stirred at 40-50° C. overnight to evaporate water from the dispersed phase. The microparticles are washed with isopropyl alcohol 3-6 times, followed by evaporation of the residual isopropyl alcohol. The biologically active agent can be pre-encapsulated in nanoparticles prior to microencapsulation. The nanoparticles can be polymeric nanoparticles or liposomes.

In another embodiment of the invention, the microparticles can be prepared by spray-drying HA derivatives, biologiclaly active agent(s), and release modulating agent(s).

The invention further provides compositions (hydrogels and solutions comprising a hydrogel precursor and a plurality of particles) in which at least one of the hydrogel precursors is a polysaccharide derivative that comprises a non-polysaccharide polymer portion. The polysaccharide derivative comprising a non-polysaccharide polymer portion can be any of those described in section I. The polysaccharide derivative typically comprises a polysaccharide or derivative thereof covalently attached to one or more non-polysaccharide polymers. The particles can be any of the particles described above.

In yet other embodiments the invention provides a hydrogel comprising a plurality of particles, wherein the hydrogel is formed by crosslinking two non-polysaccharide polymers. The non-polysaccharide polymers are typically dissolved in solution as described above for polysaccharide derivatives, wherein one or both of the solutions contains particles. The solutions contacted with each other, e.g., by administering them to a subject. Each of the non-polysaccharide polymers comprises a functional group, wherein the functional groups are capable of reacting with one another to form covalent bonds. Suitable functional groups are those described above for crosslinking of polysaccharide derivatives. Exemplary non-polysaccharide polymers include those described in Section I that contain or may be modified to contain suitable functional groups for crosslinking. The particles can be any of the particles described above.

It is noted that in any of the embodiments described above, the particles may be provided in a solution containing a hydrogel precursor or may be provided in a separate solution or in dry form.

IV. Applications

The compositions and methods of the invention have a number of uses including, but not limited to, the prevention and treatment of adhesions and the administration of therapeutic agents. For any of these applications, the hydrogel precursors, e.g.,. polysaccharide derivatives, may be provided in dry form (e.g., lyophilized) and may be dissolved in an appropriate liquid medium, e.g., water or saline, prior to administration. Alternately, the hydrogel precursors (e.g., polysaccharide derivatives) may be provided in solution. Biologically active agents and/or particles (optionally comprising one or more biologically active agents) can be added to the dry polymers or solutions in varying amounts, depending on the application, prior to administration. Altnernately, compositions comprising a polysaccharide derivative and a biologically active agent or particles can be provided in dry form and subsequently added to a liquid medium.

A. Modes of Administration

As noted above, the invention provides a variety of methods that comprise administering hydrogel precursors to a location in the body of a subject, wherein the hydrogel precursors become crosslinked to form a hydrogel following administration. The hydrogel precursors are typically administered in solution. The solution(s) can be administered in any of a variety of ways. For example, multiple barrel injection devices (e.g., multiple barrel syringe, dual valve applicator) can be used to deliver multiple solutions to a desired location substantially simultaneously. In certain embodiments of the invention the device comprises a chamber into which the multiple solutions are temporarily ejected and in which mixing can occur prior to administration. The invention encompasses administering hydrogel precursors in separate solutions that contact one another in the body. The invention encompasses administering two or more solutions substantially simultaneously. The two solutions may contact one another during administration. The invention also encompasses administering multiple solutions each comprising a hydrogel precursor by administering a single solution that is formed from the multiple solutions. The solutions will typically be combined shortly before administration such that little or no crosslinking will occur during administration and the compositions will remain in a fluid state. For example, in a method of inhibiting adhesions comprising the steps of: (a) administering a first hydrogel precursor comprising a first polysaccharide derivative to a location within the body of a subject and (b) administering a second hydrogel precursor comprising a second polysaccharide derivative or a non-polysaccharide polymer to the location within the body of the subject, the invention includes embodiments in which the hydrogel precursors are dissolved in solutions that are contacted with one another and/or mixed with one another shortly before administration. The invention also includes embodiments in which the hydrogel precursors are dissolved in separate solutions that are administered substantially simultaneously over one or more discrete or consecutive time period of about 1-60 seconds, e.g. over 1-30 seconds, 5-20 seconds, about 10 seconds, etc. The invention also includes embodiments in which the hydrogel precursors are administered in separate solutions that contact one another after administration. Solutions can be administered substantially simultaneously by a variety of methods such as by co-extruding them from barrels of a multiple barrel injection device. In embodiments of the invention in which three or more compositions are administered, any two or more of the compositions can be administered substantially simultaneously. Compositions can be administered over a single time period or over multiple discrete time periods, each of which may be considered a separate administration. The multiple discrete time periods may take place over minutes, hours, etc. For example, pan-peritoneal administration or complex spinal or cranial surgery may involve multiple discrete administrations over a period of hours.

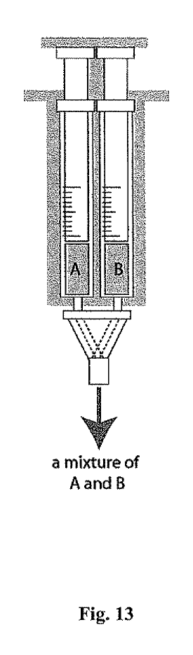

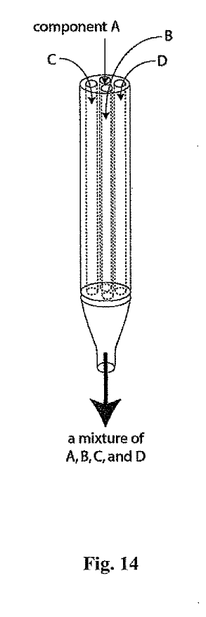

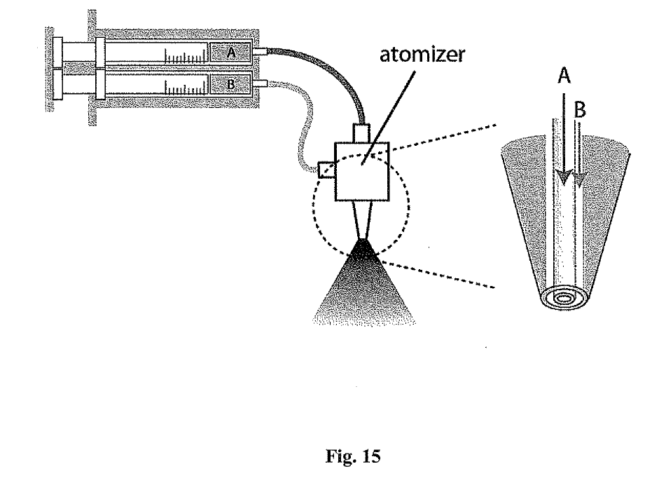

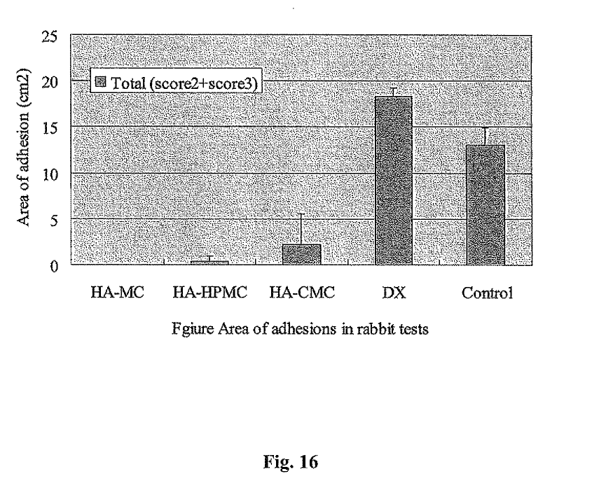

FIG. 13 shows an exemplary device of use for administering solutions. FIG. 14 shows a multi-channel device that is useful for administering up to four different components, e.g., four different solutions. Some of the solutions contain a hydrogel precursor, e.g., a polysaccharide derivative, while other solutions need not contain a hydrogel precursor. For example, some of the solutions may contain particles. Alternately, particles may be loaded into a channel of the device in the absence of a liquid and mixed with the solution(s) during administration. FIG. 15 shows a double barreled device attached to a droplet forming device such as a nebulizer or atomizer. The right portion of the figure is an enlargement of a portion of the device. Such devices may be of particular use to rapidly administer a composition to a relatively large area, e.g., for pan-peritoneal application.

Alternately, individual injection devices (syringes, catheters, cannula, etc.) can be used, provided that care is taken to allow the solutions to contact one another as they are administered or shortly thereafter. Endoscopes, e.g., laparoscopes, arthroscopes, etc., can be used. For example, scopes that have multiple channels are suitable. In one embodiment, a double injection laparoscope or arthroscope is used. In one embodiment, the compositions are applied under imaging guidance, e.g., fluoroscopic guidance. In another embodiment, the first and second solutions can simply be mixed in a vessel and then either poured, sprayed, or squirted onto a desired location. Solutions can be mixed with a mixing implement or spread with a spreading implement, e.g., a spatula-like implement, after administration.

In certain embodiments of the invention a solution comprising a hydrogel precursor, e.g., a polysaccharide derivative such asan HA derivative, comprises a detectable substance. The substance may be visually detectable, e.g., a dye or colorant, or may be detectable by another means (e.g., the substance may be radioactive). Preferably the detectable substance is not harmful to the body (unless it is a substance such as an anti-proliferative or anti-neoplastic agent whose mechanism of action entails toxicity to normal as well as undesired cells). The presence of the detectable substance allows the individual adminstering the composition to more readily identify the administered material and is therefore of use to guide administration. Certain detectable substances may be used to track the polysaccharide derivatives after they have been administered, e.g., to monitor their degradation.

B. Prevention and Treatment of Adhesions

The compositions of the invention may be administered to treat or prevent adhesions in the context of any of a wide variety of surgery types. Nonlimiting examples of surgical procedures in which the compositions and methods of the invention are of use include abdominopelvic, ophthalmic, orthopedic, gastrointestinal, thoracic, cranial, head and neck, cardiovascular, gynecological, joint (e.g., arthroscopic), urologic, plastic, reconstructive, musculoskeletal, and neuromuscular surgeries. Specific abdominal procedures include, e.g., surgeries to remove or repair one or more abdominopelvic organs or a portion thereof. Examples include surgery on the stomach, intestines (e.g., duodenum, jejunum, ileum, colon, rectum), appendix, gall bladder, liver, kidney, bladder, urethra, and prostate. Abdominopelvic surgeries also include hernial repair, aneurysm repair, and lysis of peritoneal adhesions. Gynecological procedures include surgeries to treat infertility due to tubal disease, e.g., with adhesions attached to ovaries, fallopian tubes and fimbriae. Such surgeries including salpingostomy, salpingolysis and ovariolysis. Gynecological surgeries include ovariectomy, hysterectomy, removal of endometriosis, preventing de novo adhesion formation, treatment of ectopic pregnancy, myomectomy of uterus or fundus, etc. Additional surgeries include surgeries to treat incontinence or vaginal prolapse. Obstetric surgeries include Caesarean section. Musculoskeletal surgeries include lumbar laminectomy, lumbar discectomy, flexor tendon surgery, spinal fusion, and joint replacement or repair. Neuromuscular surgeries include those undertaken to repair nerves, ablate nerves, or free entrapped nerves. Thoracic surgeries which involve stemotomy can result in adhesion formation between the heart or aorta and the epithelial layer lining of the thoracic cavity. Thoracic surgeries include surgery on the esophagus, lung surgery (e.g., lung reduction or removal of tumors), bypass surgery, aneurysm repair, and heart valve replacement. Surgeries also include those performed to implant any of a variety of prostheses or implantable devices such as defibrillators and those performed for diagnostic purposes. Cranial surgical procedures include surgery for tumors, epilepsy, require more than one procedure. These procedures often result in adhesions involving the skull, meninges, and/or cortex. Ocular surgical uses include surgery for strabismus, glaucoma filtering surgery, and lacrimal drainage system procedures.

Thus in certain embodiments of the invention the compositions are administered to treat or prevent peritoneal adhesions. In other embodiments of the invention the compositions are administered to treat or prevent pleural adhesions, e.g., fibrous adhesions between the lobes of the lung and/or between the visceral and the parietal pleura. In other embodiments of the invention the compositions are administered to treat or prevent adhesions involving the pericardium (e.g., epicaridium, visceral or parietal pericardium, fibrous pericardium). In yet other embodiments of the invention the compositions are adminstered to treat or prevent epidural adhesions (adhesions in the epidural space, involving the dura).

Typically the compositions will be administered at some point between the time the first incision is made and the time at which surgical closure has been completed or the last endoscopic instrument has been withdrawn from the subject's body, whichever occurs later. The compositions may be administered to a subject who has not previously developed adhesions or may be administered to a subject who has developed adhesions. In one embodiment the compositions are administered to a subject who is undergoing a procedure to reduce pre-existing adhesions. For example, the procedure may entail mechanical or chemical lysis or disruption of pre-existing adhesions, followed by application of a composition of this invention to prevent recurrence of adhesions.

The total volume of composition administered to a subject can vary based on a variety of factors, primarily the area intended to be covered by a hydrogel layer, the thickness of hydrogel desired, the site of administration, and whether the composition comprises a therapeutic agent. Exemplary volumes range between 0.1 ml and 5000 ml, e.g., between between 0.5 ml and 1000 ml, between 1 ml and 500 ml, between 10 ml and 100 ml, etc., it being understood that these volumes are approximate and that all subranges are included.

The compositions may be administered so that the administered material, or a hydrogel formed therefrom, covers a site of damage, e.g., a surgical incision or injury or so as to cover a portion of the epithelial surface, e.g., peritoneum, pleura, dura, located opposite to a site of damage. The area covered may surround and extend outwards for a variable distance from the site of damage or from a region located on a tissue located opposite from the site of damage. For example, the area covered may extend outwards from the site of damage for an average distance of up to about 1, 2, 3, 4, 5, or more cm. The total area covered by the administered material or a hydrogel formed therefrom may range from approximately 0.1 cm² to about the total area of the peritoneum, e.g., up to approximately 1.5-2.0 m². For example, the total area covered may range from approximately 1.0 cm² to approximately 1.0 m², e.g., from approximately 5.0 cm² to approximately 0.5 m². The composition may be applied to a contiguous area or to multiple discrete areas separated by regions that are not covered.

In the case of compositions that comprise particles, the volume and weight of particles delivered can also vary and will depend on the total volume of solution(s) administered and the concentration of particles in the solution(s). These parameters can be adjusted to deliver a desired total particle volume or weight. In certain embodiments of the invention the particles are delivered in an amount between 1 mg/kg and 2 g/kg to a subject. Exemplary amounts range between 1 mg/kg and 1000 mg/kg, e.g., between 5 mg/kg and 700 mg/kg. The amount of particles delivered need not be based on the weight of the subject but may instead be expressed in terms of absolute amount. Exemplary amounts range between 1 mg and 500 g, e.g., between 1 mg and 100 g or between 10 mg and 1 g.

C. Drug Delivery

Compositions in which either the hydrogel formed as described above contains a therapeutic agent (optionally physically associated with particles) may be used for the treatment of a wide variety of diseases, disorders, and conditions, or for prophylactic purposes. In certain embodiments of the invention the hydrogel provides sustained release of the therapeutic agent. A single administration of the hydrogel precursors and agent may provide an effective concentration of the agent over a time period at least 2, 3, 5, 10, 20, or more times as long as would result if the same amount of the agent was administered in the absence of the hydrogel precursors and/or use of the hydrogel system allows a greater total dose to be administered without causing undue side effects. In certain embodiments of the invention the rate of release is controlled by controlling the rate at which the hydrogel and/or the particles degrade. The invention therefore provides a composition comprising a hydrogel precursor in solution and a therapeutic agent, wherein the hydrogel precursor is any of the hydrogel precursors described herein and is provided at any of the concentration ranges described herein.

The compositions may be administered to any location within the body of a subject including, but not limited to, the locations discussed above in the context of inhibiting adhesions. For example, the compositions may be admininistered to the peritoneum to treat a disease or disorder with manifestations primarily within the abdominopelvic cavity or to treat a systemic disease. Peritoneal drug delivery is an attractive option for a variety of therapeutic agents for treatment of systemic diseases, due at least in part to the large surface area of the peritoneum available for absorption of the agent. In certain embodiments of the invention a composition comprising an anti-infective agent is used to treat infections or prophylactically, e.g., to reduce the likelihood of infection following surgery. Patients who undergo surgical procedures in which the intestinal wall may be compromised are at particular risk of infection, e.g., peritonitis. In one embodiment the composition is administered to the abdominopelvic cavity of a subject during abdominal surgery or thereafter. The composition may be applied anywhere within the abdominopelvic cavity, either directly to one or more abdominal organs and/or adjacent tissues or to parietal peritoneum located approximately opposite to an abdominal organ so as to form a hydrogel that separates the visceral peritoneum covering the organ from the parietal peritoneum. The compositions may be administered using extended local peritoneal administration, or pan-peritoneally.

In another embodiment, a composition of the invention is used to administer an anti-neoplastic agent. The anti-neoplastic agent may be administered to treat a tumor located in the abdominopelvic cavity, e.g., a tumor of an abdominal or pelvic organ. In one embodiment, the composition is administered to a subject prior to, during, or after surgery to remove a tumor located in the abdominopelvic cavity. The composition may be administered pan-peritoneally. Without wishing to be bound by any theory, administering a composition of the invention may reduce the development of metastases and/or inhibit peritoneal seeding of a tumor. In another embodiment, a composition of the invention is administered to a subject suffering from a tumor in the abdominopelvic cavity. The composition provides sustained release of an anti-neoplastic agent. For example, the agent may be released over a time period of at least 1, 2, 3, 4, 6, or 8 weeks, or longer. The composition may be applied to an organ in which the tumor is located or may be applied more widely. Thus the invention provides a method of treating a subject suffering from or at risk of developing a tumor comprising administering a composition of the invention to the subject, wherein the composition comprises an anti-neoplastic agent. Any of the compositions comprising at least one hydrogel precursor, e.g., a polysaccharide derivative such as an HA derivative described herein may be employed, wherein the at least one hydrogel precursor becomes crosslinked to form a hydrogel following administration to a subject. The therapeutic agent may be physically associated with particles of any of the types described herein. The tumor may be located in the abdominopelvic cavity.

In yet another embodiment a composition of the invention is used to administer a therapeutic agent to a joint of a subject. The subject may, for example, suffer from arthritis. Exemplary therapeutic agents suitable for administration to the joint space include anti-inflammatory agents and analgesic agents. Thus the invention provides a method of treating a subject suffering from or at risk of developing a disease or condition that affects a joint comprising administering a composition of the invention to the joint, wherein the composition comprises a therapeutic agent selected to treat or prevent the condition. The composition may, for example, be injected into the synovial cavity. Any of the compositions comprising at least one hydrogel precursor described herein may be employed, wherein the at least one hydrogel precursor becomes crosslinked with another hydrogel precursor to form a hydrogel following administration to a subject. The therapeutic agent may be physically associated with particles of any of the types described herein.

Compositions may be administered using any of a variety of routes e.g., intradermal, subcutaneous, intramuscular, etc. Any body tissue can be used as a depot for a composition comprising one or more hydrogel precursors, e.g., polysaccharide derivatives and a plurality of particles, wherein the hydrogel precursor(s) become crosslinked to form a hydrogel following administration.

D. Rapid Hydrogel Formation

As noted above, one aspect of the invention is the recognition of the advantages afforded by rapidly crosslinking hydrogel precursors to form a hydrogel in situ and the development of suitable compositions and methods by which to achieve rapid in situ hydrogel formation. Any of the embodiments of the invention may be practiced with compositions containing hydrogel precursors that form hydrogels within between 1-5 and 60 seconds, between 1-5 and 30 seconds, between 1-15 and 20 seconds, or between 1-5 and 10 seconds following contact of the hydrogel precursors with one another. Any of the embodiments of the invention may be practiced with compositions containing hydrogel precursors that form hydrogels within between 1-5 and 60 seconds, between 1-5 and 30 seconds, between 1-15 and 20 seconds, or between 1-5 and 10 seconds following contact of solutions containing the hydrogel precursors with one another. Any of the embodiments of the invention may be practiced with compositions containing hydrogel precursors that form hydrogels within between 1-5 and 60 seconds, between 1-5 and 30 seconds, between 1-15 and 20 seconds, or between 1-5 and 10 seconds following administration of the hydrogel precursors to a subject.

V. Packages or Kits

The invention also provides packages or kits, comprising one or more compositions as described herein in a container. For example, the container may include an HA derivative in dry (e.g., lyophilized) form or in solution. If the HA derivative is provided in dry form, the product package may include a container with an appropriate solvent or diluent, e.g., sterile water for injection. The package can also include a notice associated with the container, typically in a form prescribed by a government agency regulating the manufacture, use, or sale of medical devices, pharmaceuticals, and/or biopharmaceuticals, whereby the notice is reflective of approval by the agency of the compositions, for human or veterinary administration to treat adhesions diseases or for one or more indications in addition to, or instead, of for treating adhesions (e.g., as a prophylaxis for or treatment of post-surgical infection). Instructions for the use of the agents or composition may also be included. Such instructions may include information relating to the reconstitution of an HA solution, the addition of particles thereto, the loading of a delivery device, the appropriate amounts and modes of administration, etc.

In certain embodiments of the invention the package will contain multiple individual containers, each containing an HA derivative either in dry form or in solution. For example, a first container contains a first HA derivative and a second container contains a second HA derivative. The first and second HA derivatives may be provided in predetermined amounts such that when contacted with each other in solution they form a hydrogel having desired characteristics. The package may also include one or more containers containing biologically active agent(s) to be combined with the HA derivative prior to administration.

In certain embodiments, the package contains other polysaccharide derivatives such as cellulose or dextran derivatives. In certain embodiments, the package includes a combination of HA, cellulose, and/or dextran derivatives for use in forming the desired hydrogel. The package may also include other polymers such as synthetic polymers. The package may also include a protein.

The pharmaceutical package may also include a receptacle containing particles to be included in solution with a polysaccharide derivative, e.g., an HA, cellulose, or dextran derivative. Alternately, the receptacle may contain the derivative and particles, e.g., in a predetermined ratio. The particles may contain a biologically active agent.

The multiple containers may be provided in a single larger container, e.g., a plastic or styrofoam box, in relatively close confinement.

The package may include a device or receptacle for preparation of a solution containing a polysaccharide derivative, e.g., an HA, cellulose, or dextran derivative. The device may be, e.g., a measuring or mixing device.

The package may include a device for administering a composition of the invention. Exemplary devices include syringes, e.g., multiple barrel syringes, catheters, endoscopes, arthroscopes, laparoscopes. The endoscope, arthroscope, or laparoscope may have multiple channels to allow adminstration of multiple individual solutions. Other devices that may be included are attachments for endoscopic or laparoscopic instruments that allow for convenient administration of a composition of the invention. Of course such devices can also be provided separately.

### EXAMPLES

### Example 1

### Preparation and Characterization of Hyaluronic Acid Derivatives and Cross-Linked Hydrogels

Materials and Methods

Hyaluronic acids: Hyaluronic acids (HA, nominal 1.36 MD: high MW and 490 kD: medium MW) were purchased from Genzyme Corporation (Cambridge, Mass.). HA (nominal 50 kD: low MW) was purchased from Lifecore Biomedical, Inc. (Chaska, Minn.). All other reagents were purchased from Sigma-Aldrich (St. Louis, Mo., USA).

Preparation of cross-linkable hyaluronic acids: In situ cross-linkable HA derivatives were synthesized following a previously reported method (Jia X, Colombo G, Padera R. Langer R. Kohane D S. Prolongation of sciatic nerve blockade by in situ cross-linked hyaluronic acid. Biomaterials 2004;25(19):4797-4804, which is incorporated herein by reference). Briefly, HA-adipic dihydrazide (HA-ADH) was prepared by reacting HA (medium MW unless specified otherwise) with a 30-fold molar excess of adipic dihydrazide in the presence of 1-ethyl-3-carbodiimide (EDC) and 1-hydroxybenzotriazole (HOBt) at pH 6.8 and room temperature. The product was purified by exhaustive dialysis and ethanol precipitation. HA-aldehyde (HA-CHO) was prepared by reacting HA (high MW unless specified otherwise) with an equi-molar sodium periodate for 2 hours at room temperature in the dark. The reaction was terminated by adding ethylene glycol. The product was purified by exhaustive dialysis. The purified products were lyophilized and stored at 4° C. Molecular weights (MWs) of cross-linkable HA derivatives were determined using gel permeation chromatography (GPC). GPC was performed with Ultrahydrogel Linear column (Waters, Milford, Mass.) and 0.05M acetate aqueous solution containing 0.2M NaCl (pH 6.7) as a mobile phase (0.8 ml/min). A MW calibration curve was prepared with a series of pullulan standards. The degrees of modification were determined as per the reported methods using ¹H NMR analysis (Varian Mercury 300 MHz, for HA-ADH) (Jia X, Colombo G, Padera R, Langer R, Kohane D S. Prolongation of sciatic nerve blockade by in situ cross-linked hyaluronic acid. Biomaterials 2004;25(19):4797-4804, which is incorporated herein by reference) and the aldehyde assay (for HA-CHO) (Kohane D S, Lipp M, Kinney R C, Anthony D C, Louis D N, Lotan N, et al. Biocompatibility of lipid-protein-sugar particles containing bupivacaine in the epineurium. J Biomed Mater Res 2002;59(3):450-459, which is incorporated herein by reference).

Characterization of HAX hydrogels: In situ gelation time of the hydrogel was measured as follows. A magnetic stirring bar (Teflon fluorocarbon resin, 5×2 mm, Fisher Scientific) was placed in the center of a hundred μl droplet of HA-ADH solution in saline (20 mg/ml) in a Petri dish. A hundred μl of HA-CHO solution (20 mg/ml) was then added to the HA-ADH drop, and the solution was stirred at 155 rpm using a Coming model PC-320 hot plate/stirrer. The gelation time was considered to be the time when the solution formed a solid globule, which completely separated from the bottom of the dish. The results are reported as averages and standard deviations of 4 independent measurements. Morphology of the lyophilized HAX gel was observed by scanning electron microscopy (JEOL JSM 6060, JEOL USA, Inc., Peabody, Mass.). The lyophilized gel was fractured after cooling in liquid nitrogen to expose the structures inside the gel. The fractured sample was sputter-coated with palladium and gold (100 Å thick) prior to observation.

Statistical analysis for Examples 1-5. As the numerical data did not always follow a normal distribution, they were expressed as medians with 25^{th} and 75^{th} percentiles. Statistical inferences were made using Mann-Whitney U-tests, Kruskal-Wallis tests, or Fisher's exact test, using SPSS software (Chicago, Ill.). A p-value<0.05 on a 2-tailed test was considered statistically significant.

Results

Cross-linkable HA derivatives HA-adipic dihydrazide (HA-ADH) and HA-aldehyde (HA-CHO) were characterized by a variety of different methods. The MWs of original and modified HAs are summarized in Table 1. The peak average MW (Mp) increased slightly and polydispersity of the polymer increased more than twice after ADH modification. Aldehyde (CHO) modification resulted in a significant reduction in MW. After the CHO modification, the MW (Mp) of HA (high MW) and HA (medium MW) decreased from nominal 1.36 MD and 478 kD to 188 and 253 kD, respectively, which is consistent with a previous report (Jia X, Colombo G, Padera R, Langer R, Kohane D S. Prolongation of sciatic nerve blockade by in situ cross-linked hyaluronic acid. Biomaterials 2004;25(19):4797-4804, which is incorporated herein by referfence.) This result indicated that the 2 hours of oxidation reaction induced significant sugar ring cleavage, irrespective of the original MW. ¹H NMR and the aldehyde assay results indicated 52.9±4.7% conjugation of ADH to HA (n=4) and formation of 16.6±4.8% aldehyde groups in each repeating unit of HA (n=7), respectively.

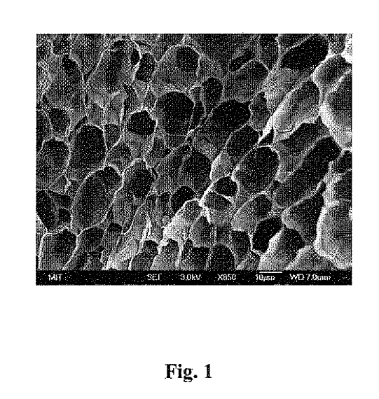

The HAX gel formed quickly upon contact of the two HA derivatives. Under constant stirring of the two components as described in Methods, the HAX gels (20 mg/ml) formed in 3.5±0.6 sec. The morphology of the cross-linked HAX gel was examined with SEM after lyophilization (FIG. 1). The cross-linked hydrogel had continuous circular or polygonal pores, typical of cross-linked hydrogels (Jia X, Burdick J A, Kobler J, Clifton R J, Rosowski J J, Zeitels S M, et al Synthesis and Characterization of in Situ Cross-Linkable Hyaluronic Acid-Based Hydrogels with Potential Application for Vocal Fold Regeneration. Macromolecules 2004;37(9):3239-3248, which is incorporated herein by reference) with a diameter of 10-20 μm.

TABLE 1
Summary of molecular weights of original and modified HAs

| | Nominal¹ | Mp² | Mw³ | Mn⁴ | PDI⁵ |
|---|---|---|---|---|---|
| HA, high MW | 1,360 kD | — | — | — | — |
| HA, medium MW | 490 kD | 478 kD | 1,432 kD | 276 kD | 5.2 |
| HA, low MW | 50 kD | 139 kD | 178 kD | 74 kD | 2.4 |
| HA-ADH | — | 551 kD | 1,502 kD | 108 kD | 13.9 |
| (from HA, | | | | | |
| medium MW) | | | | | |
| HA-ADH | — | 141 kD | 296 kD | 67 kD | 4.4 |
| (from HA, | | | | | |
| low MW) | | | | | |
| HA-CHO | — | 188 kD | 214 kD | 62 kD | 3.4 |
| (from HA, | | | | | |
| high MW) | | | | | |
| HA-CHO | — | 253 kD | 266 kD | 43 kD | 6.1 |
| (from HA, | | | | | |
| medium MW) | | | | | |

¹Provided by manufacturers
²Peak average molecular weight
³Weight average molecular weight
⁴Number average molecular weight
⁵Polydispersity index = Mw/Mn

### Example 2

### Biocompatibility of HAX Hydrogels in Vitro

Materials and Methods

In vitro cell viability assay: Human mesothelial cells (ATCC, CRL-9444) were cultured in Medium 199, containing Earle's salts, L-glutamine, and 2.2 g/L sodium bicarbonate and supplemented with 3.3 nM epidermal growth factor, 400 nM hydrocortisone, 870 nM insulin, 20 mM HEPES, and 10% fetal bovine serum. Cells from passage 5 through 25 were used for the following studies. Mesothelial cells were seeded into 24-well plates at a density of 50,000 cells per well in 1 ml of culture medium. After overnight incubation, the culture medium was replaced with fresh medium or fresh mediums with 10 U/ml hyaluronidase, and a 100 μl cylindrical cylindrical (diameter: 5 mm, height: 5.1 mm) HAX gel (20 mg/ml) was prepared sterilely and added to each well. After varying periods of incubation in the presence of the hydrogels, cell viability was assessed with an MTT assay kit (Promega CellTiter 96 Non-Radioactive Cell Proliferation Assay). Results were reported as medians with 25^{th} and 75^{th} percentiles of the measured absorbance normalized to the absorbance of non-treated control cells (% normalized cell viability=100×Absorbance for cells grown in the presence of a sample in medium/absorbance for cells grown in medium).

Results

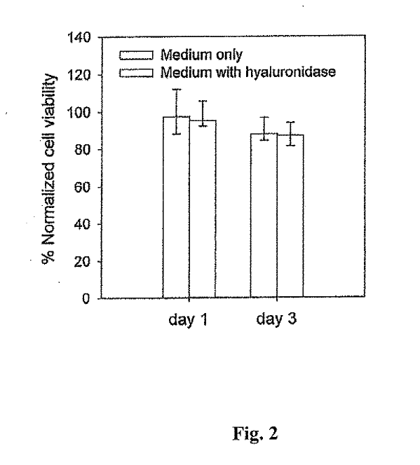

We determined the in vitro cytotoxicity of HAX gel on mesothelial cells (diZerega G S. Peritoneum, peritoneal healing, and adhesion formation. In: diZerega G S, editor. Peritoneal Surgery. New York: Springer, 2000. p. 3-37, which is incorporated herein by reference). These were grown in the presence of 20 mg/ml HAX 100 μl gels (see Methods) for up to 3 days in medium with or without 10 units/ml of hyaluronidase, which degrades HAX gels. The presence of HAX had no statistically significant effect on cell viability in any group as assessed with the MTT assay (FIG. 2), except for a minor (16%) decrease in viability in cells exposed to HAX in the presence of hyaluronidase after 3 days (p=0.042). The in vitro cell proliferation assays indicated that the HAX gel was compatible with mesothelial cells in both non-degrading condition (plain medium) and degrading condition (medium containing 10 U/ml hyaluronidase).

### Example 3

### Prevention of Peritoneal Adhesions by In Situ Cross-Linked HAX Gel

Materials and Methods

In vivo application of HAX gel. Animals were cared for in compliance with protocols approved by the Massachusetts Institute of Technology Committee on Animal Care, in conformity with the NIH guidelines for the care and use of laboratory animals (NIH publication #85-23, revised 1985). Female albino rabbits (Oryctolagus cuniculus; New Zealand White, Covance, Hazleton, Pa.) (3±0.5 kg) were used as model animals. Anesthesia was induced using Ketamine (35 mg/kg i.m.) and Xylazine (5 mg/kg i.m.); maintenance was achieved using 1-3% isoflurane in oxygen administered via endotracheal tube. Aseptic technique was used throughout. The animals were provided with lactated Ringer's solution throughout the surgery and the vital signs were monitored continuously. A 10 cm long midline incision was made along the linea alba on the abdominal wall, and the peritoneum was opened. Peritoneal adhesions were induced according to a method reported in the literature (Orita H, Fukasawa M, Girgis W, diZerega G S. Inhibition of postsurgical adhesions in a standardized rabbit model: intraperitoneal treatment with tissue plasminogen activator. Int J Fertil 1991;36(3): 172-177, which is incorporated herein by reference) with modification. On the right lateral abdominal wall, a 3×4 cm defect comprising the parietal peritoneum and a layer of muscle (˜1 mm thick) was excised starting 1 cm from the midline. Subsequently, the cecum was externalized seven haustra (beginning the 6^{th} haustra distal to the ileocecal junction to the 12^{th} haustra) on the anti-mesenteric side were isolated and abraded bidirectionally for 80-160 strokes using a sterile surgical brush resulting in bleeding.

Twenty animals were assigned randomly to experimental groups: (i) no treatment (n=12); (ii) covering the excised abdominal wall and abraded cecal surface with 10 ml of cross-linked HA (n=8). Prior to application, the materials were sterilized by germicidal UV illumination for 2 hours and dissolved in sterile saline. The gel precursor solutions (5 ml of HA-ADH (20 mg/ml) and 5 ml of HA-CHO (20 mg/ml)) were placed in separate sterile 10-ml syringes, which were connected to a Baxter dual valve applicator, and co-extruded through a 15-gauge needle. The liquid precursors started to gel instantly, conforming to the shape of the applied area. To visual exam, gelation was complete in less than 3 minutes: the hydrogel did not flow beyond that point.

After treatment of the injured areas, the peritoneum and abdominal wall were closed with 2-0 Ethilon and 3-0 Dexon, respectively. The skin was closed with 2-0 Ethilon. Animals were allowed to awaken and had food and water ad libitum. Buprenorphine 0.02-0.03 mg/kg was administered sc 8 hours post-surgery.

One week after the procedure, animals were euthanized with sodium pentobarbital 100 mg/kg IV. Adhesions were scored using a modification of a reported method (Burns J W, Skinner K, Colt J, Sheidlin A, Bronson R, Yaacobi Y, et al. Prevention of tissue injury and postsurgical adhesions by precoating tissues with hyaluronic acid solutions. Journal of Surgical Research 1995;59:644-652, which is incorporated herein by reference). Score 0=no adhesion, score 1=tissue adherence that would separate with gravity, score 2=tissue adherence separable by blunt dissection, score 3=adhesion requiring sharp dissection. Tissues recovered from the necropsy were fixed in 10% formalin, embedded in paraffin, sectioned, and stained with hematoxylin and eosin for histological examination using standard techniques.

Results

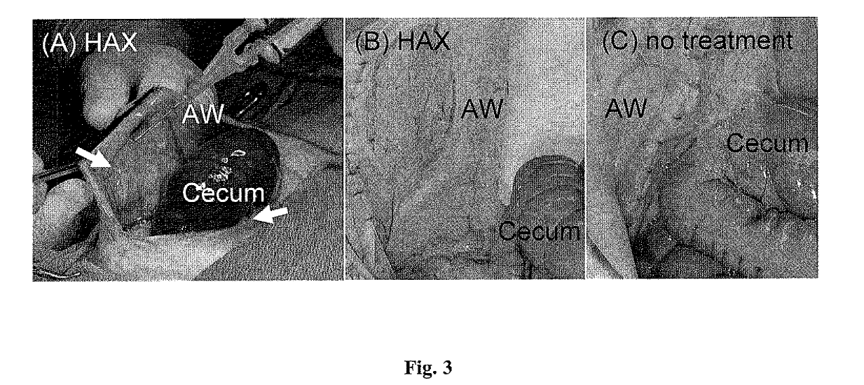

Twenty animals received cecal abrasion and partial abdominal wall excisions as described in Methods. The twelve animals that were not treated with HAX gel (controls) lost 5.7±4.6% of body weight during the first week after the surgery. Ten out of 12 (83%) rabbits developed score 3 adhesions (FIG. 3C). Among those 10 rabbits, 6 developed adhesions directly apposed to the abdominal wall excision, 2 developed adhesions involving the edge of the excision, and 1 developed adhesions involving multiple bowel segments; in the latter animal, there was significant local bleeding during the surgery. One rabbit developed an adhesion to the midline abdominal sutures, i.e., that was independent of the excised abdominal wall.

The eight animals that were treated with 10 ml HAX gel lost 9.0±7.4% of body weights during the first week after the surgery (p=not significant compared to untreated controls). Only 2 animals (25%) developed score 3 adhesions (p=0.019 compared to untreated controls, Fisher's exact test). Four out of 8 animals showed no adhesions (FIG. 3B). One animal had an adhesion which separated with gravity (score 1). Two animals developed adhesions (scores 2 and 3) on the abdominal suture site, and 1 animal developed a score 3 adhesion between abraded haustra and the non-abraded ileocecal junction. However, none of them involved injured abdominal walls. Note that the score 2 and one score 3 adhesion involved suture sites, where HAX gel was not applied. Similarly, the other score 3 adhesion between two loops of bowel also involved only one treated surface. At the time of dissection, HAX gel material was still present on the treated sites (injured abdominal wall and/or abraded cecum surface). To gross inspection, the quantity and elasticity of the material were significantly reduced.

From the data from the untreated animals, it would appear that injury to the abdominal wall played a critical role in developing adhesions. The effect of abrasion of the cecal surface seemed to be less significant. Abraded cecal surfaces that were not involved in adhesions healed in one week without leaving any noticeable mark, and adhesions often involved both abraded and non-abraded haustra. Furthermore, in several pilot studies where the cecum was abraded but the abdominal wall was not injured or only mildly abraded, the incidence of adhesions was very low (data not shown).

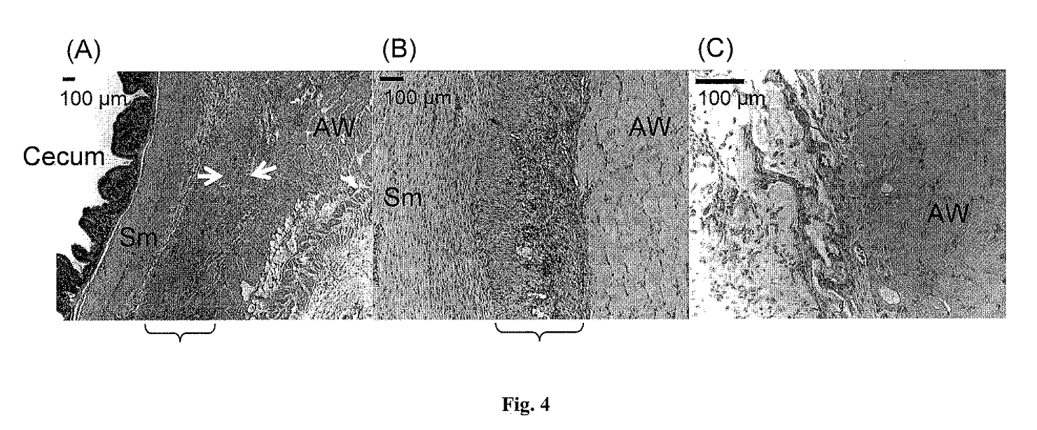

On light microscopy, samples taken from the adhesion sites in untreated animals showed close apposition of the muscular layers of the bowel to the abdominal wall musculature, with varying thicknesses of intervening inflammation and fibrosis, as well as evidence of muscular injury (edema, small dark-staining cells, centralized nuclei) (FIGS. 4A and 4B). In contrast, samples taken from injury sites in treated animals without adhesions showed a coating of a bluish staining material, with a mild-to-moderate infiltrate of inflammatory cells, mostly macrophages but with some neutrophils (FIG. 4C). Samples from adhesion sites in the two animals treated with HAX that developed adhesions had the same appearance as adhesion sites from untreated animals. Here, no coating was observed in samples from those two animals; it should be noted that those adhesions happened at sites that had not been coated.

The results of this experiment are summarized in Table 2. The results demonstrate the in vivo efficacy of HAX gels in reducing peritoneal adhesions. The ability of the gels to prevent adhesions was particularly clear when comparing the prevalence of score 3 adhesions, which were firm links that could only be separated by cutting; lower-grade adhesions were different grades of stickiness rather than adhesions in the surgically-relevant sense. Ten out of 12 animals (83%) in the non-treated control group developed score 3 adhesions within 7 days post-surgery. Histological examination confirmed that the score 3 adhesions were consequences of inflammation and fibrosis. In contrast, score 3 adhesions only occurred in 2 of 8 animals (25%) treated with HAX gels applied on the injured sites. Both of the score 3 adhesions seen in the treatment group involved either the sutured incision or between two cecal surfaces, which were not treated with HAX. If adhesions to uncoated areas are excluded from the analysis, the incidences of score 3 adhesions in the control and treated groups are 82% (9 of 11) and 0% (0 of 6) respectively.

Notably, the analysis demonstrated that our cross-linked HAX gels are biocompatible in the peritoneum. We have previously shown that this system is biocompatible in the perineurium (Jia X, Colombo G, Padera R, Langer R, Kohane D S. Prolongation of sciatic nerve blockade by in situ cross-linked hyaluronic acid. Biomaterials 2004;25(19):4797-4804, which is incorporated herein by reference). However, biocompatibility in one tissue does not necessarily predict biocompatibility in the peritoneum. For example, although polymeric microspheres are biocompatible in the perineurium (Kohane D S, Lipp M, Kinney R C, Anthony D C, Louis D N, Lotan N, et al. Biocompatibility of lipid-protein-sugar particles containing bupivacaine in the epineurium. J Biomed Mater Res 2002;59(3):450-459, which is incorporated herein by reference) and many other tissues, they tend to cause adhesions in the peritoneum (Kohane D S, Tse J Y, Yeo Y, Padera R, Shubina M, Langer R. Biodegradable polymeric microspheres and nanospheres for drug delivery in the peritoneum. J Biomed Mater Res 2005:In press, which is incorporated herein by reference).

TABLE 2
Evaluation of peritoneal adhesions

| | No treatment (n = 12) | | HAX (n = 8) | |
|---|---|---|---|---|
| % weight change | −5.7 ± 4.6 | | −9.0 ± 7.4 | |
| | Frequency | Percentage | Frequency | Percentage |
| Score 3 | 10 | 83 | 2 | 35 |
| Score 2 | 0 | 0 | 1 | 12.5 |
| Score 1 | 0 | 0 | 1 | 12.5 |
| No adhesion | 2 | 17 | 4 | 50 |

| | | |
|---|---|---|
| Median adhesion score | 3 (3-3) | 0.5 (0-2.25) |

### Example 4

### Effects of HA Degradation Products on tPA and PAI-1 Production

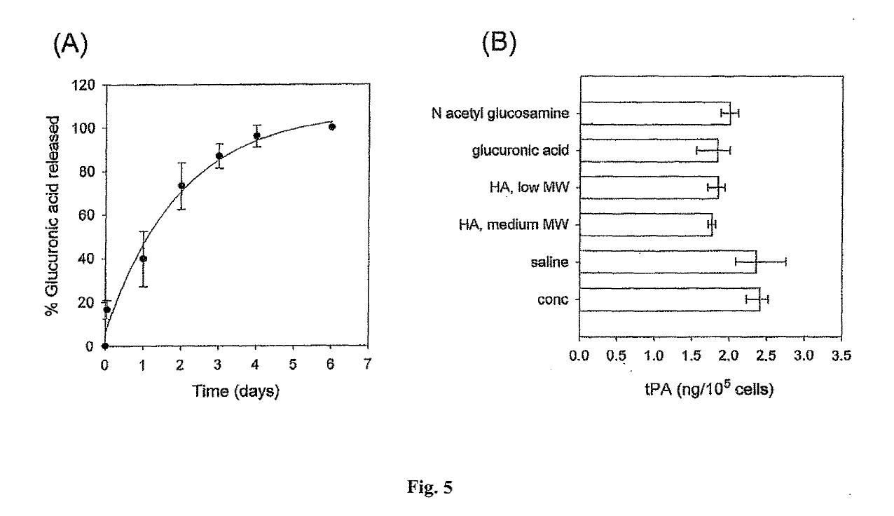

The balance between fibrinolysis and antifibrinolytic activity has been shown to be important in mediating the development of adhesions (Falk K, Bjorquist P, Stromqvist M, Holmdahl L. Reduction of experimental adhesion formation by inhibition of plasminogen activator inhibitor type 1. British Journal of Surgery 2001;88:286-289; Binda M M, Molinas C R, Koninckx P R. Reactive oxygen species and adhesion formation: clinical implications in adhesion prevention. Human Reproduction 2003; 18(12):2503-2507, each of which is incorporated herein by reference). Serosal fibrinolysis is mainly regulated by mesothelial release of t-PA and PAIs (Tietze L, Eibrecht A, Schauerte C, Klosterhalfen B, Amo-Takyi B, Gehlen J, et al. Modulation of pro- and antifibrinolytic properties of human peritoneal mesothelial cells by transforming growth factor beta1 (TGF-beta1), tumor necrosis factor alpha (TNF-alpha) and interleukin 1beta (IL-1beta). Thromb Haemost 1998;79(2):362-370, which is incorporated herein by reference). We investigated whether degradation products of HAX caused changes in mesothelial production of tPA and PAI-1, which might account for the reduction in adhesion formation from HAX gels. Cross-linked HA gels incubated in 10 U/ml hyaluronidase at 37° C. provided a steady release of such degradation products (FIG. 5A). Mesothelial cells were incubated in 1 ml medium with or without supplementation with 100 μl saline, or 100 μl of saline containing 20 mg/ml of HA (490 kD or 50 kD MW), or one of the two monomer components of HA (D-glucuronic acid or N-acetyl-D-glucosamine). FIG. 5B shows a statistically significant but small decrease in tPA production in mesothelial cells grown with hyaluronic acids of different Mw and the monomers as compared to untreated or saline treated controls (p=0.15). Difference among the groups treated with HA or monomers was not statistically significant (p=0.112). Similarly, PAI-1 production was not significantly affected by any treatment (data not shown). Without wishing to be bound by any theory, we believe it is likely that the significant reduction in adhesion formation with HAX gels was in large part due to the barrier function of the HAX gels. However, since the etiology of adhesion formation is mediated by a broad range of biological events, the fact that potential soluble leach-outs of the HAX gels (soluble HA, monomers) did not increase tPA production or affect PAI-1 levels in vitro cannot exclude the possibility of a biological effect as well.

### Example 5

### Effect of Monomer Concentration and Molecular Weight on HAX Gel Degradation

Materials and Methods

Degradation of HAX gels in hyaluronidase: HAX gels consisting of various concentrations of HA-ADH and HA-CHO having a range of molecular weights were prepared, and degradation of the gels in hyaluronidase was monitored over time. HAX gels were prepared by instantly mixing 150 μl of HA-ADH and 150 μl of HA-CHO of varying concentrations and Mw in 2 ml microcentrifuge tubes using a vortex mixer and then subjected to 37° C. incubation in hyaluronidase (50 U/ml in PBS). At predetermined time points, the hyaluronidase buffer was completely removed, and the wet mass of the remaining HAX gels was gravimetrically determined. The results were plotted as % gel mass at each time/original wet gel mass vs. time.

Results

To assess the potential for further optimization and control of the properties of HAX gels by changing the cross-linking density of the matrix, we studied the effect of varying concentration of the gel. The HAs used above were very viscous, and it was difficult to dissolve HAs above 20 mg/ml. Therefore, in order to increase the concentration of HA, we prepared lower-Mw precursors. To do this, we prepared HA-ADH from a 50 kD HA (instead of 490 kD), and HA-CHO from a 490 kD HA (instead of 1.36 MD). This allowed the formulation of 75 mg/ml HA-ADH and 60 mg/ml HA-CHO. In order to accelerate the gel degradation process and observe macroscopic changes of gel mass in reasonable time periods, degradation experiments were performed using 50 U/ml hyaluronidase.

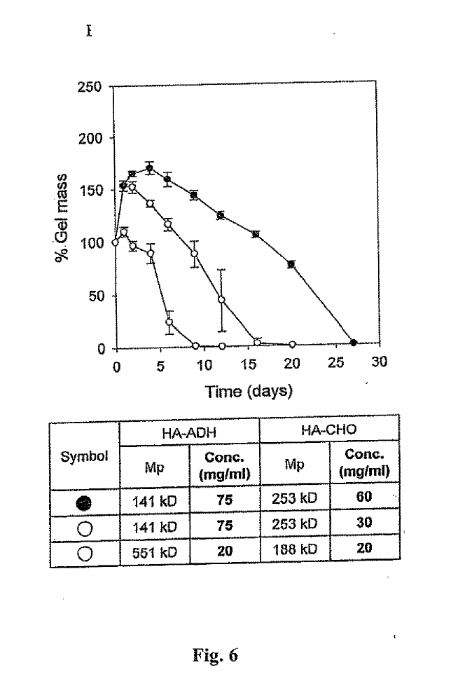

HAX gels of all concentrations swelled initially and then degraded at rates that depended on concentration (FIG. 6A) and molecular weight (FIG. 6B). The time for the hydrogel wet mass to decrease by 50% (‘half-life’) increased from 5 days to 11 days when the concentration of HA-ADH and HA-CHO solutions were increased from 20 mg/ml to 75 mg/ml and 30 mg/ml, respectively, and to 22.5 days when the concentrations were increased to 75 mg/ml and 60 mg/ml (FIG. 6A). (Note that in the latter comparison, the concentration of the HA-CHO was the only variable.) When the concentrations of HA-ADH and HA-CHO were kept constant, the half life was longer for the gel made with HA-CHO derived from higher Mw (>50 days for 1.36 MD HA-CHO vs. 22 days for 490 kD HA-CHO; FIG. 6B). In these experiments the HAX gels were cast in microcentrifuge tubes, where only one side of the gel faced the hyaluronidase solution. Therefore, the absolute half-lives shown here may not relate directly to the other experiments described here, where the gels were exposed to the enzyme solution on all surfaces.

The results presented in this example demonstrate that once formed, the HAX gel presents a durable physical barrier that may last for days to weeks (depending on the concentration and molecular weight of the gel components, see FIG. 6), until eventually degraded, e.g., by endogenous hyaluronidase. The fact that the time course of degradation can be controllably modulated by varying these parameters according to our approach allows this system to be tuned for particular applications, depending on the length of time for which the barrier function is desired.

### Example 6

### Preparation and Characterization of Hybrid HA/Nanoparticle Hydrogels

Materials and Methods

Materials. Hyaluronic acids (HA, 1.36 MDa and 490 kDa) were purchased from Genzyme Corporation (Cambridge, Mass.). Poly (lactic-co-glycolic) acid (PLGA, lactide:glycolide=65:35, Mw 90,000) was obtained from Alkermes (Cambridge, Mass.). Polyvinyl alcohol (PVA, Mw 6000) was purchased from Polysciences, Inc. (Warrington, Pa.). All other reagents were purchased from Sigma-Aldrich (St. Louis, Mo., USA) unless specified otherwise.

Preparation of cross-linkable hyaluronic acids and PLGA nanoparticles. In situ cross-linkable HA derivatives were synthesized as described in Example 1. Blank PLGA nanoparticles were prepared by the single emulsion method. PLGA 200 mg was dissolved in 5 ml of 3:2 mixture of methylene chloride and dimethylsulfoxide (DMSO). The polymer solution was directly added into 20 ml of 5% PVA. The mixture was then homogenized for 1 min using a sonicator (Vibracell VC-250, Sonics & Materials Inc., Dunbury, Conn.) to generate an oil-water emulsion. The formed emulsion was added into 100 ml distilled water and stirred overnight at room temperature. The remaining solvents were removed under reduced pressure. The nanoparticles were collected by centrifugation at 25,000 rpm for 20 min using an L8-70M ultracentrifuge (Beckman, Fullerton, Calif.) and an SW 28 swinging bucket rotor, and further purified by passing through a ultrafiltration membrane (Ultracel Amicon YM 100, Millipore, Billerica, Mass.) prior to lyophilization. Particle size was measured with a ZetaPALS zeta potential analyzer (Brookhaven Instruments Corporation, Holtsville, N.Y.).

Preparation of hydrogels. Cross-linked hyaluronic acid hydrogels without nanoparticles (“HAX”) were prepared by mixing 20 mg/ml solutions of gel precursors (HA-ADH and HA-CHO). Composite HAX gels containing PLGA nanoparticles (“hybrid”) were prepared by mixing 20 mg/ml solutions of gel precursors in 20 mg/ml PLGA nanoparticle suspension.

Scanning electron microscopy. The surface morphology of PLGA nanoparticles and the internal structure of lyophilized hydrogels were examined by scanning electron microscopy. The hydrogels were made by mixing equal volume of precursor solutions prepared in distilled water. The hydrogels were then lyophilized and fractured after cooling in liquid nitrogen. The samples were sputter-coated with palladium and gold (100 Å thick) and observed using a scanning electron microscope (JEOL JSM 6320, JEOL USA, Inc., Peabody, Mass.).

Gelation time. A hundred μl of HA-ADH solution containing PLGA nanoparticles (both HA-ADH and PLGA nanoparticles were either 20 mg/ml or 10 mg/ml in saline) was added into an 8×35 mm glass vial, which contained a magnetic stirring bar (Teflon fluorocarbon resin, 5×2 mm, Fisher Scientific). A hundred μl of HA-CHO solution containing PLGA nanoparticles (both HA-CHO and PLGA nanoparticles were either 20 mg/ml or 10 mg/ml in saline) was then added to the vial, and the solution was stirred at 155 rpm using a Corning model PC-320 hot plate/stirrer until hybrid gel was formed. The gelation time was considered to be the time when the solution formed a solid globule, which completely separated from the bottom of the dish. For comparison, formation of HAX gel was also tested. The results are reported as averages and standard deviations of 4 independent measurements.

Rheological testing. Cylindrical HAX and hybrid gels were prepared by adding 20 mg/ml gel precursor solutions using 1-ml syringes and a Baxter dual valve applicator into a rubber mold sandwiched between two slide glasses. The diameter and the thickness of the prepared hydrogel were 8 mm and 3.5 mm, respectively. Gels were then transferred to an AR1000N rheometer (TA Instruments, New Castle, Del.) for rheological measurements. All experiments were conducted using a parallel 8-mm diameter plate at room temperature. Shear modulus, G, was measured by the creep test and the stress sweep test. For the creep test, the hydrogels were subjected to a constant shear stress (5, 10, 20, or 40 Pa) for 90 seconds and then allowed to recover for 90 seconds. After 60 seconds in each creep and recovery step, the strain reached a constant value. G was determined as a reciprocal of the slope of the strain (read at the end of the recovery step) versus stress curve. For the stress sweep test, oscillatory stress was applied in the range 1-100 Pa at a constant frequency (0.1 Hz). Elastic modulus, G′, obtained at 40 Pa was used as an approximation of G because viscous modulus, G,″ was close to 0. The results are reported as averages and standard deviations of 4 independent measurements.

Statistical analysis for Examples 6-8. Rheological measurements are reported as means and standard deviations, and are compared using the Student t-test. Cell culture data were expressed as medians with 25^{th} and 75^{th} percentiles since they did not always follow a normal distribution; scores were also reported this way. For these, statistical inferences were made using Mann-Whitney U-tests, Kruskal-Wallis tests, or Fisher's exact test, using SPSS software (Chicago, Ill.). A p-value<0.05 on a 2-tailed test was considered statistically significant.

Results

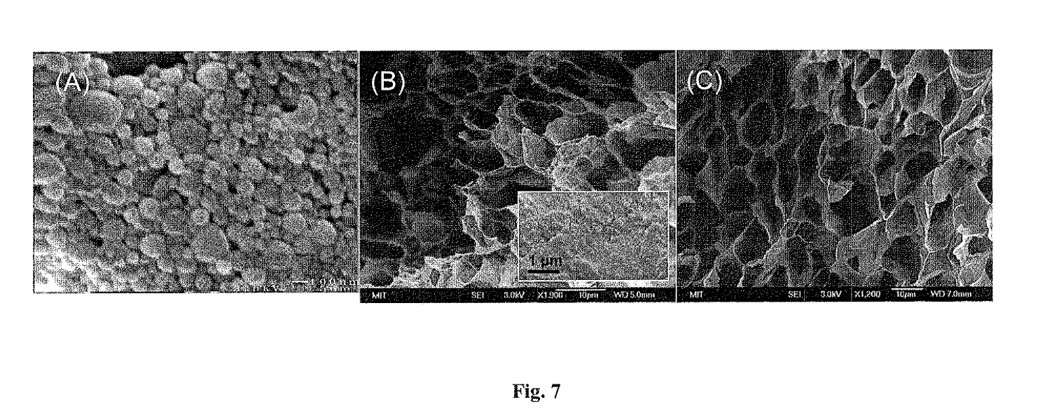

Characterization of hybrid gels. The average diameter and zeta potential of PLGA nanoparticles were 278.4±18.7 nm and −18.1±4.0 mV, respectively. To scanning electron microscopy, the particle size distribution was relatively broad ranging from 50 nm to 300 nm (FIG. 7A). Lyophilized hybrid gel matrices had continuous pores with a diameter ranging from 5 to 10 μm (FIG. 7B) that were comparable to those of HAX gels (FIG. 7C). The hybrid gels had rough surface (FIG. 7B inset) indicating the nanoparticles embedded in the gel matrix. As described herein, one of the advantages of HAX gels as adhesion barriers is that they can cross-link in situ within a suitable time frame for treatment of adhesions. To assess whether incorporation of polymeric nanoparticles interfered with gelation, we compared the gelation times of HAX and hybrid gels. Both systems gelled rapidly upon mixing, without statistically or practically significant difference between the two (Table 3). Similarly, we wished to ascertain whether nanoparticles affected the mechanical properties of the gels. The shear modulus was unaffected by nanoparticles (Table 3).

TABLE 3
Comparison of gelation time and shear modulus
of HAX and hybrid gels

| | | Gel conc. | HAX | Hybrid |
|---|---|---|---|---|
| Gelation | | 20 mg/ml | 3.5 ± 0.6 | 4.5 ± 0.6 |
| time (sec) | | 10 mg/ml | 4.8 ± 0.5 | 6.0 ± 1.4 |
| Stress | Creep test* | 20 mg/ml | 243.6 ± 38.4 | 332.2 ± 81.2 |
| modulus, G | Stress sweep | | 327.9 ± 62.3 | 419.4 ± 104.7 |
| (Pa) | test* | | | |

### Example 7

### Biocompatibility of Hybrid HA/Nanoparticle Hydrogels

Materials and Methods

Hydrogels. Hybrid HA/nanoparticle hydrogels were prepared as described in Example 6.

Cell proliferation assay. Human mesothelial cells (ATCC, CRL-9444) were cultured in Medium 199, containing Earle's salts, L-glutamine, and 2.2 g/L sodium bicarbonate and supplemented with 3.3 nM epidermal growth factor, 400 nM hydrocortisone, 870 nM insulin, 20 mM HEPES, and 10% fetal bovine serum (Invitrogen). Cells were seeded into 24-well plates at a density of 50,000 cells per well in 1 ml of culture media. After overnight, 100 μl cylindrical hybrid gels or saline were added to each well. After varying periods of incubation in the presence of the hydrogels, cell viability was assessed with an MTT assay kit (Promega CellTiter 96 Non-Radioactive Cell Proliferation Assay). Results were reported as medians with 25^{th} and 75^{th} percentiles of the measured absorbance normalized to the absorbance of non-treated control cells (% normalized cell viability=100×Absorbance for cells grown in the presence of a sample in medium/absorbance for cells grown in medium).

Results

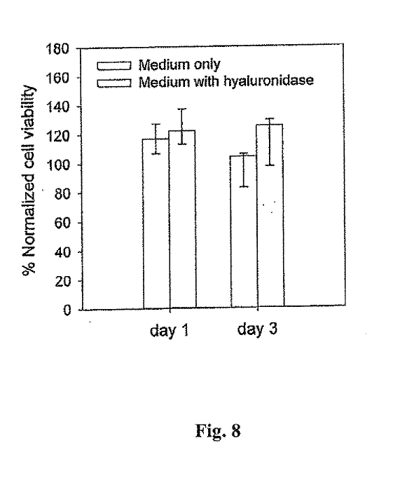

The effect of hybrid gels on in vitro mesothelial cell viability was assessed with the MTT assay. Cells were grown in the presence of cylindrical 100 μl hybrid gels (20 mg/ml HAX+20 mg/ml PLGA nanoparticles) measuring 3.5 mm×8 mm diameter, for up to 3 days. This was done in medium with or without 10 units/ml hyaluronidase, an enzyme that degrades HAX. (In the controls, that provided the denominator for normalizing viability, 100 μl of normal saline was added instead of the gel) Cell viability was well maintained at 1 and 3 days of incubation, irrespective of the presence of hyaluronidase. There were no statistically significant differences between the groups shown in FIG. 8.

### Example 8

### Prevention of Adhesions in a Mouse Model by In Situ Cross-Linked Hybrid HA/Nanoparticle Gel

Materials and Methods

Hydrogels. Hybrid HA/nanoparticle hydrogels were prepared as described in Example 6.

In vivo application of hybrid gel. Animals were cared for in compliance with protocols approved by the Massachusetts Institute of Technology Committee on Animal Care, in conformity with the NIH guidelines for the care and use of laboratory animals (NIH publication #85-23, revised 1985).

Mouse intraperitoneal injection model. Male SV129 mice weighing 20-35 g were used. One ml of sterile hybrid gel was injected into the peritoneum via a single puncture in the left lower quadrant. Prior to application, the materials were sterilized by germicidal UV illumination for 2 hours and dissolved in sterile saline. HA-ADH with PLGA nanoparticles 0.5 ml (10 mg/ml HA-ADH and 20 mg/ml nanoparticles) and HA-CHO 0.5 ml (10 mg/ml containing 20 mg/ml nanoparticles) were placed in separate sterile 1-ml syringes, which were connected to a Baxter dual valve applicator, and co-extruded through a 20-gauge needle. Animals were euthanized with carbon dioxide 2 or 7 days after injection. Animals were examined as to whether adhesions were present or not, and whether hybrid gel residue was visible or not.

Histological examination. Tissues recovered from the necropsy were fixed in 10% formalin, embedded in paraffin, sectioned, and stained with hematoxylin and eosin (H&E) using standard techniques.

Results

Development of a new approach to peritoneal drug delivery. There is considerable interest in the development of methods to deliver drugs to the peritoneum for the prevention and/or treatment of peritoneal adhesions and for other purposes. However, drug delivery to the peritoneum is hampered by rapid clearance. We sought to determine whether peritoneal drug delivery could be improved by application of polymer-based controlled release technology. We investigated the suitability for peritoneal use of micro- and nanoparticles of poly(lactic-co-glycolic) acid (PLGA), a biodegradable polymer with generally excellent biocompatibility commonly used for controlled drug release. We injected 90 kDa PLGA microparticles, 5 to 250 μm in diameter, into the murine peritoneum, in dosages of 10 to 100 mg (n=3-5 per group). We found a high incidence of polymeric residue and adhesions 2 weeks after injection (e.g. 50 mg of 5 μm microparticles caused adhesions in 83% of animals). Histology revealed chronic inflammation, with foreign body giant cells prominent with particles >5 um in diameter. Five μm microspheres made from 54, 57, and 10 kDa PLGA (gamma irradiated) caused fewer adhesions (16.7%) with a similar incidence of residue. Nanoparticles (265 nm) of 90 kDa PLGA also caused far fewer adhesions (6.3% of animals), possibly because they were cleared from the peritoneum within 2 days and sequestered in the spleen and liver, where foamy macrophages were noted. These experiments suggested that neither the microparticles nor nanoparticles that we tested would alone provide an acceptable polymeric drug delivery system for use in the peritoneum. We hypothesized that a composite hydrogel system for intraperitoneal drug delivery in which particles are entrapped within an in situ cross-linkable hyaluronic acid hydrogel would act as a barrier to inhibit formation of adhesions while allowing the effective use of polymer-based drug delivery. In particular, we hypothesized that in the case of particles that might otherwise contribute to adhesion formation, the hydrogel would at least in part prevent the enclosed particles from contributing to adhesion formation. In the case of particles that would otherwise be rapidly cleared from the peritoneum, the hydrogel would retain the particles within the peritoneum.

Biocompatibility in the mouse intraperitoneal injection model. To test the hypothesis that the HAX would keep nanoparticles within the peritoneum and at the same time prevent the formation of adhesions from the presence of high molecular-weight PLGA, mice peritoneums were injected with 1 ml of 10 or 20 mg/ml HAX containing 20 mg of PLGA nanoparticles. Animals injected with nanoparticles in the absence of HAX had previously been shown to leave the peritoneum within 2 days, leaving little polymeric residue, and to frequently have enlarged, discolored spleens with foamy macrophages (data not shown).

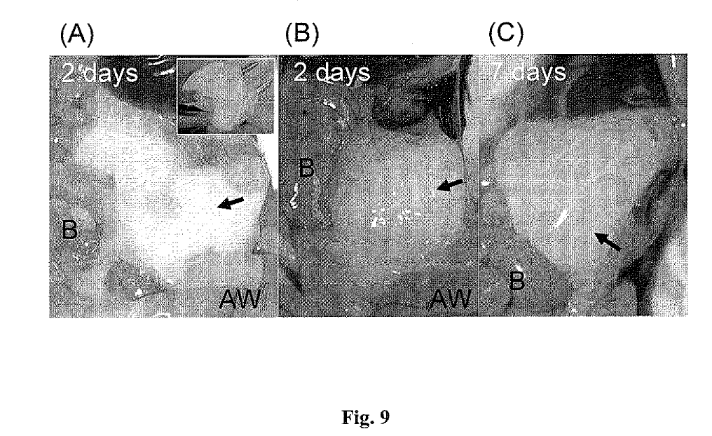

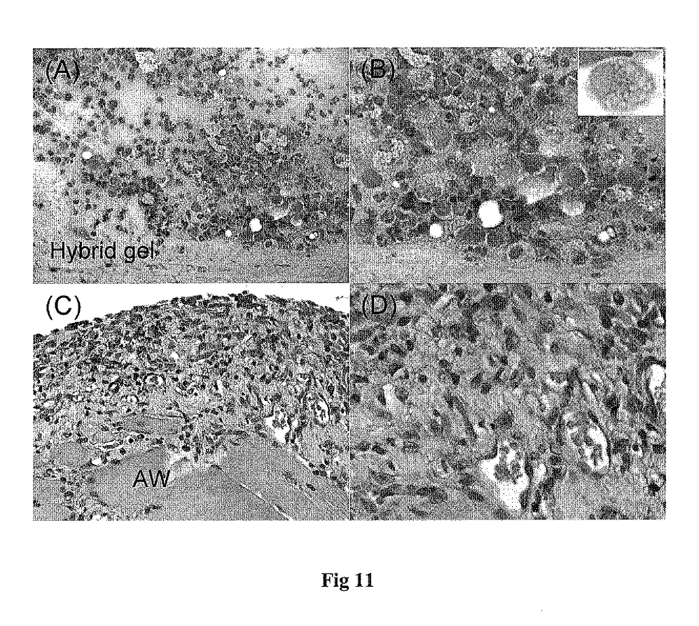

On necropsy 2 days after injection (Table 4), the hybrid gels containing HAX 10 mg/ml remained at the injection site as discrete masses that were easily separated from the surrounding abdominal contents (FIG. 9A). The hybrid gels containing HAX 20 mg/ml seemed more adherent to the abdominal contents, but were still easily separable from the contacting organs, although they did leave some residue (FIG. 9B). No adhesions or abnormalities in spleen size or color were noted in either group. A further hybrid group containing 20 mg/ml HAX was sacrificed 7 days after injection. Gel was seen in 3 out of 4 mice (FIG. 9C), with gel masses were comparable to those recovered after 2 days. In the single case of adhesion seen in this group, there was very little or no gel visible, suggesting that the bowel had been penetrated, with the possible intraluminal injection of gel (and presumable subsequent clearance in the stool). Here also, the spleens seemed grossly normal. Foamy macrophages were noted on light microscopy of stained slides of the gels recovered from the abdominal cavities of many animals, suggesting the presence of retained nanoparticles (FIG. 11B, insert). Foamy macrophages were not seen in the liver or spleen. Increased vascularity was noted at the periphery of many gels, as can be seen in all panels of FIG. 9.

TABLE 4
Biocompatibility of hybrid gel in the mouse model

| | | | |
|---|---|---|---|
| HAX (mg) | 10 | 20 | 20 |
| PLGA nanoparticle (mg) | 20 | 20 | 20 |
| Days to dissection | 2 | 2 | 7 |
| n | 4 | 4 | 4 |
| Adhesion | 0 | 0 | 1 |
| Adhesion % | 0 | 0 | 25 |
| Presence of residual gel | 4 | 4 | 3 |

### Example 9

### Prevention of Peritoneal Adhesions in a Rabbit Abrasion Model by in Situ Cross-Linked Hybrid HA/Nanoparticle Gel

Materials and Methods

Hydrogels. Hybrid HA/nanoparticle hydrogels were prepared as described in Example 6.

In vivo application of hybrid gel. Animals were cared for in compliance with protocols approved by the Massachusetts Institute of Technology Committee on Animal Care, in conformity with the NIH guidelines for the care and use of laboratory animals (NIH publication #85-23, revised 1985).

Rabbit sidewall defect-cecum abrasion model. Female albino rabbits (Oryctolagus cuniculus; New Zealand White, Covance, Hazleton, Pa.) (3±0.5 kg) were used as model animals. Post-surgical adhesions were induced as described in Example 3. Briefly, after the peritoneum was opened by a 10 cm long midline incision along the linea alba on the abdominal wall, a 3×4 cm defect comprising the parietal peritoneum and a layer of muscle (˜1 mm thick) was made on the right lateral abdominal wall starting 1 cm from the midline. Subsequently, the anti-mesenteric side of the cecum was abraded biirectionally for 80-160 times from the 6^{th} haustra distal to the ileocecal junction to the 12^{th} haustra using a sterile surgical brush resulting in bleeding.

Twenty animals were assigned randomly to experimental groups: (i) no treatment (n=12); (ii) covering the excised abdominal wall and cecal surface with 10 ml of sterile hybrid gels. Five ml of HA-ADH with PLGA nanoparticles (20 mg/ml HA-ADH and 20 mg/ml nanoparticles) and 5 ml of HA-CHO (20 mg/ml HA-CHO and 20 mg/ml nanoparticles) were placed in separate sterile 10-ml syringes, which were connected to a Baxter dual valve applicator, and co-extruded through a 15-gauge needle. The liquid precursors started to gel instantly, conforming to the shape of the applied area. To visual exam, gelation was complete in less than 3 minutes: the hydrogel did not flow beyond that point.

Post-operative care was taken as described in Example 3. One week after the procedure, animals were euthanized with sodium pentobarbital. Adhesions were scored following a reported method (Burns J W, Skinner K, Colt J, Sheidlin A, Bronson R, Yaacobi Y, et al. Prevention of tissue injury and postsurgical adhesions by precoating tissues with hyaluronic acid solutions. Journal of Surgical Research 1995;59:644-652, which is incorporated herein by reference) with modification. Score-0 represents no adhesion, score-1 tissue adherence separated with gravity, score-2 tissue adherence separable by blunt dissection, and score-3 adhesion requiring sharp dissection. The location of adhesions (whether they include suture sites or not; location and number of cecal haustra involved in the adhesion) and weight changes were also recorded.

Histological examination. Tissues recovered from the necropsy were fixed in 10% formalin, embedded in paraffin, sectioned, and stained with hematoxylin and eosin (H&E) using standard techniques.

Results

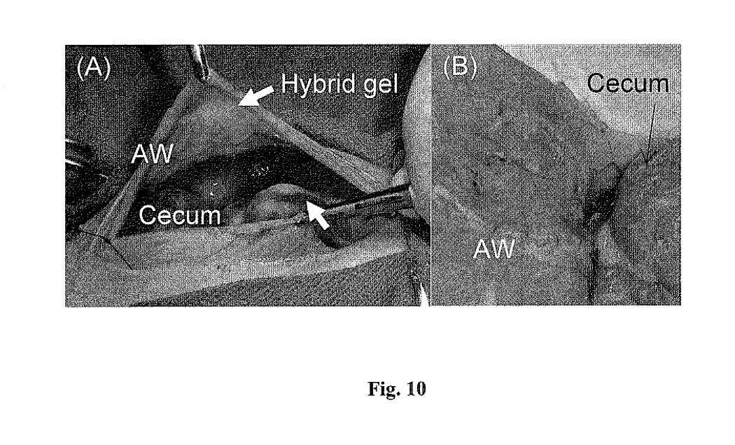

Biocompatibility and effectiveness of hybrid gels in an abdominal sidewall defect-cecum abrasion model in the rabbit. Eight rabbits received laparotomies in which the cecum was abraded and a segment of the adjacent abdominal wall was excised, as described in Methods. The hybrid gel (20 mg of nanoparticles in 10 ml of 20 mg/ml HAX) was painted on the injured sites. At the time of necropsy one week later (Table 5), their weight loss was comparable to that in a control group (same injury, no treatment, n=12). The median adhesion score was dramatically lower in the treated group (p=0.002, Mann-Whitney U-test; 0.001, Fisher's exact test). While 83.3% of animals in the control group developed score 3 adhesions (firm links that could only be separated by cutting) (Example 3), none in the treated group developed score 3 adhesions (p<0.001, Mann-Whitney U-test; p=0.001, Fisher's exact test). Five out of 8 animals (62.5%) in the treated group showed no tissue adherence at all (FIG. 10B), compared to 2 of 12 (16.7%) in the control group (0.04, Mann-Whitney U-test; p=0.035, Fisher's exact test).

Of the three animals that developed score 2 adhesions, one was between the cecal surface and the excised abdominal wall near the suture site, one between abraded cecum and non-abraded cecum, and one developed two score 2 adhesions between abraded cecum and non-abraded cecum and between the cecal surface and the injured abdominal wall near the suture site. Therefore, many of the sites where these score 2 adhesions occurred were sites that were not covered with the hybrid gel. On necropsy, the hybrid gels were noted to still be at the sites where they were applied, but the quantity and mechanical properties were significantly reduced. In some cases, the cecal surfaces covered by the remnants of the hybrid gels retained traces of old blood from the original abrasion. On light microscopy, foamy macrophages, presumed to contain polymeric debris, were noted in stained slides of gel residue (FIGS. 11A and B); free polymer (bright spots) was also noted. Foamy macrophages were not found in liver or spleen (not shown). They were also found on the surface of adhesion-free injured abdominal wall (FIGS. 11C and D), where hybrid gel had been applied.

TABLE 5
Evaluation of peritoneal adhesions in the rabbit model

| | No treatment (n = 12) | | | |
|---|---|---|---|---|
| | (see Example 3) | | Hybrid (n = 8) | |
| % weight change | −5.7 ± 4.6 | | −5.5 ± 3.6 | |
| Adhesions | Frequency | Percentage | Frequency | Percentage |
| Score 3 | 10 | 83.3 | 0 | 0 |
| Score 2 | 0 | 0 | 3 | 37.5 |
| Score 1 | 0 | 0 | 0 | 0 |
| No adhesion | 2 | 16.7 | 5 | 62.5 |

| | | |
|---|---|---|
| Adhesion score* | 3 (3; 3) | 0 (0; 0.5) |

Median with 25^{th} and 75^{th} percentiles in parentheses.

Our data show that the hybrid system has low cytotoxicity in vitro to peritoneal mesothelial cells, is biocompatible in the peritoneum in vivo, and is intrinsically capable of preventing adhesions. With respect to the latter, it performs at least as well as HAX (Example 3). Incorporation of nanoparticles into the HAX had essentially no effect on the gelation time or shear modulus of the gel system. The HAX successfully maintained the nanoparticles within the peritoneum for the duration of the experiment, as seen by the presence of foamy macrophages in the gel remnants and the lack of such cells in liver and spleen—where they had been noted in mice injected with comparable masses of nanoparticles without HAX. HAX also prevented the formation of adhesions from the retained polymer.

This gel system, which forms in situ, is easy to use with a double-barreled syringe or similar device. The relatively rapid gelation time allows the user to apply gel to specific locales without spillage into adjacent regions. As discussed above, gelation time can be modified by changing polymer concentration and/or molecular weight or crosslinking density. This system can therefore be easily applied by a laparoscope or by percutaneous injection. Potential uses are not restricted to the peritoneum and can be used to administer a wide variety of therapeutic agents including agents that inhibit formation of adhesions and/or agents that have other desirable effects.

The hybrid gels were highly efficacious in preventing peritoneal adhesions in a rabbit sidewall defect-cecum abrasion model. This was particularly clear when comparing the prevalence of score 3 adhesions, which were firm links that could only be separated by sharp dissection. There were no score 3 adhesions in animals treated with the hybrid gel compared to an incidence of 83.3% in untreated animals (Example 3). Similarly, 62.5% of treated animals showed no adhesions compared to 17% in controls. The three cases of score-2 tissue adherence (separable by blunt dissection) occurred either near the suture site or between two cecal surfaces (one of which was not abraded), i.e. all adhesions involved areas which were not covered by the hybrid gel. Here, the gel was applied within the confines of the sites of injury. Application of the system across a larger surface area may further increase effectiveness. In conclusion, the hybrid HAX-nanoparticle system described here appears to be a suitable intraperitoneal drug delivery system as well as an effective barrier for preventing post-surgical adhesions.

### Example 10

### Prevention of Peritoneal Adhesions in a Repeated Laparotomy Model by In Situ Crosslinked HA Gel Containing tPA

Materials and Methods

Optimization of gel concentration. HA-ADH and HA-CHO were prepared as described above. HAX gels were prepared with solutions of varying Mw of HA-ADH and HA-CHO at different concentrations, including the maximum attainable concentrations. HAX gels were prepared by mixing 150 μl of HA-ADH and 150 μl of HA-CHO solutions in 2 ml microcentrifuge tubes using a vortex mixer and then incubating at 37° C. in hyaluronidase (50 U/ml in PBS). At predetermined time points, the hyaluronidase buffer was completely removed, and the wet mass of the remaining HAX gels was determined gravimetrically.

Repeated laparotomy model. Female albino rabbits (Oryctolagus cuniculus; New Zealand White) (3±0.5 kg) were used. The first laparotomy and post-operation care were performed as described above. A 2^{nd} laparotomy was performed after 1 week. On examination prior to the 2^{nd} laparotomy, animals were excluded from the 2^{nd} laparotomy for the following: (i) loss of more than 15% of body weight since the 1^{st} laparotomy; (ii) poor feeding. On laparotomy, adhesions were scored as previously then lysed (cut). The previously excised abdominal wall was re-abraded 50 times unidirectionally, and the cecum surface between 6-12^{th} haustra was re-abraded 150-200 times bidirectionally using a sterile brush until a bleeding bed was obtained.

Thirty animals were assigned randomly to experimental groups and test materials were applied on the injured areas. The operator was blinded as to the nature of the materials. HA-ADH and HA-CHO were sterilized by germicidal UV illumination for 2 hours and dissolved in 5 ml sterile saline, respectively. Ten ml of gel were applied on the injured sites with a dual valve applicator as previously. As controls, a group of animals received no treatment, and another group received 10 ml saline.

After treatment of the injured areas, the peritoneum and abdominal wall were closed with 2-0 Ethilon and 3-0 Dexon, respectively. The skin was closed with 2-0 Ethilon. Animals were allowed to awaken and had food and water ad libitum. Buprenorphine 0.02-0.03 mg/kg was administered sc 8-12 hours interval until 48 hours post-surgery. The animals were sacrificed 1 week after the second laparotomy by intravenous injection of sodium pentobarbital. Adhesions were scored in two ways. (i) Quality of adhesions was scored as follows: Adhesions were scored using a modification of a reported method (Burns J W, Skinner K, Colt J, Sheidlin A, Bronson R, Yaacobi Y, et al. Prevention of tissue injury and postsurgical adhesions by precoating tissues with hyaluronic acid solutions. Journal of Surgical Research 1995;59:644-652, which is incorporated herein by reference). Score 0=no adhesion, score 1=tissue adherence that would separate with gravity, score 2=tissue adherence separable by blunt dissection, score 3=adhesion requiring sharp dissection. If there were multiple adhesions of different scores, we chose the higher one as a representative score. (ii) Area of score 2 or 3 adhesions was measured for quantitative evaluation of the adhesions. The location of adhesions (whether they include suture sites or not; location and number of cecal haustra involved in the adhesion) and weight changes were recorded. Tissues recovered from the necropsy were fixed in 10% formalin, embedded in paraffin, sectioned, and stained with hematoxylin and eosin for histological examination.

Results

Increasing the concentration of the previously used HA solutions was impeded by their viscosity (in particular HA-ADH). To further increase the concentration, we prepared modified HAs using lower Mw HAs. As shown in Table 6, the maximum practical concentration (i.e. that could be manipulated with pipettes or syringes) increased with decreasing Mw of HAs.

TABLE 6
Maximum concentration of modified HA solutions

| HA-ADH* | Concₘₐₓ (mg/ml) | HA-CHO* | Concₘₐₓ (mg/ml) |
|---|---|---|---|
| 10 kD | 180 | 50 kD | 150 |
| 50 kD | 75 | 490 kD | 60 |
| 490 kD | 20 | 1360 kD | 60 |

*Note that the listed Mw are nominal Mw of original HAs.

Optimization of gel concentration. All HAX gels with increased concentrations lasted significantly longer than 20 mg/ml HAX gel (Table 7). Note that it was not the highest concentration gel that lasted longest. Despite the high density, a gel consisting of low Mw HAs (i.e. having many nicks on the backbones) appeared to have undergone fast mass loss in the later stage of degradation. Among the tested HA combinations, 75 mg/ml HA-A (50 kD) and 60 mg/ml HA-B (1.36 MD) provided the most slowly degrading HAX gel. (t_{1/2} in 50 u/ml HAse: 51 days). The gel formed from 75 mg/ml HA-ADH (50 kD)+60 mg/ml HA-CHO (1.36 MD) is referred to as HAXₕₓ.

TABLE 7
Half-life of HAX gel in 50 u/ml HAse

| | | T_{1/2} (days)^{a} in 50 u/ml |
|---|---|---|
| HA-ADH | HA-CHO | HAse |
| 490k, 20 mg/ml | 1.36M, 20 mg/ml | 5 |
| 490k, 20 mg/ml | 50k, 150 mg/ml | 11 |
| 50k, 75 mg/ml | 50k, 150 mg/ml | 27 |
| 10k, 180 mg/ml | 50k, 150 mg/ml | 17 |
| 50k, 75 mg/ml | 1.36M, 60 mg/ml | 51^{b} |
| 50k, 75 mg/ml | 490k, 60 mg/ml | 22.5 |

^{a}The time for the hydrogel wet mass to decrease by 50%
^{b}Determined by extrapolation.

Development of repeated laparotomy model. This model produces extensive, reproducible adhesions. It is not commonly used, because the exuberant adhesions it creates are extremely challenging to treat. The repeated laparotomy model allows us to evaluate the effectiveness of our candidate materials in preventing severe adhesions and/or recurrent adhesions, on which commercial products have only shown limited effects. Furthermore, this model allows us to identify materials that are even more effective than those described in the preceding examples.

Out of 30 animals, 29 developed score-3 adhesions (also partially score 2) after the first laparotomy (96.7%). The adhesions mostly involved the excised abdominal wall and abraded cecum haustra, but often involved non-abraded cecum (mostly around ileocecal junction, because its location makes it contact the excised abdominal wall). The animals lost −6.1±3.9% of body weight during the first week after the first laparotomy. The adhesions were carefully lysed by sharp or blunt dissection. The lysis often resulted in deep injury on the abdominal wall and subsequent bleeding. The re-abraded cecum bled more easily than in the initial abrasion. When the incision was closed without any treatment, 100% (n=6) had developed score 3 adhesions when re-explored 1 week later. The adhesions were not limited to the injured locations, but involved the suture line on the abdominal wall, uninjured cecal surfaces, and/or intercecal, inter cecum-proximal colon surfaces. The median total area of adhesions was 12.7 (25%: 9.4, 75%: 16.6) cm². During the 1 week survival period following the second laparotomy, the animals lost −3.5±7.4% of body weight.

In vivo effects of hydrogels. The in vivo efficacy of tPA-HAXₕₓ gels was tested using the repeated laparotomy model and compared with that of HAX and with that of a hybrid gel containing PLGA nanoparticles in a 1:1 particle: HA derivative ratio (w/w). As additional controls, we also administered (i) tPA-HAXₕₓ gels in which the tPA had been inactivated by heat treatment; (ii) a bolus of tPA solution in the absence of HAX. The experimental protocol is summarized below:

[image]

The results are summarized in Table 8. Briefly, tPA-HAX with high crosslinking density (‘hx’), achieved by using a high concentration of HA derivatives was most effective in preventing adhesions in the double injury model. While HAX (hx) (no tPA) was not significantly effective, inactive tPA-HAX (hx) and bolus tPA (tPA solution was applied on the injured tissue) both contributed to reducing adhesion area. Without wishing to be bound by any theory, this effect may be related to the inactive ingredients in Activase (tPA, Genzyme). Activase containing 100 mg tPA also contains 3.5 g L-Arginine, 1 g phosphoric acid, Polysorbate 80 (<11 mg). Thus these agents are suitable for inclusion in a hydrogel of the present invention either individually or in combination.

In summary, HAX and hybrid gels could effect a relatively modest reduction in high-grade adhesions (approx. 17%) in this model, while HAX containing tissue plasminogen activator (tPA) dramatically reduced the incidence of high-grade adhesions by 60%, and reduced the surface area of those adhesions one hundred-fold. Importantly, this therapeutic benefit was obtained without incurring systemic bleeding, which has been a major problem reported with use of free tPA.

TABLE 8
Efficacy of HAX gels containing tPA.

| | | | | | | % Weight change | Qualitative (score) | | | | Quantitative | | |
|---|---|---|---|---|---|---|---|---|---|---|---|---|---|
| | | | | | | after 2^{nd} | % frequency | | | | (adhesion area, cm²) | | |
| Treatment | HA-ADH | HA-CHO | PLGAnp | tPA | n | laparotomy | 0 | 1 | 2 | 3 | median | 25% | 75% |
| No treatment | — | — | — | — | 6 | −3.5 ± 7.4 | 0 | 0 | 0 | 100 | 12.7 | 9.4 | 16.6 |
| Saline | — | — | — | — | 6 | −4.8 ± 1.8 | 0 | 0 | 0 | 100 | 15.4 | 11.1 | 17.8 |
| HAX | 100 mg | 100 mg | — | — | 6 | −3.0 ± 3.1 | 0 | 0 | 16.7 | 83.3 | 4.9 | 2.6 | 7.1 |
| Hybrid | 100 mg | 100 mg | 200 mg | — | 6 | −5.5 ± 2.0 | 16.7 | 0 | 0 | 83.3 | 12.2 | 1.6 | 19.1 |
| tPA¹-HAX(hx) | 375 mg | 300 mg | — | 2.2 mg | 9 | −4.4 ± 3.2 | 55.6 | 0 | 0 | 44.4 | 0.0 | 0.0 | 0.2 |
| HAX(hx) | 375 mg | 300 mg | — | — | 4 | −7.8 ± 1.5 | 0 | 0 | 0 | 100 | 11.1 | 9.8 | 11.7 |
| Inactive | 375 mg | 300 mg | — | 2.2 mg | 4 | −10.0 ± 2.5 | 0 | 0 | 0 | 100 | 1.5 | 0.3 | 4.6 |
| tPA²-HAX(hx) | | | | | | | | | | | | | |
| tPA | — | — | — | 2.2 mg | 4 | −3.4 ± 2.2 | 25 | 0 | 0 | 75 | 2.4 | 1.2 | 4.4 |

¹tPA was used as supplied by Genzyme (2.2 mg Activase + Larginine + phosphoric acid + polysorbate 80).
²tPA was inactivated by boiling for 20 min. Disruption of protein conformation was confirmed with ELISA.

### Example 11

### Prevention of Peritoneal Adhesions by Hydrogels Formed by In Situ Crosslinking of HA and Cellulose Derivatives

This example describes development of in situ crosslinkable hydrogels composed of HA and cellulose derivatives such as CMC (carboxymethylcellulose), MC (methyl cellulose), and HPMC (hydroxypropylmethyl cellulose).

Materials and Methods

Preparation of HA-ADH, HA-CHO, CMC—CHO, MC—CHO, HPMC—CHO: The protocol was essentially the same as that used for the synthesis of HA-CHO, which was described above.

Preparation of disk hydrogels: Aqueous solutions of 2 wt % HA-ADH and 2 wt % HA-CHO, CMC—CHO, HPMC—CHO, or MC—CHO were mixed in a rubber mold sandwiched between two slide glasses using a double syringe. The diameter and the thickness of the prepared hydrogels were 1.2 cm and 3.5 mm, respectively.

Results

Synthesis and characterization of CMC—CHO, HPMC—CHO, and MC—CHO. Successful synthesis was demonstrated by NMR and FT-IR. The weight-average molecular weights, M_{w}, measured by GPC were from 10³ to 10⁷. The modification degree of HA-CHO, CMC—CHO, HPMC—CHO, and MC—CHO were around 50%.

Gelation time of HA-CMC, HA-HPMC, and HA-MC: A variety of hydrogels were formed by crosslinking of HA-ADH and different aldehyde polysaccharides such as CMC—CHO, MC—CHO, and HPMC—CHO. The gelation times of HAX (formed by crosslinking HA-ADH and HA-CHO), HA-HPMC, HA-MC, and HA-CMC ranged from about 3-18 seconds, as shown in Table 9, demonstrating their suitability for in situ crosslinking.

Shear modulus, G, of HA-CMC, HA-HPMC, and HA-MC: The measured G values measured by rheometer were shown in Table 9. HA-HPMC and HA-MC showed relatively high values.

TABLE 9
Gelation time and Shear modulus of HA-CMC,
HA-HPMC, and HA-MC

| | Gelation time | G |
|---|---|---|
| | (sec) | (Pa) |
| HAX | 3.5 ± 1.0 | 32.4 ± 17.2 |
| HA-CMC | 18.5 ± 1.7 | 91.7 ± 19.3 |
| HA-HPMC | 4.0 ± 1.2 | 291.7 ± 108.6 |
| HA-MC | 5.8 ± 2.9 | 296.8 ± 40.7 |

Injection of hydrogels into peritoneum. 1 ml hydrogels, which consist of 0.5 ml 2 wt % HA-ADH and 0.5 ml 2 wt % CMC—CHO (or MC—CHO, HPMC—CHO), were injected into mouse peritoneum. These hydrogels were very biocompatible following injection into the peritoneum of mice. Four days after injection, HA-HPMC gels were found as cohesive masses. On the other hand, HA-CMC and HAX were spread throughout the cavity and covered all the organs. HA-MC had an intermediate structure/consistency. Two weeks after injection, there was almost no residue in animals injected with HAX. HA-HPMC gels still remained at 2 weeks. HA-CMC gels also persisted and covered the viscera as a thin layer. The injection of these gels did not cause peritoneal adhesions, as shown in Table 10.

TABLE 10
Prevalence of adhesions

| Hydrogel | 4^{th} day | 1st week | 2nd week | 3rd week | Total |
|---|---|---|---|---|---|
| HAX | 0/2 | 0/1 | 0/1 | 0/1 | 0/5 |
| HA-CMC | 0/3 | 0/1 | 0/1 | 0/6 | 0/6 |
| HA-HPMC | 0/2 | 0/1 | 1/1 | 0/1 | 1/5 |
| HA-MC | 0/2 | 0/1 | 0/1 | | 0/4 |

The ability of composite hydrogels formed by crosslinking of an HA derivative and a cellulose derivative to inhibit adhesions in the rabbit abrasion model described in Example 3 was also tested. Briefly, HA-ADH was administered together with CMC—CHO, MC—CHO, or HPMC—CHO to form 2 wt % HA-CMC, HA-MC, HA-HPMC hydrogels. Saline injection was used as a control. As shown in Table 11, HA-CMC, HA-MC, and HA-HPMC showed a good peritoneal adhesion preventive effect.

TABLE 11
Results of rabbit tests (4 rabbits per group)

| | HA- | HA- | HA- | Control |
|---|---|---|---|---|
| | CMC | HPMC | MC | (Saline) |
| % Weight change | −6.5 ± 3.6 | −2.8 ± 2.6 | −8.4 ± 3.3 | −11.7 ± 2.4 |
| Score 3 | 1 | 2 | 0 | 3 |
| Score 2 | 1 | 0 | 0 | 1 |
| Score 1 | 0 | 0 | 1 | 0 |
| No adhesion | 2 | 2 | 3 | 0 |
| Median adhesion score | 1 | 2 | 0 | 3 |
| Adhesion area (cm²) | 2.2 ± 3.3 | 0.3 ± 0.6 | 0.0 ± 0.0 | 13.1 ± 1.9 |

### Example 12

### Prevention of Peritoneal Adhesions by In Situ Cross-Linking Hydrogels of Hyaluronic Acid (HA) and Cellulose Derivatives

Introduction

Postoperative peritoneal adhesions can cause pelvic pain, bowel obstruction and infertility (DiZerega, G. S., Peritoneal Surgery. 1999.New York: Springer; incorporated herein by reference). A number of membranous barrier devices have been developed commercially, with varying degrees of success (DiZerega, G. S., Peritoneal Surgery. 1999. New York: Springer; incorporated herein by reference). Gels that form in situ by simple mixing of two different polymers are appealing for this purpose as they are easy to handle at room temperature and do not require a radiant light source or toxic chemical cross-linkers (Johns, D. B.; Rodgers, K. E.; Donahue, W. D.; Kiorpes, T. C.; diZerega, G. S., Reduction of adhesion formation by postoperative administration of ionically cross-linked hyaluronic acid. Fertil Steril 1997;68(1):37-42; Li, H.; Liu, Y. C.; Shu, X. Z.; Gray, S. D.; Prestwich, G. D., Synthesis and biological evaluation of a cross-linked hyaluronan-mitomycin C hydrogel. Biomacromolecules 2004;5(3):895-902; Liu, Y. C.; Li, H.; Shu, X. Z.; Gray, S. D.; Prestwich, G. D., Crosslinked hyaluronan hydrogels containing mitomycin C reduce postoperative abdominal adhesions. Fertil Steril 2005;83:1275-1283; Oh, S. H.; Kim, J. K.; Song, K. S.; Noh, S. M.; Ghil, S. H.; Yuk, S. H.; Lee, J. H., Prevention of postsurgical tissue adhesion by anti-inflammatory drug-loaded pluronic mixtures with sol-gel transition behavior. J Biomed Mater Res A 2005;72(3):306-16; Yeo, Y.; Highley, C.; Bellas, E.; Ito, T.; Marini, R.; Langer, R.; Kohane, D., In situ cross-linkable hyaluronic acid hydrogels prevent post-operative abdominal adhesions in a rabbit model. Biomaterials 2006;27:4698-4705; Bulpitt, P.; Aeschlimann, D., New strategy for chemical modification of hyaluronic acid: Preparation of functionalized derivatives and their use in the formation of novel biocompatible hydrogels. J Biomed Mater Res 1999;47(2):152-169; Jia, X. Q.; Colombo, G.; Padera, R.; Langer, R.; Kohane, D. S., Prolongation of sciatic nerve blockade by in situ cross-linked hyaluronic acid. Biomaterials 2004;25(19):4797-4804; each of which is incorporated herein by reference). They are generally easier to apply over the injured areas, especially if those are difficult to cover with simple sheets, or if the area is very large.

Hyaluronic acid (HA) is a good candidate material for such an application (Johns, D. B.; Rodgers, K. E.; Donahue, W. D.; Kiorpes, T. C.; diZerega, G. S., Reduction of adhesion formation by postoperative administration of ionically cross-linked hyaluronic acid. Fertil Steril 1997;68(1):37-42; Li, H.; Liu, Y. C.; Shu, X. Z.; Gray, S. D.; Prestwich, G. D., Synthesis and biological evaluation of a cross-linked hyaluronan-mitomycin C hydrogel. Biomacromolecules 2004;5(3):895-902; Liu, Y. C.; Li, H.; Shu, X. Z.; Gray, S. D.; Prestwich, G. D., Crosslinked hyaluronan hydrogels containing mitomycin C reduce postoperative abdominal adhesions. Fertil Steril 2005;83:1275-1283; Yeo, Y.; Highley, C.; Bellas, E.; Ito, T.; Marini, R.; Langer, R.; Kohane, D., In situ cross-linkable hyaluronic acid hydrogels prevent post-operative abdominal adhesions in a rabbit model. Biomaterials 2006;27:4698-4705; Bulpitt, P.; Aeschlimann, D., New strategy for chemical modification of hyaluronic acid: Preparation of functionalized derivatives and their use in the formation of novel biocompatible hydrogels. J Biomed Mater Res 1999;47(2):152-169; Jia, X. Q.; Colombo, G.; Padera, R.; Langer, R.; Kohane, D. S., Prolongation of sciatic nerve blockade by in situ cross-linked hyaluronic acid. Biomaterials 2004;25(19):4797-4804; each of which is incorporated herein by reference), because HA is well-known to be biocompatible in the peritoneum (DiZerega, G. S., Peritoneal Surgery. 1999.New York: Springer; incorporated herein by reference), and chemically cross-linked HA hydrogels (HAX) can prevent peritoneal adhesions in a rabbit model (Yeo, Y.; Highley, C.; Bellas, E.; Ito, T.; Marini, R.; Langer, R.; Kohane, D., In situ cross-linkable hyaluronic acid hydrogels prevent post-operative abdominal adhesions in a rabbit model. Biomaterials 2006;27:4698-4705; incorporated herein by reference). Hyaluronic acid is degraded by endogenous hyaluronidase (Knepper, P. A.; Farbman, A. I.; Telser, A. G., Exogenous Hyaluronidases And Degradation Of Hyaluronic-Acid In The Rabbit Eye. Investigative Ophthalmology and Visual Science 1984;25(3):286-293; incorporated herein by reference) and by hydroxyl radicals (Soltes, L.; Mendichi, R.; Kogan, G.; Schiller, J.; Stankovska, M.; Arnhold, J., Degradative action of reactive oxygen species on hyaluronan. Biomacromolecules 2006;7(3):659-668; Yui, N.; Okano, T.; Sakurai, Y., Inflammation Responsive Degradation Of Cross-Linked Hyaluronic-Acid Gels. J Control Rel 1992;22(2):105-116; each of which is incorporated herein by reference). We have shown that the HAX gels degraded substantially within a week, leaving a significantly reduced amount of gels in the peritoneum (Yeo, Y.; Highley, C.; Bellas, E.; Ito, T.; Marini, R.; Langer, R.; Kohane, D., In situ cross-linkable hyaluronic acid hydrogels prevent post-operative abdominal adhesions in a rabbit model. Biomaterials 2006;27:4698-4705; incorporated herein by reference). Depending on the severity or area of injury, however, it may be beneficial to design hydrogels that can last longer in the peritoneum. We hypothesized that hybridization of HA with other biocompatible polysaccharides that are not degraded enzymatically in humans could slow degradation while preserving HA's excellent biocompatibility. Cellulose derivatives such as carboxymethylcellulose (CMC) (Elkins, T. E.; Bury, R. J.; Ritter, J. L.; Ling, F. W.; Ahokas, R. A.; Homsey, C. A.; Malinak, L. R., Adhesion Prevention By Solutions Of Sodium Carboxymethylcellulose In The Rat.1. Fertil Steril 1984;41(6):926-928; Liu, L. S.; Berg, R. A., Adhesion barriers of carboxymethylcellulose and polyethylene oxide composite gels. J Biomed Mater Res 2002;63(3):326-332; Lehr, C. M.; Bouwstra, J. A.; Schacht, E. H.; Junginger, H. E., Invitro Evaluation Of Mucoadhesive Properties Of Chitosan And Some Other Natural Polymers. International Journal of Pharmaceutics 1992;78(1):43-48; Leach, R. E.; Burns, J. W.; Dawe, E. J.; SmithBarbour, M. D.; Diamond, M. P., Reduction of postsurgical adhesion formation in the rabbit uterine horn model with use of hyaluronate/carboxymethylcellulose gel. Fertil Steril 1998;69(3):415-418; each of which is incorporated herein by reference) and hydroxypropylmethyl cellulose (HPMC) (Lehr, C. M.; Bouwstra, J. A.; Schacht, E. H.; Junginger, H. E., In vitro Evaluation Of Mucoadhesive Properties Of Chitosan And Some Other Natural Polymers. International Journal of Pharmaceutics 1992;78(1):43-48; incorporated herein by reference) are also known to have good biocompatibility in the peritoneum. The biocompatibility of methyl cellulose (MC) in the peritoneum is not known, but the mixture of MC and HA has been reported to be biocompatible in intrathecal injection (Gupta, D.; Tator, C. H.; Shoichet, M. S., Fast-gelling injectable blend of hyaluronan and methylcellulose for intrathecal localized delivery to the injured spinal cord. Biomaterials 2006;27:2370-2379; incorporated herein by reference).

We synthesized in situ cross-linking injectable hydrogels composed of HA and cellulose derivatives such as CMC, HPMC and MC. We characterized these hydrogels in vitro, studied their cytotoxicity in cell culture, and their biocompatibility in the murine peritoneum. Finally, we studied their effectiveness in preventing peritoneal adhesions in a rabbit model.

Materials and Methods

Synthesis of the Polymers and Hydrogels

Reagents: HA (M_{w}=490 kDa and 1.4 MDa) was purchased from Genzyme (Cambridge, Mass.). CMC (Product No: C4888), HPMC (Product No: H9262), MC (Product No: M0387), hyaluronidase, adipic dihydrazide (ADH), 1-ethyl-3-[3-(dimethylamino)propyl]-carbodiimide (EDC), hydroxybenzotriazole (HOBt), sodium periodate, ethylene glycol, tert-butyl carbazate (t-BC), sodium bicarbonate, sodium chloride, and acetic acid were purchased from Sigma-Aldrich (St.Louis, Mo.). Pullulans purchased from Showa Denko (Japan) were used as standards for gel permeation chromatography (GPC).

Preparation of aldehyde polymers: 1.4 MDa HA, CMC, HPMC, and MC were modified to aldehyde forms (HA-CHO, CMC—CHO, HPMC—CHO, and MC—CHO respectively), as shown in FIG. 12. The protocol was similar as that previously used for HA-CHO (Kohane D S, Lipp M, Kinney R C, Anthony D C, Louis D N, Lotan N, et al. Biocompatibility of lipid-protein-sugar particles containing bupivacaine in the epineurium. J Biomed Mater Res 2002;59(3):450-459; Kohane D S, Tse J Y, Yeo Y, Padera R, Shubina M, Langer R. Biodegradable polymeric microspheres and nanospheres for drug delivery in the peritoneum. J Biomed Mater Res 2005:In press; Orita H, Fukasawa M, Girgis W, diZerega G S. Inhibition of postsurgical adhesions in a standardized rabbit model: intraperitoneal treatment with tissue plasminogen activator. Int J Fertil 1991;36(3):172-177, each of which is incorporated herein by reference). Briefly, 1.5 g of HA, CMC, HPMC, or MC was dissolved in 150 ml water, then 802 mg sodium periodate were added, and stirred for 2 h. 200 μl ethylene glycol was added to stop the reaction, and the mixture was dialyzed immediately against water. The purified product was freeze dried and kept at 4° C.

Preparation of hydrazide polymer: 490 kDa HA was modified into adipic dihydrazide HA (HA-ADH) using a previously described method (Yeo, Y.; Highley, C.; Bellas, E.; Ito, T.; Marini, R.; Langer, R.; Kohane, D., In situ cross-linkable hyaluronic acid hydrogels prevent post-operative abdominal adhesions in a rabbit model. Biomaterials 2006;27:4698-4705; Bulpitt, P.; Aeschlimann, D., New strategy for chemical modification of hyaluronic acid: Preparation of functionalized derivatives and their use in the formation of novel biocompatible hydrogels. J Biomed Mater Res 1999;47(2):152-169; Jia, X. Q.; Colombo, G.; Padera, R.; Langer, R.; Kohane, D. S., Prolongation of sciatic nerve blockade by in situ cross-linked hyaluronic acid. Biomaterials 2004;25(19):4797-4804; each of which is incorporated herein by reference).

Preparation of disk hydrogels: 2 wt % HA-ADH in PBS and 2 wt % HA-CHO, CMC—CHO, HPMC—CHO, or MC—CHO in PBS were injected into a rubber mold sandwiched between two glass slides using a double-barreled syringe (Baxter, Deerfield, Ill.). The diameter and the thickness of the prepared hydrogel were 1.2 cm and 3.5 mm, respectively. Below, these cross-linked hydrogels are termed HAX, HA-CMC, HA-HPMC, and HA-MC, respectively.

Characterization of Polymers and Hydrogels

Characterization of polymers: ¹H-NMR (Varian: Unity 300 spectrophotometer, Palo Alto, Calif.) spectroscopy of 10 mg/ml solutions of HA-ADH, HA-CHO, CMC—CHO, HPMC—CHO and MC—CHO in D₂O was performed. The aldehyde polymers (HA-CHO, CMC—CHO, HPMC—CHO, and MC—CHO) were analyzed after reacting with t-BC as described in Jia, X. Q.; Colombo, G.; Padera, R.; Langer, R.; Kohane, D. S., Prolongation of sciatic nerve blockade by in situ cross-linked hyaluronic acid. Biomaterials 2004;25(19):4797-4804. ¹H-NMR spectra of the polymers reacted to t-BC were measured in D₂O.

The molecular weights of the polysaccharides were measured using GPC. The column was Ultrahydrogel Linear (Waters, Milford, Mass.), and refractive index (RI) was detected by refractometer (Wyatt Technology: OPTILAB DSP, Santa Barbara, Calif.). Mobile phase was the mixture of 0.05 M sodium and 0.2 M sodium chloride (pH=6.7), and its flow rate was 0.8 ml/min. Pullulans (Shodex, Pullulan Standards P5-P800, Japan) were used as molecular weight standards.

Characterization of hydrogels: Gelation time was measured by the following protocol. One hundred microliters of aqueous HA-CHO, CMC—CHO, HPMC—CHO, or MC—CHO solution were added to 100 μl of aqueous HA-ADH solution which was mixed with a magnetic stir bar on a petri dish at 155 rpm using a hot plate/stirrer (Coming: Model PC-320, Coming, N.Y.). The gelation time was the time until the mixture became a globule; it was measured 4 times per sample.

Shear moduli of the prepared disk gel were measured with a rheometer (TA Instruments: AR1000, New Castle, Del.). The disk gels were immersed in PBS for 5 days and allowed to swell to equilibrium. Creep and relaxation tests were done at different shear stresses. Shear was applied for 3 min, followed by 3 min relaxation. The strain values reached constancy during the creep tests, and then returned to zero during the relaxation tests in each measurement. Shear modulus, G, was calculated from the slope of the linear relationship between stress and strain. R² values of fitted lines between stress and strain were above 0.95.

The time course of swelling of the gel disks was measured gravimetrically in PBS at 37° C. The weight of hydrogel after gelation, WS, was measured after immersion in PBS for 5 days. The swelling ratio, Q, of Wₛ to the initial weight of hydrogel right after the gelation, Wᵢ, was calculated as Q=Wₛ/Wᵢ.

Degradation kinetics was measured as follows: Four hydrogel discs were incubated in 10 unit/ml hyaluronidase in PBS at 37° C. At each time point, the gel disks were weighed, and the hyaluronidase solution was replaced. Measurements were made over 14 days. The ratio of the volume of hydrogels at each time point to the initial volume was determined (volume of the hydrogel (%)).

Cytotoxicity Assay

In vitro cell viability in the presence of HA-CHO, CMC—CHO, MC—CHO, and HPMC—CHO were investigated by the MTT assay (Promega, Madison, Wis.) using a human mesothelial cell line (ATCC: CRL-9444, Manassas, Va.) and macrophage cell line J774.A1 (ATCC: TIB-67™).

Mesothelial cells were grown and maintained in a complete growth medium (GIBCO: Medium199 with Earle's BSS, 0.75 mM L-glutamine and 1.25 g/L sodium bicarbonate supplemented with with 3.3 nM epidermal growth factor, 400 nM hydrocortisone, 870 nM insulin, 20 mM HEPES and 10% fetal bovine serum) at 37° C. in 5% CO₂. Macrophages were grown and maintained in DMEM media (GIBCO: DMEM Cat #10569-010 with 10% fetal bovine serum). 5×10⁴ cells were placed in each well of a 24-well plate, and incubated at 37° C. in 5% CO₂ overnight, then media were replaced with media containing different concentration of HA-B, CMC—B, HPMC—B, and MC—B. On the third day after adding those materials in the case of mesothelial cells, or the second day in the case of J774.A1cells, MTT assays were performed. One hundred μl of tetrazolium salt solution was added into each well and incubated at 37° C. for 4 h. The purple formazan produced by active mitochondria was solubilized using 1 ml detergent solution and then read at 570 nm by plate reader (Molecular Devices: SpectraMax 384, Union City, Calif.). The absorbance values were normalized to wells in which cells were not treated with polymers.

In Vivo Experiments

All the animals were cared for in compliance with protocols approved by the Animal Care and Use Committee at the Massachusetts Institute of Technology, and the Principles of Laboratory Animal Care (NIH publication #85-23, revised 1985).

Injections of Hydrogels into Mouse Peritoneum

SV129 mice weighing 25 g were purchased from Taconic (Hudson, N.Y.), and housed in groups in a 6 AM-6 PM light-dark cycle.

The polymers were sterilized by UV irradiation for 2 hours, then dissolved in saline at 2 wt % concentration. Anesthesia was induced with 50 mg/kg ketamine and 10 mg/kg xylazine, and a 5 mm skin incision was made, revealing the translucent abdominal wall. A 24 gauge catheter (Terumo: Surflash I.V. Catheter, Japan) was placed through the abdominal wall, and 0.3 ml of air was insufflated to confirm positioning. The catheter was then advanced 1 cm, and 0.5 ml aldehyde polysaccharide (HA-CHO, CMC—CHO, HPMC—CHO, or MC—CHO) and 0.5 ml HA-ADH were injected using a dual syringe applicator (Baxter: Deerfield, Ill.).

The mice were sacrificed after 4 days, 1 week, 2 weeks and 3 weeks after the injections, and the presence of residue and adhesions were evaluated. The dissector was blinded as to which treatment individual mice had received. Abdominal contents were sampled as needed were sampled, fixed in 10% formalin, and processed for histology (hematoxylin-eosin stained slides) using standard techniques.

Evaluation of Peritoneal Adhesion-Preventing Effect by a Rabbit Sidewall Defect-Bowel Abrasion Model

Peritoneal adhesions were induced as described (Yeo et al., In situ cross-linkable hyaluronic acid hydrogels prevent post-operative abdominal adhesions in a rabbit model. Biomaterials 2006;27:4698-4705; incorporated herein by reference). Female albino rabbits (Oryctolagus cuniculus; New Zealand White, Covance, Hazleton, Pa.) (3±0.5 kg) were anesthetized using ketamine (35 mg/kg i.m.) and xylazine (5 mg/kg i.m.); maintenance was achieved using 1-3% isoflurane in balance oxygen. A 10 cm long midline incision was made along the linea alba, and the peritoneum was opened. Peritoneal adhesions were induced by making a 3×4 cm defect on the right lateral abdominal wall and abrading seven haustra of the cecum until a bleeding surface was obtained.

Four animals were assigned randomly to each experimental group: (i) saline; (ii) covering the excised abdominal wall and abraded cecal surface with 10 ml of cross-linked HA-CMC, HA-HPMC, or HA-MC. Prior to application, the materials were sterilized by germicidal UV illumination for 2 hours and dissolved in sterile saline. The gel precursor solutions (5 ml of HA-ADH (20 mg/ml) and 5 ml of CMC—CHO, HPMC—CHO or MC—CHO (20 mg/ml)) were placed in separate sterile 10 ml syringes, which were connected to a dual syringe applicator, and co-extruded through a 15 gauge needle. The liquid precursors started to gel instantly, conforming to the shape of the target area.

Post-surgical animal care was delivered as described previously (Yeo, Y.; Highley, C.; Bellas, E.; Ito, T.; Marini, R.; Langer, R.; Kohane, D., In situ cross-linkable hyaluronic acid hydrogels prevent post-operative abdominal adhesions in a rabbit model. Biomaterials 2006;27:4698-4705; incorporated herein by reference). One week after the procedure, animals were euthanized with sodium pentobarbital 100 mg/kg IV. Adhesions were scored using a reported method: Score 0=no adhesion, score 1=tissue adherence that would separate with gravity, score 2=tissue adherence separable by blunt dissection, score 3=adhesion requiring sharp dissection. The area of adhesions with scores of 2 and 3 were also measured. Tissues of interest were sampled and prepared for histology as described above.

Statistical Analysis

Data were analyzed by Student t-tests preceded by ANOVAs. Wilcoxon rank-sum tests were done for adhesion scores between each hydrogel and control. Statistical tests were done with KaleidaGraph® (Synergy Software). A p-value<0.05 was considered statistically significant.

Results

Synthesis and Characterization of HA-ADH, HA-CHO, CMC—CHO, HPMC—CHO, and MC—CHO

Synthesis of HA-ADH was confirmed by the methylene protons of the adipic dihydrazide (singlet peak at 1.62 ppm and doublet peak at 2.25 ppm and 2.38 ppm) (Yeo, Y.; Highley, C.; Bellas, E.; Ito, T.; Marini, R.; Langer, R.; Kohane, D., In situ cross-linkable hyaluronic acid hydrogels prevent post-operative abdominal adhesions in a rabbit model. Biomaterials 2006;27:4698-4705; Bulpitt, P.; Aeschlimann, D., New strategy for chemical modification of hyaluronic acid: Preparation of functionalized derivatives and their use in the formation of novel biocompatible hydrogels. J Biomed Mater Res 1999;47(2):152-169; Jia, X. Q.; Colombo, G.; Padera, R.; Langer, R.; Kohane, D. S., Prolongation of sciatic nerve blockade by in situ cross-linked hyaluronic acid. Biomaterials 2004;25(19):4797-4804; each of which is incorporated herein by reference). The degree of modification was calculated from the ratio of the area of the peak for N-acetyl-D-glucosamine residue of HA (singlet peak at 2.0 ppm) to that for the methylene protons of the adipic dihydrozide at 1.62 ppm; the degree of modification was 48.4%.

For analysis of aldehyde groups formed by the oxidation reaction, the aldehyde polymers were reacted with t-BC prior to ¹H-NMR analysis. In each of the aldehyde-modified polysaccharides, the chemical shifts of t-butyl groups appeared (single peak at 1.20 ppm and single peak at 1.43 ppm), indicating the successful syntheses of HA-CHO, CMC—CHO, HPMC—CHO, and MC—CHO. The M_{w} and M_{w}/Mₙ of HA were 1432 kDa and 5.2. The M_{w}s of CMC, HPMC, and MC were >1 MDa. The M_{w}s of the aldehyde polymers were between 109 and 239 kDa, which were lower than that of HA-ADH (Table 12.1).

TABLE 12.1
Molecular weights of modified polymers

| | M_{w} | Mₙ | |
|---|---|---|---|
| | (kDa) | (kDa) | M_{w}/Mₙ |
| HA-ADH* | 1502 | 108 | 13.9 |
| HA-CHO | 239 | 65 | 3.7 |
| CMC-CHO | 128 | 46 | 2.8 |
| HPMC-CHO | 109 | 23 | 4.8 |
| MC-CHO | 162 | 33 | 4.9 |

*Previously reported (Kohane DS, Lipp M, Kinney RC, Anthony DC, Louis DN, Lotan N, et al. Biocompatibility of lipid-protein-sugar particles containing bupivacaine in the epineurium. J Biomed Mater Res 2002; 59(3): 450-459, which is incorporated herein by reference).

M_{w}: weight-averaged molecular weight, Mₙ:number-averaged molecular weight

In Vitro Physicochemical Properties of In-Situ Hydrogels

HA-ADH and the aldehyde-modified cellulose derivatives all formed gels within an acceptably brief time frame (Table 12.2). The gelation time of HA-CMC was significantly longer than the rest (p<0.0001 between HA-CMC and other aldehyde polysaccharides).

TABLE 12.2
Physical properties of cross-linked hydrogels

| | Gelation time | | G* |
|---|---|---|---|
| | (sec) | Swelling ratio* (%) | (Pa) |
| HAX | 3.5 ± 1.0 | 200 ± 14 | 32 ± 17 |
| HA-CMC | 19.0 ± 1.7 | 230 ± 10 | 92 ± 19 |
| HA-HPMC | 4.0 ± 1.2 | 120 ± 13 | 292 ± 109 |
| HA-MC | 5.8 ± 2.9 | 150 ± 4 | 297 ± 41 |

*Measured on day 5 of immersion in phosphate buffered saline
Date are averages ± standard deviations (n = 4)

The hydrogels swelled, reached equilibrium one day after immersion in PBS, and remained constant for the following 4 days (data not shown). Throughout, the swelling ratios (Table 12.2) were ordered as follows: HAX, HA-CMC>HA-MC (p<0.001) >HA-HPMC (p=0.0053).

The shear modulus (Table 12.2), G, of HAX was lower than those of HA-CMC, HA-HPMC, or HA-MC (p<0.05). The shear modulus of HA-CMC was lower than those of HA-HPMC or HA-MC (p<0.05). There was no statistical difference in G between HA-MC and HPMC (p=0.93).

Degradation Kinetics in Hyaluronidase Solution

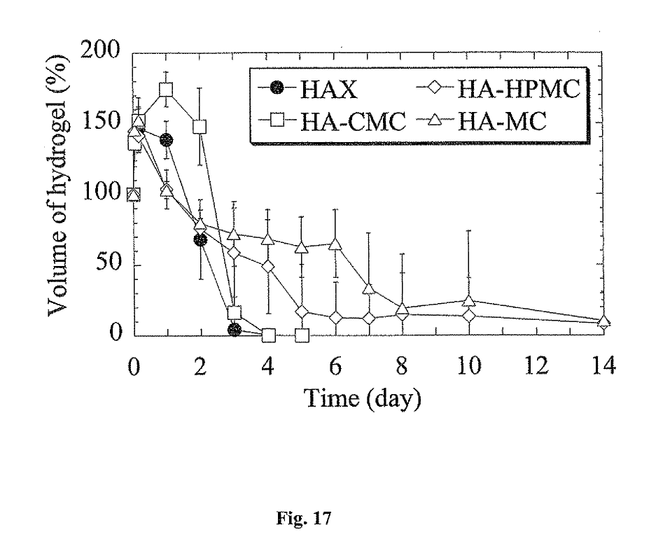

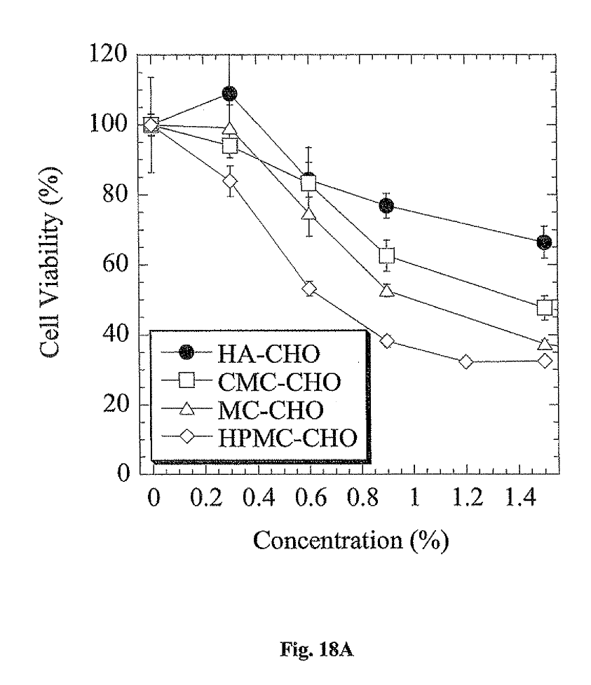

There were differences in the degradation kinetics of the hydrogels in hyaluronidase solution (FIG. 17). HAX degraded the most rapidly (p<0.001 vs. HA-CMC on days 1 and 2). HAX and HA-CMC were completely degraded by the 4th and 5th days, respectively. In contrast, HA-MC and HA-HPMC did not degrade completely for 2 weeks. One possible reason for these differences is that HA-MC and HA-HPMC are more cross-linked than HAX. Another reason could be that hyaluronidase did not diffuse as effectively into HA-MC and HA-HPMC because they did not swell as much as HAX and HA-CMC.

The Effect of Polymers on the Viability of Mesothelial Cells and Macrophages

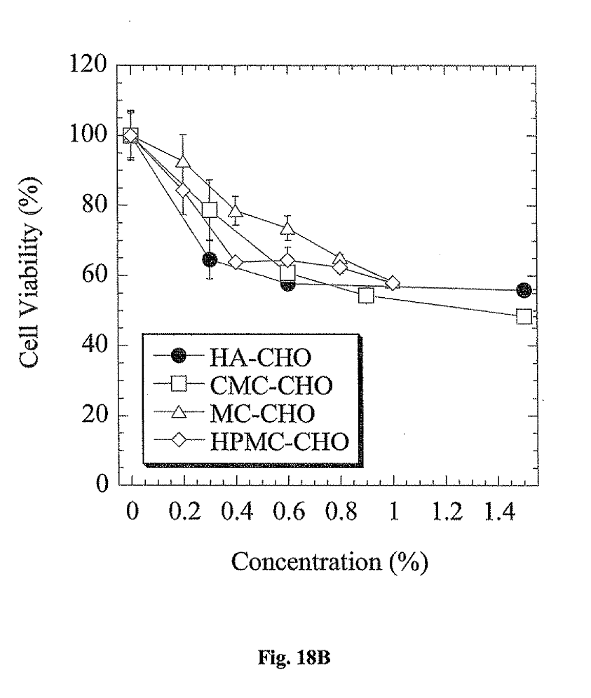

Mesothelial cells were cultured in the presence of a range of concentrations of aldehyde polymers. There was a dose-dependent reduction in cell viability for all polymers. Cell viability was not reduced by HA-CHO and MC—CHO at 0.3% (w/v) (p>0.05) (FIG. 18A), while HPMC—CHO (p=0.0011) and CMC—CHO (p=0.044) caused a small reduction in cell viability. At higher concentrations, HA-CHO showed a small decrease in cell viability, while the cellulose derivatives showed more: the rank order of cell viability after 3 days of incubation was HA-CHO>CMC—CHO>MC—CHO>HPMC—CHO (p<0.01 at any pair at 0.9% (w/v)).

Aldehyde-modified polymers also showed a dose-dependent effect on cell viability in macrophages (FIG. 18B). Here, the difference in cell viability between HA and cellulose derivatives was not seen. Although there were some statistical differences between compounds at some concentrations, on the whole cell viability was similar between groups.

Biocompatibility of the In Situ Hydrogels in the Mouse Peritoneum

Mice (n=4 to 6) were injected with 1 ml of gel precursors. Animals were sacrificed at predetermined intervals over the next three weeks to assess adhesion formation (Table 12.3). In all twenty animals, there was only one adhesion between the bladder and other viscera. Given the presence of scar and clot at the site of the adhesion, it was felt to be due to direct trauma during the injection of gel.

TABLE 12.3
Adhesions following intraperitoneal injection of hydrogels in mice

Days to dissection
post-surgery

| Hydrogel | 4 | 7 | 14 | 21 | Total |
|---|---|---|---|---|---|
| HAX | 0/2 | 0/1 | 0/1 | 0/1 | 0/5 |
| HA-CMC | 0/3 | 0/1 | 0/1 | 0/1 | 0/6 |
| HA-HPMC | 0/2 | 0/1 | 1/1 | 0/1 | 1/5 |
| HA-MC | 0/2 | 0/1 | 0/1 | — | 0/4 |

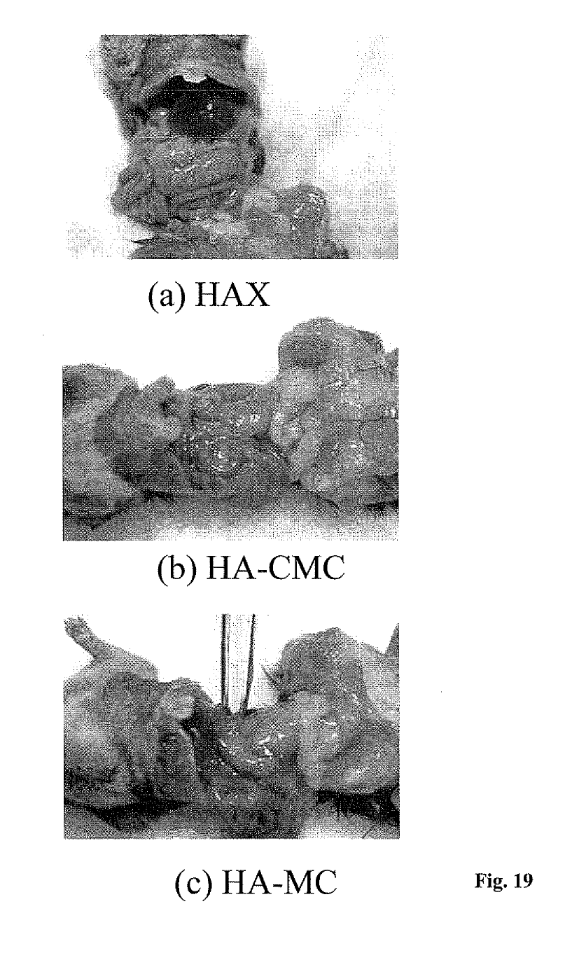

It was not possible to quantitate the amount of residual gel in the abdominal cavity. On gross examination, there appeared to be much more hydrogel residue in animals injected with HA-MC than in the others (FIG. 19). After 3 weeks, HAX had completely disappeared, and a small volume of HA-HPMC was found as a discrete gel. HA-CMC persisted only as a thin layer covering the viscera. Histology of the peritoneum and viscera was normal in all samples.

Prevention of Peritoneal Adhesions

Adhesions were induced in rabbits by abrasion of the cecum and excision of a section of adjacent abdominal wall (Table 12.4). In control animals, saline was applied instead. All animals in the saline group developed adhesions over a large area (FIG. 19B). The area of adhesions was greatly reduced in groups treated with HA-CMC (p=0.001 1), HA-MC (p<0.0001), and HA-HPMC (p<0.0001). There was no statistically significant difference between the gels in that parameter. Statistical significance of the decrease in adhesion scores could only be shown with HA-MC (p=0.023) (FIG. 19C). Of note, in rabbits treated with HA-HPMC two of the score 3 adhesions formed on the incision line on the abdominal midline i.e., outside of the area where the gel was applied. We have described the effectiveness of HAX in preventing peritoneal adhesions elsewhere (Yeo, Y.; Highley, C.; Bellas, E.; Ito, T.; Marini, R.; Langer, R.; Kohane, D., In situ cross-linkable hyaluronic acid hydrogels prevent post-operative abdominal adhesions in a rabbit model. Biomaterials 2006;27:4698-4705).

TABLE 12.4
Effectiveness of hydrogels in preventing
peritoneal adhesions in the rabbit

| | HA- | HA- | HA- | Control |
|---|---|---|---|---|
| | CMC | HPMC | MC | (Saline) |
| Animal weight loss | 6.5 ± 3.6 | 2.8 ± 2.6 | 8.4 ± 3.3 | 11.7 ± 2.4 |
| postoperatively (%) | | | | |
| Score 3 | 1 | 2 | 0 | 3 |
| Score 2 | 1 | 0 | 0 | 1 |
| Score 1 | 0 | 0 | 1 | 0 |
| No adhesion | 2 | 2 | 3 | 0 |
| Median adhesion score | 1 | 2 | 0 | 3 |
| Adhesion area (cm²) | 2.2 ± 3.3 | 0.3 ± 0.6 | 0.0 ± 0.0 | 13.1 ± 1.9 |

Animal weight loss refers to loss in the week following surgery.
Adhesion area is the total area of adhesions with scores of 2 and 3.
Weight change and adhesion area are expressed as average ± standard deviation (n = 4 per group).

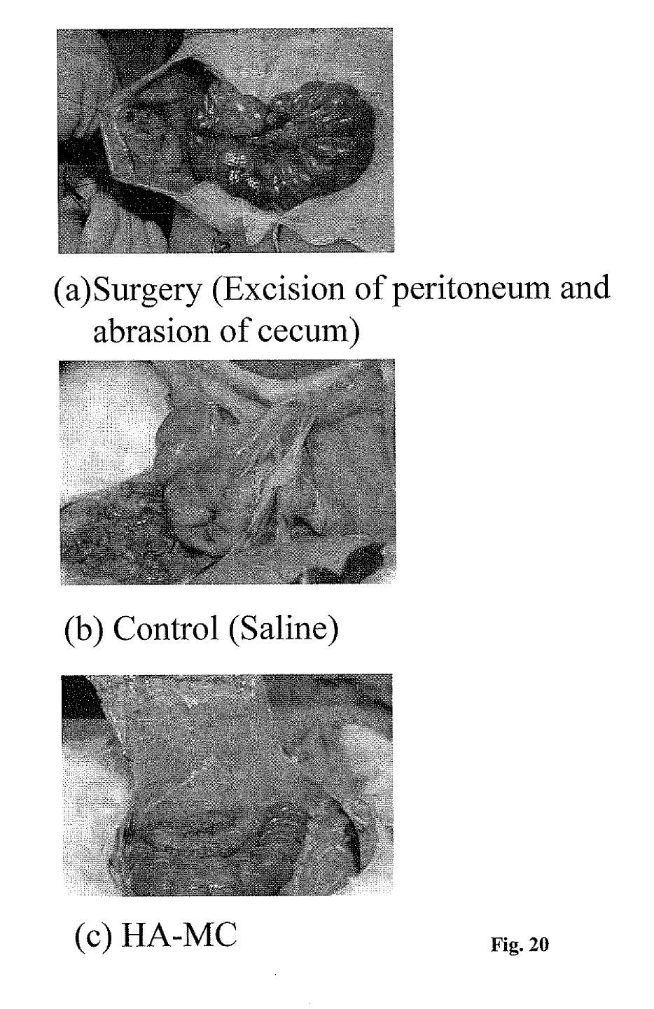

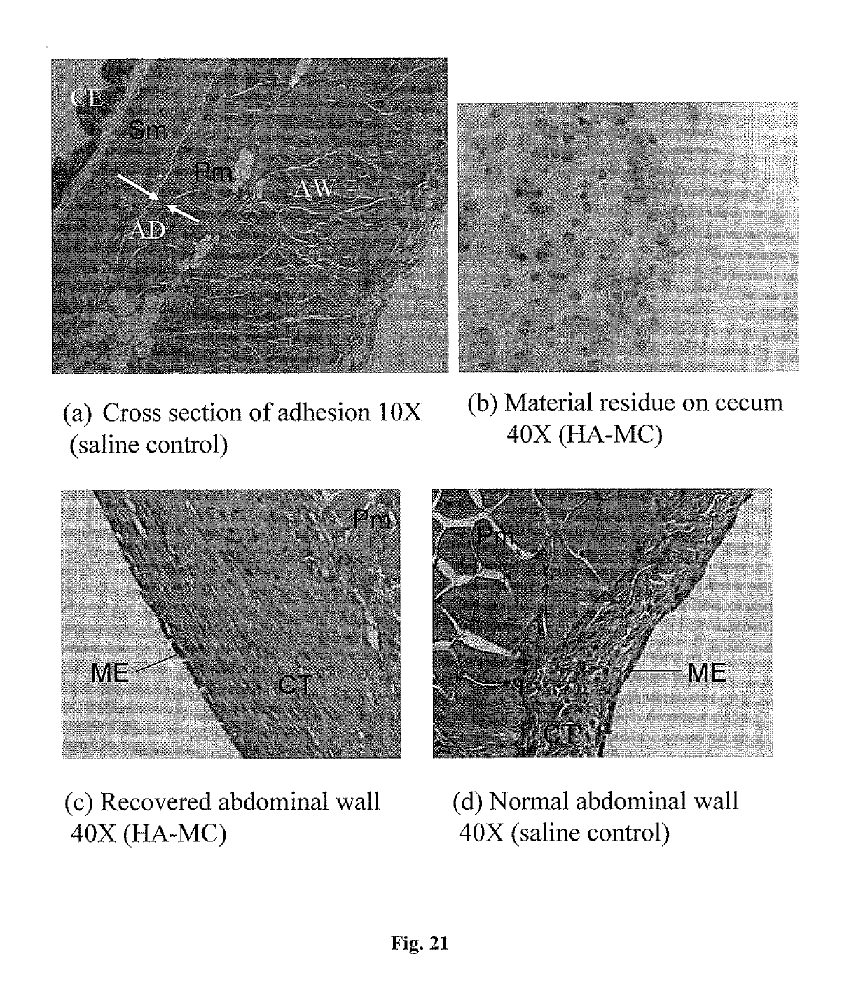

Histological analysis of the adhesion site in the saline-treated animals showed fibroblasts and inflammatory cells in the tissue connecting the cecum and abdominal wall (FIG. 20A). In animals treated with cross-linkable gels, neutrophils and macrophages were found in the hydrogel residues (FIG. 20B). Where adhesions were prevented, the site of injury was re-epithelialized, (FIG. 20C), although fibroblasts were still prominent in the subjacent layers compared to the normal abdominal wall (FIG. 20D). Similar results were observed with all three cellulose derivatives.

Discussion

The hybrid HA-cellulose derivative hydrogels presented here were suitable for use in the peritoneum. Their physicochemical properties including gelation time, mechanical strength, water content, swelling kinetics, and degradation kinetics were appropriate to the anticipated use. This was confirmed by good handling properties during surgery, and by biological outcomes. Although the precursor polymers showed some cytotoxicity in vitro, there was no apparent local toxicity in vivo. One possible explanation is that the rapid cross-linking leaves little free precursor. The benign nature of these formulations was shown by their biocompatibility in the murine and rabbit models, although long-term safety and efficacy remain to be demonstrated. Finally, the hydrogels showed a marked effect in reducing adhesion formation. We note that many of the adhesions that occurred in the rabbits were located outside of the areas in which the treatments were applied. Therefore, an important unanswered question is whether these materials are best applied in a manner restricted to the site of injury, as done here, or more broadly, or throughout the peritoneum.

There are commercially available materials for the prevention of peritoneal adhesions that have related compositions of matter. SEPRAFILM® (Genzyme) is a preformed hydrogel sheet of HA and CMC crosslinked with 1-(3-dimethylaminopropyl)-3-ethylcarbodiimide hydrochloride (Diamond et al., Reduction of adhesions after uterine myomectomy by Seprafilm membrane (HAL-F): A blinded, prospective, randomized, multicenter clinical study. Fertility And Sterility 1996;66(6):904-910; Kling, J., Genzyme's Seprafilm gets FDA marketing nod. Nature Biotechnology 1996;14(5):572-572; each of which is incorporated herein by reference). INTERCEED® (Johnson & Johnson) is a preformed sheet of oxidized regenerative cellulose (Pagidas, K.; Tulandi, T., Effects Of Ringer Lactate, Interceed(Tc7) And Gore-Tex Surgical Membrane On Postsurgical Adhesion Formation. Fertility And Sterility 1992;57(1):199-201; incorporated herein by reference). There are differences between those devices and ours in the chemistry of cross-linking, the molecules released by that cross-linking (SEPRAFILM® releases carbodiimide, while the materials described here release water), and composition of matter. However, the most significant difference is that these materials must be applied as solid sheets, while the formulations presented here form by in situ gelation without using cross-linking agents.

Hydrogels that cross-link in situ have advantages over conventional barrier devices in terms of ease of applicability, the types of devices through which they can be applied, and perhaps the type of surface that can be treated. The materials presented here have a range of physicochemical properties. The use of hydrazide versions of the cellulose derivatives (instead of HA-ADH) would further affect properties, e.g., by making the resulting gels even more resistant to enzymatic hydrolysis. We also note that there is a considerable difference in cost between the cellulose derivatives and hyaluronic acid.

Of the gels tested here, HA-MC gel was the most effective in preventing peritoneal adhesions. This could be related to the fact that HA-MC degraded more slowly than HA-CMC and HAX in vitro in hyaluronidase, an enzyme present in peritoneum, and thus had a more prolonged barrier effect. Differences in the effectiveness of the various hydrogels preventing adhesions could be due to differences in unsuspected intrinsic biological activities, as may be the case for HA. Effectiveness in preventing adhesions could be changed by further optimizing the physicochemical properties of the gels.

We examined physicochemical properties of the hydrogels, including gelation time, mechanical strength, water content, swelling kinetics, and degradation kinetics. These properties are interrelated, and depend in large part on the properties of the pre-polymers, such as viscosity, electric charge, conformation, solubility, degree of modification, concentration in solution, ease of mixing, and others. Difference in performance between the various polymers probably are due to differences in these parameters, but our results do not allow us to discern the mechanism.

Given the relationship between swelling ratio, polymer concentration and shear modulus (Anseth, K. S.; Bowman, C. N.; BrannonPeppas, L., Mechanical properties of hydrogels and their experimental determination. Biomaterials 1996,17(17):1647-1657; Peppas, N. A.; Merrill, E. W., Crosslinked Polyvinyl-Alcohol) Hydrogels As Swollen Elastic Networks. J Appl Polym Sci 1977;21(7):1763-1770; each of which is incorporated herein by reference), the physicochemical properties studied above suggest that HA-HPMC and HA-MC might be more highly crosslinked than HAX and HA-CMC. The higher cross-linking density may have resulted in part from the fact that MC—CHO and HPMC—CHO are not anionic, so that there was less electrostatic repulsion between the hydrazide polymer and the aldehyde polymer than in those with HA-CHO and CMC—CHO. This interpretation is consistent with the rapid gelation of HA-MC and HA-HPMC as compared to HA-CMC.

Conclusion

The hybrid hydrogels of HA and cellulose derivatives described here could be applied via a double barreled syringe and cross-linked rapidly, suggesting ease of application in the clinical setting, with both open surgery and laparoscopy. Although the aldehyde-modified cellulose derivatives showed some cytotoxicity in vitro, there was good biocompatibility in the murine peritoneum. These formulations were effective in preventing peritoneal adhesions in a rabbit cecal injury-side wall defect model.

### Example 13

### Dextran-Based In Situ Cross-Linked Injectable Hydrogels to Prevent Peritoneal Adhesions

Introduction

Peritoneal adhesion are serious consequences of abdominal and perlvic surgery, and can cause severe pain, bowel obstruction and infertility. In situ cross-linking gels, that form by mixing of two polymers, are easy to apply in the peritoneum and can be very effective. Biomaterials such as hyaluronic acid (HA), oxidized cellulose, and cellulose derivatives have shown excellent biocompatibility in the peritoneum. Dextrans are another attractive base material for in situ cross-linkable matrices. Dextran (DX) is a polysaccharide where glucose moieties are mainly connected by α-1,6-linkages. 40 kDa and 70 kDa dextrans have been used clinically to prevent vascular occlusion, as a plasma volume expander, and for anti-coagulation therapy. Dextran has proven biocompatibility in the peritoneum. A 32% solution of 70 kDa dextran was used clinically to prevent peritoneal adhesions in the 1980's, but dextrans have fallen into disuse because there were both successful and unsuccessful clinical trials.

In this study, we synthesized novel dextran-based injectable hydrogels for the prevention of peritoneal adhesions. Carboxymethyldextran (CMDX) modified with a hydrazide group (CMDX-ADH), was cross-linked to either DX or CMC modified with an aldehyde group (DX—CHO or CMC—CHO) at room temperature. We characterize the resulting hydrogels in vitro, study the cytotoxicity of the pre-polymer in cell culture, and their effectiveness in preventing peritoneal adhesions in a rabbit sidewall defect-bowel abrasion model.

Materials and Methods

Synthesis of the Polymers and Hydrogels

Reagents: 70 kDa (Product No: D4751), 500 kDa (Product No: D1037), and 2 MDa (Product No: D5376) dextran from Leuconostoc mesenteroides, CMC (Product No: C4888), adipic dihydrazide (ADH), 1-ethyl-3-[3-(dimethylamino)propyl]-carbodiimide (EDC), hydroxybenzotriazole (HOBt), sodium periodate, ethylene glycol, tert-buthyl carbazate (t-BC), chloroacetic acid, sodium hydroxide, sodium chloride, and hydrochloric acid were purchased from Sigma-Aldrich.

Preparation of carboxymethyldextran (CMDX): 70 kDa and 500 kDa dextran were modified into carboxymethyldextran (CMDX) with the previous method [Ying's paper and Bioconjugate 1997]. Briefly, 10 g of dextran was dissolved in 100 ml of distilled water overnight, and then both 24.0 g of sodium hydroxide and 30.2 g of chloroacetic acid were added. The solution was refluxed at 70° C. for 145 min, quickly neutralized to pH=7.0 with 6 N hydrochloric acid, dialyzed against distilled water for 3 days, and then lyophilized. The yield was 75-80%.

Preparation of adipic dihydrazide carboxymethyldextran (CMDX-ADH): 70 kDa and 500 kDa CMDX were modified into adipic dihydrozide CMDX (CMDX-ADH) in the same protocol of adipic dihydrozide hyaluronic acid as previously reported.

Preparation of aldehyde dextran (DX—CHO) and carboxymethylcellulose (CMC—CHO): 2 MDa DX, 70 kDa DX, and CMC were modified into aldehyde 70 kDa DX (70 kDa DX—CHO), 2 MDa DX (2 MDa DX—CHO), and aldehyde CMC (CMC—CHO), respectively. The protocol was same with our previous reports.

Preparation of disk hydrogel: 2 wt % CMDX-ADH PBS-buffer solution and 2 wt % aldehyde polymers such as DX—CHO and CMC—CHO PBS-buffer solution were injected into a rubber mold sandwiched between two slide glasses using a double syringe (Baxter: Deerfield, Ill.). The diameter and the thickness of the prepared hydrogel were 1.2 cm and 3.5 mm, respectively. These disk hydrogels are called CMDX-DX and CMDX—CMC, respectively.

Characterization of Polymers and Hydrogels

Characterization of polymers: ¹H-NMR (Varian Unity 300 spectrophotometer) spectroscopy was used to confirm the synthesis. 10 mg/ml CMDX, CMDX-ADH, DX—CHO, and CMC—CHO in D₂O were measured. Next, 10-fold molar excess of t-BC was added to DX—CHO and CMC—CHO polysaccharide in pure water, and aldehyde groups were reacted with t-BC. After dialyzed against water and lyophilized, ¹H-NMR spectra of the reacted polymers were measured in D₂O. Elementary analysis was performed to determine elemental composition.

FT-IR spectra of CMDX, CMDX-ADH, DX—CHO and CMC—CHO were measured after preparing the KBr tablet of each polymer.

Elemental analysis was performed for CMDX-ADH.

Characterization of hydrogels: Gelation time was measured by the reported protocol. Aqueous 0.1 ml DX—CHO or CMC—CHO solution was added into aqueous 0.1 ml CMDX-ADH solution with stirring using stirring bar on petri dish at 155 rpm using a Corning model PC-320 hot plate/stirrer. The time until the mixture of the solutions became a globule hydrogel was measured 5 times.

The time course of swelling of the prepared disk gels was measured gravimetrically in PBS buffer at 37° C. The weight of hydrogel after gelation, Wₛ, was measured after immersed in PBS buffer for 5 days. The swelling ratio, Q, to the initial weight of hydrogel right after the gelation, Wᵢ, was calculated as Q=Wₛ/Wᵢ, and the pictures of the hydrogels were taken.

Cytotoxicity Assay

In vitro cell viability in the presence of DX, CMC, CMDX-ADH, DX—CHO and CMC—CHO were investigated by MTT assay (Promega) using a human mesothelial cell line (CRL-9444: ATCC) and macrophage cell line J774.A1 (TIB-67™: ATCC). The protocol was same with our previous report [HA-CMC paper]. Briefly, mesothelial cells were grown and maintained in the complete growth medium (Medium199 with Earle's BSS, 0.75 mM L-glutamine and 1.25 g/L sodium bicarbonate supplemented with with 3.3 nM epidermal growth factor, 400 nM hydrocortisone, 870 nM insulin, 20 mM HEPES and 10% fetal bovine serum) at 37° C. in 5% CO₂. Macrophages were grown and maintained in DMEM (Gibco Cat #10569-010) with 10% fetal bovine serum. On third day in mesothelial cells or second day in macrophages after adding the materials, MTT assay was performed. DX—CHO only disturbed MTT assay, thus media was replaced with fresh media right before starting the assay. The values are normalized by the control experiments, which nothing was added to the cells.

Injections of Hydrogels into Mice Peritoneum

The protocol was same with our previous method. (Falk K, Bjorquist P, Stromqvist M, Holmdahl L. Reduction of experimental adhesion formation by inhibition of plasminogen activator inhibitor type 1. British Journal of Surgery 2001;88:286-289; which is incorporated herein by reference). SV129 mice weighing about 25 g were purchased from Taconic (Hudson, N.Y.), and 10 ml of CMDX-DX gel, which composed of 0.5 ml of CMDX-ADH (5% w/v) and 0.5 ml of DX—CHO (2% w/v), was injected into peritoneum through catheter using double syringe (Baxter: Deerfield, Ill.). Laparotomy of the mice was done 2 weeks after the injection. A dissector was blinded to which hydrogel each mouse had been injected. The existence of adhesions was assessed by the dissector.

Evaluation of Peritoneal Adhesion-Preventing Effect by a Rabbit Sidewall Defect-Bowel Abrasion Model

The protocol was same with our previous method. See Example 12. Female albino rabbits (Oryctolagus cuniculus; New Zealand White, Covance, Hazleton, Pa.) (3±0.5 kg) were used as model animals. 12 animals were assigned randomly to experimental groups: 10 ml of (i) 70 kDa-CMDX-DX gel (n=4), (ii) 70 kDa-CMDX—CMC gel (n=4), and (iii) 500 kDa-CMDX—CMC gel (n=4).

Peritoneal adhesions were induced with both a 3×4 cm defect on the right lateral abdominal wall and an abrasion of cecums of seven haustra for 80-160 strokes. The gel precursor solutions (5 ml of CMDX-ADH (50 mg/ml for 70 kDa-CMDX-ADH or 40 mg/ml for 500 kDa-CMDX-ADH) and 5 ml of DX—CHO (25 mg/ml) or CMC—CHO (60 mg/ml)) were placed in separate sterile 10-ml syringes, which were connected to a Baxter dual valve applicator, and co-extruded through a 15-gauge needle.

One week after the procedure, animals were euthanized. Adhesions were scored as following: Score 0=no adhesion, score 1=tissue adherence that would separate with gravity, score 2=tissue adherence separable by blunt dissection, score 3=adhesion requiring sharp dissection. Tissues recovered from the necropsy were fixed in 10% formalin, and stained with hematoxylin and eosin for histological examination.

Statistical analysis. Data were basically analyzed by student t-tests. ANOVAs were performed before t-tests. Wilcoxon rank-sum tests were done for adhesion scores between each hydrogel and control. Category variables were assigned as following: score 0=1, score 1=2, score 2=3, and score3=4. All the statistical tests were done using KaleidaGraph® (Synergy Software). A p value<0.05 was considered statistically significant.

Results

Synthesis and Characterization of CMDX, CMDX-ADH, DX—CHO and CMC—CHO

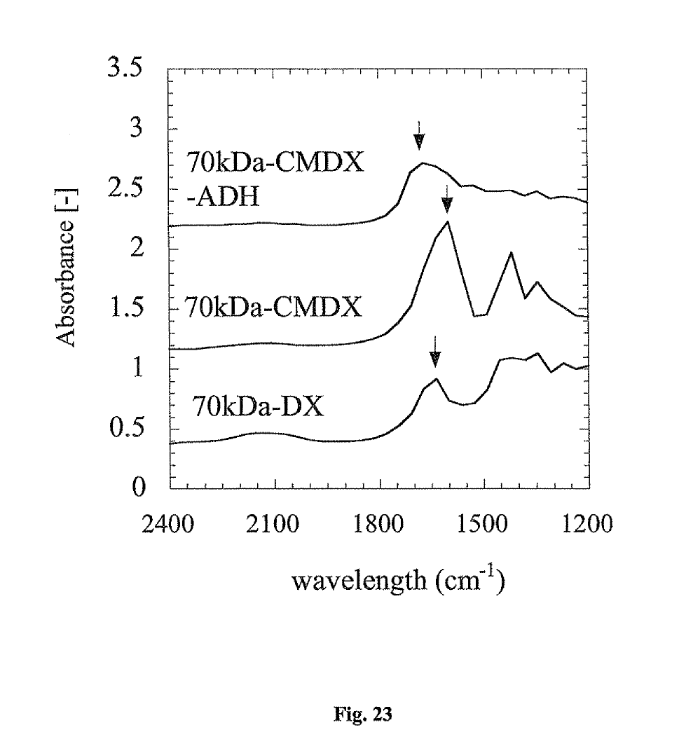

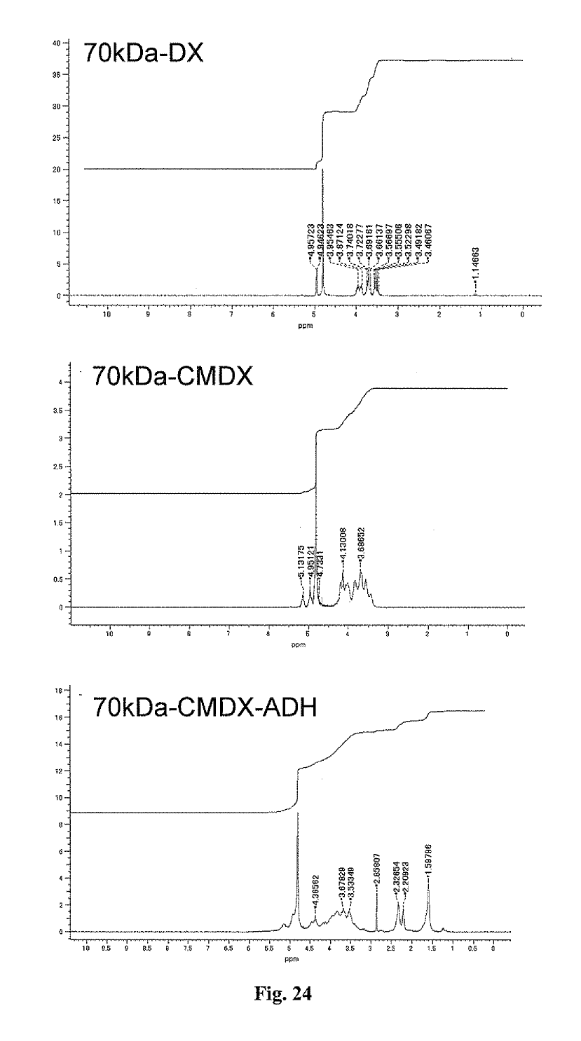

Syntheses of CMDX, CMDX-ADH were confirmed by FT-IR (FIG. 23) and ¹H NMR (FIG. 24). The results were the same for both dextrans (70 kDa and 500 kDa).

On FTIR, dextran has an absorbance peak at 1650 cm⁻¹ [Lino's paper] as shown in FIG. 23. Modification to CMDX was confirmed by demonstrating a new peak at 1608 cm⁻¹, reflecting C═O stretching by carboxyl groups. Further modification to CMDX-ADH was shown by a new peak at 1664 cm⁻¹, reflecting C═O stretching by amide groups.

Dextran does not have NMR peaks in the regions below 3.0 ppm and over 5.2 ppm. In CMDX a new peak appeared at 5.13 ppm. In CMDX-ADH new peaks appeared at 2.09 and 2.32 ppm (doublet, 4H, N—(CH₂)—C) and at 1.60 ppm (singlet, 4H, C—(CH₂)—C).

By elemental analysis of CMDX-ADH, the weight percentages of carbon, hydrogen, and nitrogen were 44.4%, 6.6%, and 9.9% in 70 kDa CMDX-ADH, and 44.7%, 6.3%, and 9.7% in 500 kDa CMDX-ADH, respectively. The degrees of modification of CMDX-ADH with adipic dihydrazide, estimated from the ratio of nitrogen to carbon, were 46% in 70 kDa CMDX-ADH and 44% in 500 kDa CMDX-ADH.

The results of FT-IR and NMR analysis of DX—CHO were identical to previously published data, confirming the synthesis. CMC—CHO was produced and characterized as reported.

In Vitro Properties of In Situ Hydrogels

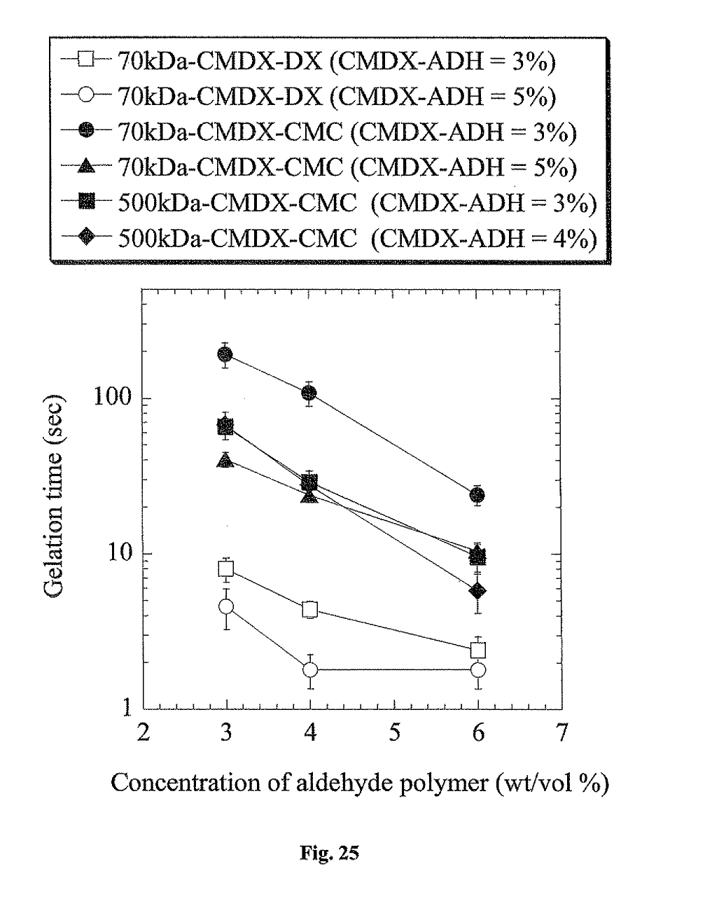

Gelation times of all hydrogels were concentration-dependent (FIG. 25), such that rapid gelation times could be obtained for all hydrogels at high concentrations of both aldehyde- and hydrazide-modified prepolymers. CMDX-DX gels were shorter than those of CMDX—CMC gels at each concentration (FIG. 25).

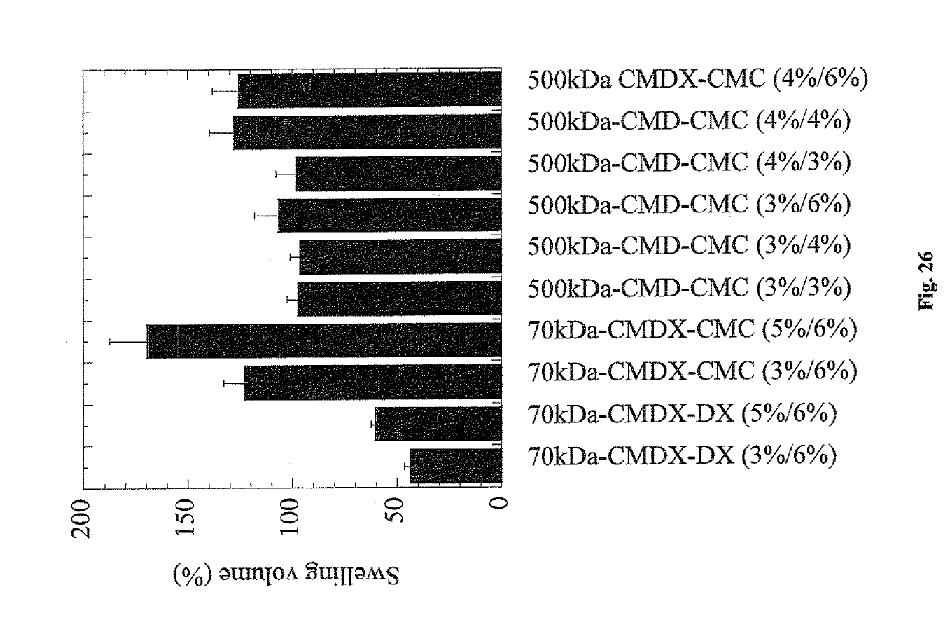

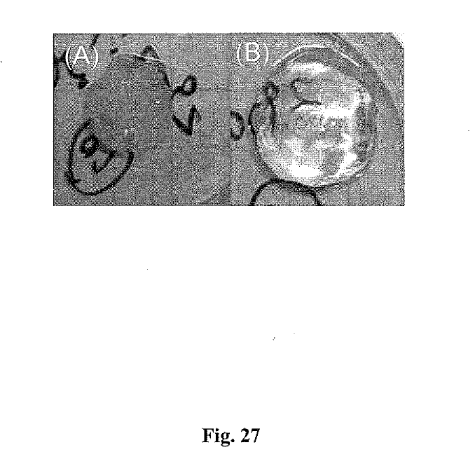

CMDX-DX gels shrank after gelation, while CMDX—CMC gels swelled after gelation, so that, for example, the swelling volume of 70 kDa-CMDX—CMC (5%/6%) was three times higher than that of CMDX-DX (5%/6%) (FIG. 26A). The physical appearance of the hydrogels quite different (FIG. 26B): CMDX-DX was yellowish and slightly opaque, while 70 kDa-CMDX—CMC was transparent and clear. The swelling of 500 kDa-CMDX—CMC (5%/6%) was intermediate between those of 70 kDa-CMDX—CMC and CMDX-DX.

Biocompatibility of the In Situ Hydrogels in Vitro Assay

Unmodified starting materials showed minial cytotoxicity in cell culture, but some modifications affected cell viability.

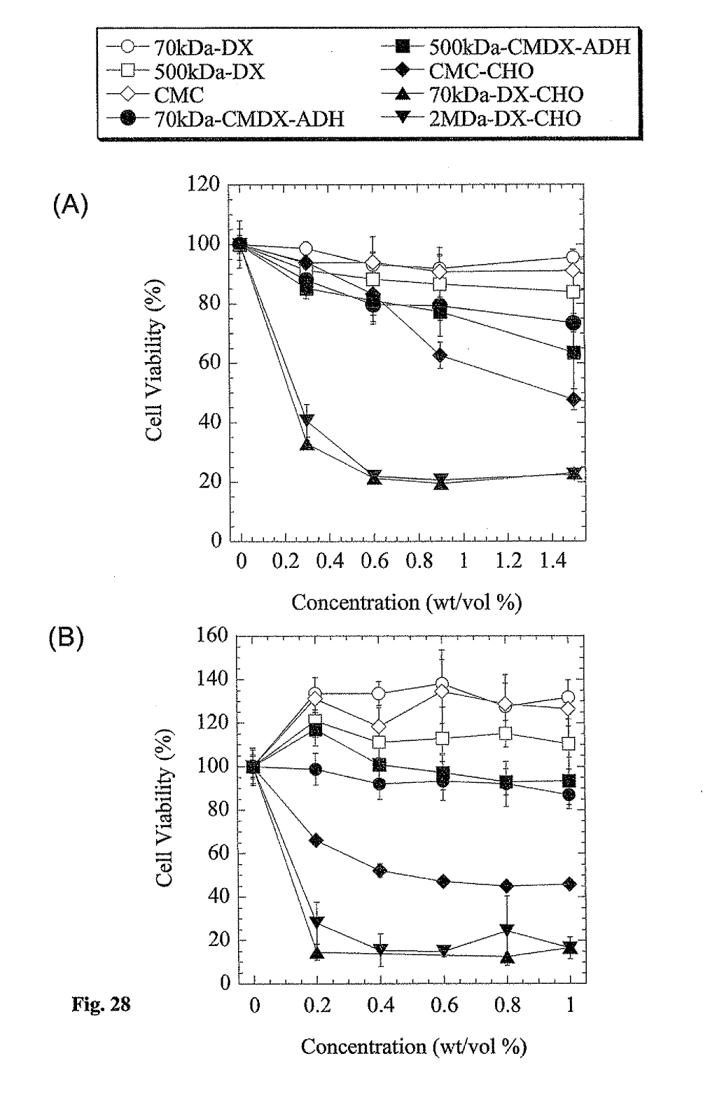

Mesothelial cells were cultured in the presence of a range of concentrations of the uncross-linked pre-polymers CMDX-ADH, DX—CHO, and CMC—CHO (FIG. 28A). CMDX-ADH and CMC—CHO showed mild dose-dependent toxicity, while DX—CHO was much more toxic.

The overall pattern of cytotoxic effects was similar in macrophages (FIG. 28B). The aldehyde derivatives of dextran were very cytotoxic,as was CMC—CHO. The 70 kDa- and 500 kDa-CMDX-ADHs had no effect on macrophage viability even at high concentrations. Unmodified CMC and DX increased cell viability at all concentrations.

From the MTT assay of both cell lines, CMDX-ADH was very biocompatible, and also its hydrogels can be expected as very biocompatible in peritoneum.

Peritoneal Adhesion-Preventing Functions of the In Situ Hydrogels

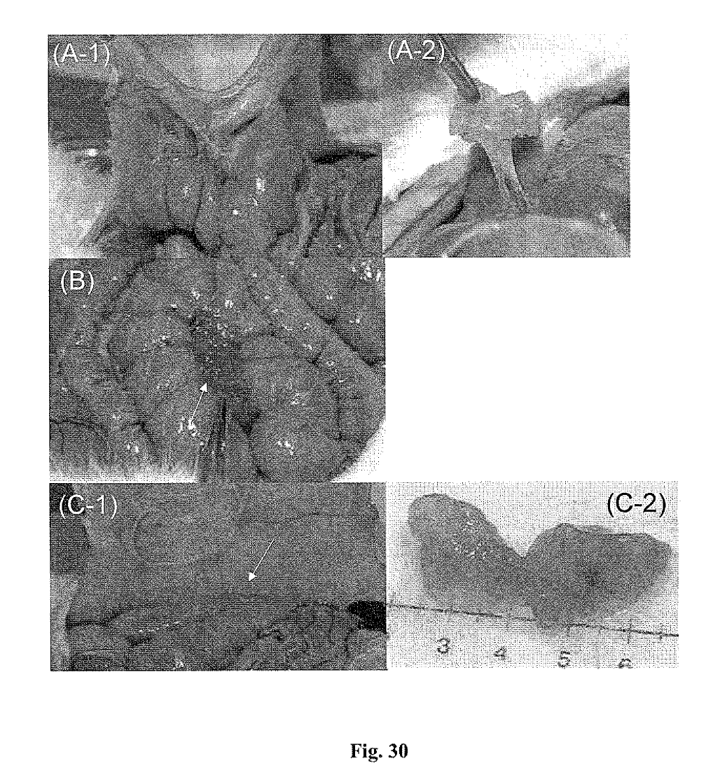

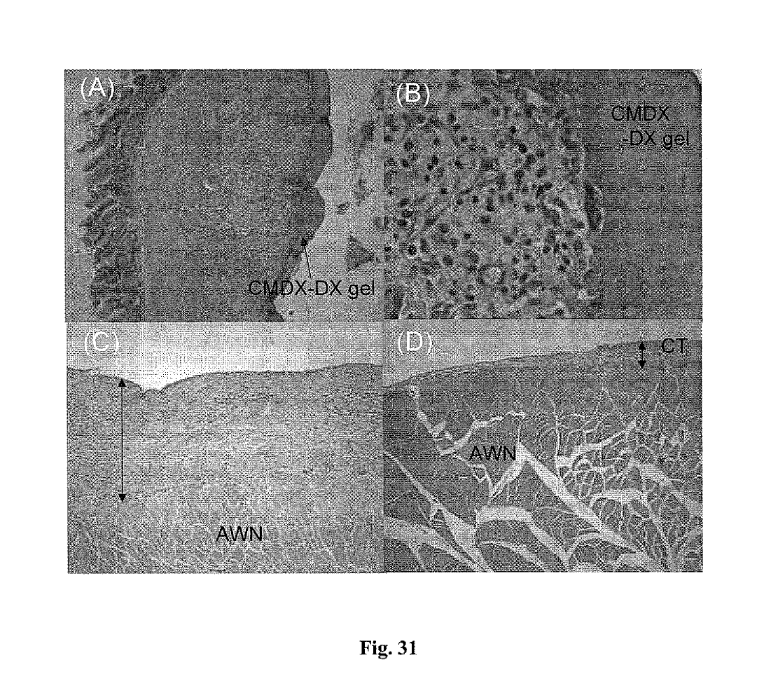

All four rabbits treated with CMDX-DX developed adhesions, and adhesion area was larger than that of the control experiment (p=0.0027; Table 13.1, FIG. 30A-1). Hydrogel debris was found as isolated clumps in the peritoneum, sometimes entrapped within the adhesions. Therefore, this gel did not function as a barrier although present at the site of injury. The gel debris was yellowish and firm, as observed in the swelling tests. It was firmly adherent to tissues (FIG. 30A-2), unlike other in situ cross-linking gels we have studied.

TABLE 13.1

| | 70 kDa- | 70 kDa- | 500 kDa- | |
|---|---|---|---|---|
| | CMDX - | CMDX - | CMDX - | Control |
| | DX | CMC | CMC | (Saline) |
| % Weight change | −6.5 ± 3.6 | −2.8 ± 2.6 | −8.4 ± 3.3 | −11.7 ± 2.4 |
| Score 3 | 4 | 1 | 1 | 3 |
| Score 2 | 0 | 0 | 0 | 1 |
| Score 1 | 0 | 1 | 0 | 0 |
| No adhesion | 0 | 2 | 3 | 0 |
| Median adhesion score | 3 | 0.5 | 0 | 3 |
| Adhesion area (cm²) | 18.3 ± 0.9 | 0.8 ± 1.5 | 0.0 ± 0.1 | 13.1 ± 1.9 |
| Material residue | 4/4 | 4/4 | 4/4 | — |

In contrast, both 70 kDa- and 500 kDa-CMDX—CMC were found distributed throughout the peritoneum, but sometimes remained in part as a contiguous mass as shown in FIG. 9B. The reddish tinge of the material is due to contamination with blood (FIG. 30C-2). Both hydrogels caused a drastic reduction in the area of adhesion formation (Table 1, FIG. 30B, C-1) compared to controls (p<0.0001 for both 70 kDa- and 500 kDa-CMDX—CMC).

The median adhesions scores for both CMDX—CMC groups were much smaller than those of either the untreated control or the CMDX-DX gels, but the sample sizes were too small to show statistical significance. Comparison of pooled data from the two CMDX—CMC groups yielded at p-value of 0.009. The median adhesion scores of both CMDX—CMC groups were similar and there was no statistically significant difference.

On histology, CMDX-DX gels were highly adherent to normal and injured cecum surface (FIG. 30A), with marked infiltration of inflammatory cells into the subjacent connective tissue (FIG. 30B). In contrast, the mesothelium recovered in animals treated with CMDX—CMC (FIG. 30C), although with a greatly thickened subjacent layer of connective tissue (FIG. 30D).

Intraperitoneal Injection of CMDX-DX Gel

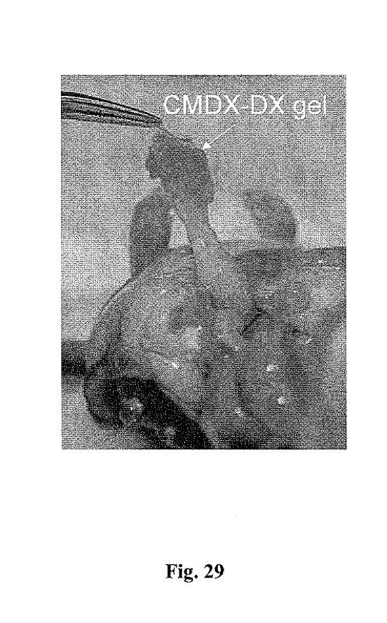

The inability of the CMDX-DX to prevent adhesions could either be due to a lack of efficacy in preventing adhesions, or due to a direct effect in causing them. To assess whether the material itself was harmful, four mice were given intraperitneal injections with CMDX-ADH and DH—CHO through a double-barreled syringe, forming CMDX-DX in situ. Two weeks after injection, animals were sacrificed and their abdominal cavities examines. CMDX-DX did not cause peritoneal adhesions, but was found in firm yellowish clumps that were firmly adherent to tissues (FIG. 29).

Discussion

Dextran is a very biocompatible and low-cost material suitable for peritoneal applications. Here we synthesized two dextran-based hydrogels that form without the need for a low-molecular weight crosslinker. Although both shared the CMDX-ADH moiety, they had strikingly different properties.

The gelation time of CMDX-DX much shorter than that of CMDX—CMC. There are several possible reasons for this difference, including viscosity, the degree of modification with hydrazide or aldehyde and others, but the principal reason may be the difference in aqueous solubility between dextran and CMC. Because the gelation time of 3 wt/vol % 500 kDa-CMDX—CMC gel was very slow like 65.8±5.0 sec, while that of 2 wt/vol % 490 kDa-HA (hyaluronic acid)-CMC gel was very quick like 18.5±1.7 sec in our previous study. The modification degrees and molecular weight of HA-ADH and CMDX-ADH were almost equal, thus the solubility of DX and CMC may be extremely poor. On the other hand, the gelation time of CMDX-DX gel was very quick, because both CMDX-ADH and DX—CHO were synthesized from the same DX. The polymers which have the same polymer backbone can mix easily each other.

CMDX-DX shrank while CMDX—CMC welled after gelation. This could be explained by electrostatic repulsion between the negative charges of CMC—CHO, while DX—CHO has no charge.

The two materials differed dramatically in their performance in preventing peritoneal adhesions. CMDX-DX made adhesions worse, while CMDX—CMC prevented them. It is possible that the cytotoxicity of DX—CHO as shown in vitro contributed to its lack of efficacy in preventing adhesions. However, that toxicity occurred over 2 (in the case of macrophages) or 3 (for mesothelial cells) days' exposure. It does not follow, that there would be sufficiently high levels of free CMDX-DX to cause tissue injury after cross-linking, which took seconds. The cross-linked material itself may be harmful.

Although considerable success has been achieved by in situ cross-linking hydrogels used as barrier devices, their degradation times can be relatively brief, especially those based on hyaluronic acid. However, the length of time for which it is necessary for barrier devices to stay in place to avoid adhesion formation is not known. In that context, CMDX-DX may prove advantageous in that there was a large amount of residual material found on necropsy, suggesting slow degradation kinetics. This slow degradation may be due to the relatively high polymer concentration, the relatively low swelling volume, and the fact that dextranase, which degrades dextran, is basically only located in liver.

Conclusion

The cross-linked hydrogel of hydrazide-modified carboxymethyldextran and aldehyde-modified carboxymethylcellulose showed efficacy in preventing peritoneal adhesions. The slow degradation rate and low cost of these gels suggests a possible role in the prevention of peritoneal adhesions.

### Example 14

### Anti-Inflammatory Activity of an In Situ Cross-Linkable Conjugate Hydrogel of Hyaluronic Acid and Dexamethasone

Introduction

Postoperative peritoneal adhesions can cause pain, bowel obstruction and infertility (DiZerega, G. S. Peritoneal Surgery, Springer, New York, 1999, which is incorporated herein by reference). A variety of polysaccharide-based hydrogel barrier systems have been used to prevent adhesions, with varying degrees of success (DiZerega, G. S. Peritoneal Surgery, Springer, New York, 1999, which is incorporated herein by reference). Hydrogels that form by cross-linking in situ are potentially useful as barriers (Johns, D. B., Rodgers, K. E., Donahue, W. D., Kiorpes, T. C., and diZerega, G. S. Reduction of adhesion formation by postoperative administration of ionically cross-linked hyaluronic acid. Fertility And Sterility 68 (1997) 37-42, which is incorporated herein by reference), (Li, H., Liu, Y. C., Shu, X. Z., Gray, S. D., and Prestwich, G. D. Synthesis and biological evaluation of a cross-linked hyaluronan-mitomycin C hydrogel. Biomacromolecules 5 (2004) 895-902, which is incorporated herein by reference), (Liu, Y. C., Li, H., Shu, X. Z., Gray, S. D., and Prestwich, G. D. Crosslinked hyaluronan hydrogels containing mitomycin C reduce postoperative abdominal adhesions. Fertility And Sterility 83 (2005) 1275-1283, which is incorporated herein by reference), Oh, S. H., Kim, J. K., Song, K. S., Noh, S. M., Ghil, S. H., Yuk, S. H., and Lee, J. H. Prevention of postsurgical tissue adhesion by anti-inflammatory drug-loaded pluronic mixtures with sol-gel transition behavior. J Biomed Mater Res A 72 (2005) 306-16, which is incorporated herein by reference), (Yeo, Y., Highley, C., Bellas, E., Ito, T., Marini, R., Langer, R., and Kohane, D. In situ cross-linkable hyaluronic acid hydrogels prevent post-operative abdominal adhesions in a rabbit model. Biomaterials 27 (2006) 4698-4705, which is incorporated herein by reference), and can be easier to apply than preformed sheets. In particular, hyaluronic acid (HA) derivatives are frequently used in the peritoneum. In situ chemically-modified HA hydrogels with or without added drugs have been used for this purpose (Johns, D. B., Rodgers, K. E., Donahue, W. D., Kiorpes, T. C., and diZerega, G. S. Reduction of adhesion formation by postoperative administration of ionically cross-linked hyaluronic acid. Fertility And Sterility 68 (1997) 37-42, which is incorporated herein by reference), (Li, H., Liu, Y. C., Shu, X. Z., Gray, S. D., and Prestwich, G. D. Synthesis and biological evaluation of a cross-linked hyaluronan-mitomycin C hydrogel. Biomacromolecules 5 (2004) 895-902, which is incorporated herein by reference), (Liu, Y. C., Li, H., Shu, X. Z., Gray, S. D., and Prestwich, G. D. Crosslinked hyaluronan hydrogels containing mitomycin C reduce postoperative abdominal adhesions. Fertility And Sterility 83 (2005) 1275-1283, which is incorporated herein by reference), (Yeo, Y., Highley, C., Bellas, E., Ito, T., Marini, R., Langer, R., and Kohane, D. In situ cross-linkable hyaluronic acid hydrogels prevent post-operative abdominal adhesions in a rabbit model. Biomaterials 27 (2006) 4698-4705, which is incorporated herein by reference). Recently, we have shown that an in situ cross-linkable hydrogel composed of hydrazone-cross-linked aldehyde- and hydrazide-modified HAs (Bulpitt, P., and Aeschlimann, D. New strategy for chemical modification of hyaluronic acid: Preparation of functionalized derivatives and their use in the formation of novel biocompatible hydrogels. Journal Of Biomedical Materials Research 47 (1999) 152-169, which is incorporated herein by reference), (Jia, X. Q., Colombo, G., Padera, R., Langer, R., and Kohane, D. S. Prolongation of sciatic nerve blockade by in situ cross-linked hyaluronic acid. Biomaterials 25 (2004) 4797-4804, which is incorporated herein by reference), was effective in preventing peritoneal adhesions in a rabbit side wall defect-cecal abrasion model (Yeo, Y., Highley, C., Bellas, E., Ito, T., Marini, R., Langer, R., and Kohane, D. In situ cross-linkable hyaluronic acid hydrogels prevent post-operative abdominal adhesions in a rabbit model. Biomaterials 27 (2006) 4698-4705, which is incorporated herein by reference).

The majority of these devices exploit the ability of the device to act as a biocompatible barrier, separating the injured surfaces during healing. Here, we have modified the hydrogel to address directly the pathophysiology of adhesion formation. Inflammation is believed to contribute to the formation of peritoneal adhesions. Quite apart from the potentially tissue-destructive effects of inflammation per se, inflammatory cells release cytokines such as tumor necrosis factor-alpha (TNF-α), interleukin-1 and -6 (IL-1 and -6). These cytokines induce the production of plasminogen activator inhibitors-1 and 2 (PAI-1 and PAI-2) from mesothelial cells, which reduces the activity of plasminogen activators (PAs), slowing the degradation of fibrin (Whawell, S. A., Scottcoombes, D. M., Vipond, M. N., Tebbutt, S. J., and Thompson, J. N. Tumor Necrosis Factor-Mediated Release Of Plasminogen-Activator Inhibitor-1 By Human Peritoneal Mesothelial Cells. British Journal Of Surgery 81 (1994) 214-216, which is incorporated herein by reference), (Whawell, S. A., and Thompson, J. N. Cytokine-Induced Release Of Plasminogen-Activator Inhibitor-1 By Human Mesothelial Cells. European Journal Of Surgery 161 (1995) 315-318, which is incorporated herein by reference), (Vanhinsbergh, V. W. M., Bauer, K. A., Kooistra, T., Kluft, C., Dooijewaard, G., Sherman, M. L., and Nieuwenhuizen, W. Progress Of Fibrinolysis During Tumor-Necrosis-Factor Infusions In Humans—Concomitant Increase In Tissue-Type Plasminogen-Activator, Plasminogen-Activator Inhibitor Type-1, And Fibrin(Ogen) Degradation Products. Blood 76 (1990) 2284-2289, which is incorporated herein by reference), (Mullarky, I. K., Szaba, F. M., Berggren, K. N., Kummer, L. W., Wilhelm, L. B., Parent, M. A., Johnson, L. L., and Smiley, S. T. Tumor necrosis factor alpha and gamma interferon, but not hemorrhage or pathogen burden, dictate levels of protective fibrin deposition during infection. Infection And Immunity 74 (2006) 1181-1188, which is incorporated herein by reference) Mullarky, I. K., Szaba, F. M., Berggren, K. N., Kummer, L. W., Wilhelm, L. B., Parent, M. A., Johnson, L. L., and Smiley, S. T. Tumor necrosis factor alpha and gamma interferon, but not hemorrhage or pathogen burden, dictate levels of protective fibrin deposition during infection. Infection And Immunity 74 (2006) 1181-1188. These processes can promote adhesion formation. Mitigation of pro-inflammatory cytokine release could enhance the prevention of peritoneal adhesions. For this reason, several investigators have tested the effectiveness of anti-inflammatory drugs against peritoneal adhesions, including nonsteroidal anti-inflammatory drugs (NSAIDs) such as ibuprofen (Oh, S. H., Kim, J. K., Song, K. S., Noh, S. M., Ghil, S. H., Yuk, S. H., and Lee, J. H. Prevention of postsurgical tissue adhesion by anti-inflammatory drug-loaded pluronic mixtures with sol-gel transition behavior. J Biomed Mater Res A 72 (2005) 306-16, which is incorporated herein by reference), (Bateman, B. G., Nunley, W. C., Jr., and Kitchin, J. D., 3rd. Prevention of postoperative peritoneal adhesions with ibuprofen. Fertil Steril 38 (1982) 107-8, which is incorporated herein by reference), (Nishimura, K., Nakamura, R. M., and diZerega, G. S. Biochemical evaluation of postsurgical wound repair: prevention of intraperitoneal adhesion formation with ibuprofen. J Surg Res 34 (1983) 219-26, which is incorporated herein by reference), (Rodgers, K., Girgis, W., diZerega, G. S., Bracken, K., and Richer, L. Inhibition of postsurgical adhesions by liposomes containing nonsteroidal antiinflammatory drugs. Int J Fertil 35 (1990) 315-20, which is incorporated herein by reference), (LeGrand, E. K., Rodgers, K. E., Girgis, W., Campeau, J. D., and Dizerega, G. S. Comparative efficacy of nonsteroidal anti-inflammatory drugs and anti-thromboxane agents in a rabbit adhesion-prevention model. J Invest Surg 8 (1995) 187-94, which is incorporated herein by reference), (Lee, J. H., Go, A. K., Oh, S. H., Lee, K. E., and Yuk, S. H. Tissue anti-adhesion potential of ibuprofen-loaded PLLA-PEG diblock copolymer films. Biomaterials 26 (2005) 671-8, which is incorporated herein by reference) and glucocorticoids such as dexamethasone (Hockel, M., Ott, S., Siemann, U., and Kissel, T. Prevention Of Peritoneal Adhesions In The Rat With Sustained Intraperitoneal Dexamethasone Delivered By A Novel Therapeutic System. Annales Chirurgiae Et Gynaecologiae 76 (1987) 306-313, which is incorporated herein by reference), (Buckenmaier, C. C., Pusateri, A. E., Harris, R. A., and Hetz, S. P. Comparison of antiadhesive treatments using an objective rat model. American Surgeon 65 (1999) 274-282, which is incorporated herein by reference), (Kucukozkan, T., Ersoy, B., Uygur, D., and Gundogdu, C. Prevention of adhesions by sodium chromoglycate, dexamethasone, saline and aprotinin after pelvic surgery. ANZ J Surg 74 (2004) 1111-5, which is incorporated herein as reference). These have been incorporated into matrix materials such as liposomes (Rodgers, K., Girgis, W., diZerega, G. S., Bracken, K., and Richer, L. Inhibition of postsurgical adhesions by liposomes containing nonsteroidal antiinflammatory drugs. Int J Fertil 35 (1990) 315-20, which is incorporated herein by reference), poly(L-lactic acid) (PLLA)-polyethylene glycol (PEG) diblock copolymers films (Lee, J. H., Go, A. K., Oh, S. H., Lee, K. E., and Yuk, S. H. Tissue anti-adhesion potential of ibuprofen-loaded PLLA-PEG diblock copolymer films. Biomaterials 26 (2005) 671-8, which is incorporated herein by reference), a mixture of poloxamer and alginate hydrogels (Oh, S. H., Kim, J. K., Song, K. S., Noh, S. M., Ghil, S. H., Yuk, S. H., and Lee, J. H. Prevention of postsurgical tissue adhesion by anti-inflammatory drug-loaded pluronic mixtures with sol-gel transition behavior. J Biomed Mater Res A 72 (2005) 306-16, which is incorporated herein by reference), and poly(lactide-co-glycolide) (PLGA) microparticles (Hockel, M., Ott, S., Siemann, U., and Kissel, T. Prevention Of Peritoneal Adhesions In The Rat With Sustained Intraperitoneal Dexamethasone Delivered By A Novel Therapeutic System. Annales Chirurgiae Et Gynaecologiae 76 (1987) 306-313, which is incorporated herein by reference). We have found that hydrogel-based systems are generally more biocompatible in the peritoneum than hydrophobic polymeric devices (e.g. those composed of PLGA) (Yeo, Y., Highley, C., Bellas, E., Ito, T., Marini, R., Langer, R., and Kohane, D. In situ cross-linkable hyaluronic acid hydrogels prevent post-operative abdominal adhesions in a rabbit model. Biomaterials 27 (2006) 4698-4705, which is incorporated herein by reference), (Kohane, D. S., Tse, J. Y., Yeo, Y., Padera, R., Shubina, M., and Langer, R. Biodegradable polymeric microspheres and nanospheres for drug delivery in the peritoneum. Journal Of Biomedical Materials Research Part A 77A (2006) 351-361, which is incorporated herein by reference), but have much more rapid release kinetics because low molecular weight drugs diffuse rapidly through the hydrogel matrix. One approach to controlling this problem is to conjugate the small molecule to the hydrogel (McLeod, A. D., Tolentino, L., and Tozer, T. N. Glucocorticoid-Dextran Conjugates As Potential Prodrugs For Colon-Specific Delivery—Steady-State Pharmacokinetics In The Rat. Biopharmaceutics & Drug Disposition 15 (1994) 151-161, which is incorporated herein by reference), (McLeod, A. D., Friend, D. R., and Tozer, T. N. Glucocorticoid-Dextran Conjugates As Potential Prodrugs For Colon-Specific Delivery—Hydrolysis In Rat Gastrointestinal-Tract Contents. Journal Of Pharmaceutical Sciences 83 (1994) 1284-1288, which is incorporated herein by reference), (Zhou, S. Y., Mei, Q. B., Liu, L., Guo, X., Qiu, B. S., Zhao, D. H., and Cho, C. H. Delivery of glucocorticoid conjugate in rat gastrointestinal tract and its treatment for ulcerative colitis. Acta Pharmacologica Sinica 22 (2001) 761-764, which is incorporated herein by reference), (Pang, Y. N., Zhang, Y., and Zhang, Z. R. Synthesis of an enzyme-dependent prodrug and evaluation of its potential for colon targeting. World Journal Of Gastroenterology 8 (2002) 913-917, which is incorporated herein by reference), (Pouyani, T., and Prestwich, G. D. Functionalized Derivatives of Hyaluronic-Acid Oligosaccharides—Drug Carriers and Novel Biomaterials. Bioconjugate Chemistry 5 (1994) 339-347, which is incorporated herein by reference), (Prestwich et al. Controlled chemical modification of hyaluronic acid: synthesis, applications, and biodegradation of hydrazide derivatives. Journal Of Controlled Release 53 (1998) 93-103, which is incorporated herein by reference), (Rajewski et al. Enzymatic And Nonenzymatic Hydrolysis Of A Polymeric Prodrug—Hydrocortisone Esters Of Hyaluronic-Acid. International Journal Of Pharmaceutics 82 (1992) 205-213, which is incorporated herein by reference), (Everts et al. Selective intracellular delivery of dexamethasone into activated endothelial cells using an E-selectin-directed immunoconjugate. Journal Of Immunology 168 (2002) 883-889, which is incorporated herein by reference), (Melgert et al. Targeting dexamethasone to Kupffer cells: Effects on liver inflammation and fibrosis in rats. Hepatology 34 (2001) 719-728, which is incorporated herein by reference). These conjugates can control the release kinetics, but the conjugate polymers themselves can be cleared rapidly from the peritoneum.

To address the problems of anti-inflammatory drug release from a hydrogel matrix and hydrogel diffusion out of the peritoneum, we designed and synthesized a hydrogel that combined in situ cross-linking properties with drug conjugation (Pouyani, T., and Prestwich, G. D. Functionalized Derivatives of Hyaluronic-Acid Oligosaccharides—Drug Carriers and Novel Biomaterials. Bioconjugate Chemistry 5 (1994) 339-347, which is incorporated herein by reference). We produced HAs that were conjugated via an ester linkage to the potent synthetic glucocorticoid agonist dexamethasone and which were also modified with a hydrazide or aldehyde group so that they were cross-linked to other HAs by hydrazone bonds. Here we characterize these materials and demonstrate their anti-inflammatory activity in vitro and in vivo.

Materials and Methods

Materials

Dexamethasone, succinic anhydride, ethanol, 1-[3-(dimethylamino)propyl]-3-ethylcarbodiimide hydrochloride (EDC), 4-dimethylaminopyridine, N-hydroxysuccinimide (NHS), dicyclohexylcarbodiimide (DCC), anhydrous acetone, dimethyl sulfoxide (DMSO), adipic dihydrazide (ADH), hydroxybenzotriazole (HOBt), sodium periodate, ethylene glycol, tert-buthyl carbazate, sodium chloride, phosphoric acid, and sodium were purchased from Aldrich. HA (Mw=490 kDa or 1.36 MkDa) was purchased from Genzyme.

Methods

Synthesis of hyaluronic acid-adipic dihydrazide (HA-ADH) and hyaluronic acid-aldehyde (HA-ALD)

Syntheses were performed as described (Yeo et al. In situ cross-linkable hyaluronic acid hydrogels prevent post-operative abdominal adhesions in a rabbit model. Biomaterials 27 (2006) 4698-4705, which is incorporated herein by reference), (Bulpitt et al. New strategy for chemical modification of hyaluronic acid: Preparation of functionalized derivatives and their use in the formation of novel biocompatible hydrogels. Journal Of Biomedical Materials Research 47 (1999) 152-169, which is incorporated herein by reference), (Jia, X. Q., Colombo, G., Padera, R., Langer, R., and Kohane, D. S. Prolongation of sciatic nerve blockade by in situ cross-linked hyaluronic acid. Biomaterials 25 (2004) 4797-4804, which is incorporated herein by reference). HA-ADH and HA-ALD were synthesized from 1.36 MDa HA and 490 kDa HA, respectively.

Synthesis of dexamethasone-succinate (dex-suc).

This procedure was based on previous reports (Pang, Y. N., Zhang, Y., and Zhang, Z. R. Synthesis of an enzyme-dependent prodrug and evaluation of its potential for colon targeting. World Journal Of Gastroenterology 8 (2002) 913-917, which is incorporated herein by reference), (Everts, M., Kok, R. J., Asgeirsdottir, S. A., Melgert, B. N., Moolenaar, T. J. M., Koning, G. A., van Luyn, M. J. A., Meijer, D. K. F., and Molema, G. Selective intracellular delivery of dexamethasone into activated endothelial cells using an E-selectin-directed immunoconjugate. Journal Of Immunology 168 (2002) 883-889, which is incorporated herein by reference). 2.455 g dexamethasone (6.25 mmol), 10.52 g succinic anhydride and 0.795 g 4-dimethylaminopyridine were dissolved in 400 ml anhydrous acetone under nitrogen gas. The solution was stirred at room temperature overnight. After the evaporation of acetone, the white crystal was dissolved in 36 ml ethanol, then 84 ml pure water was gradually added. The solution was kept at 4° C. for 2 days, and white needle-shaped crystal precipitated. It was filtered and dried under reduced pressure. This reprecipitation was performed twice (yield: 90-95%).

### Synthesis of N-hydroxysuccinimide dexamethasone-succinate (NHS-dex-suc)

This procedure was based on a previous report (Pouyani, T., and Prestwich, G. D. Functionalized Derivatives of Hyaluronic-Acid Oligosaccharides—Drug Carriers and Novel Biomaterials. Bioconjugate Chemistry 5 (1994) 339-347, which is incorporated herein by reference). 1.3930 g dex-suc, 0.3355 g N-hydroxysuccinimide (NHS), and 0.6040 mg dicyclohexylcarbodiimide (DCC) were dissolved in 80 ml acetone, and stirred for 16 h at room temperature, producing a white crystalline precipitate. The crystals were recovered by filtration, acetone was removed by evaporation and dry white crystals were obtained and used without further purification (yield: 90-95%).

Synthesis of hyaluronic acid-adipic dihydrazide-dexamethasone-succinate (HA-DEX).

This procedure was based on a previous report (Pouyani, T., and Prestwich, G. D. Functionalized Derivatives of Hyaluronic-Acid Oligosaccharides—Drug Carriers and Novel Biomaterials. Bioconjugate Chemistry 5 (1994) 339-347, which is incorporated herein by reference). 100 mg HA-ADH was dissolved in 13.33 ml NaHCO₃ buffer (pH=8.5). 125.8 mg of NHS-dex-suc was dissolved in 26.67 ml DMF (dimethylformamide). The NHS-dex-suc solution was poured into the HA-ADH solution over 30 min and stirred at room temperature for 18 h. The polymer was reprecipitated in 300 ml acetone, then dialyzed against water for 3 days. The purified product was lyophilized then stored at 4° C. (yield: 70-80%).

Preparation of Disc Hydrogels (HAX and HAX-DEX)

Disc-shaped hydrogels of HA-DEX cross-linked to HA-ALD (HAX-DEX) were prepared. The 2% (w/v) aqueous solutions of HA-DEX and HA-ALD were injected into a rubber mold sandwiched between two slide glasses using a double syringe (Baxter: Deerfield, Ill.). The diameter and the thickness of the prepared hydrogel were 1.2 cm and 3.5 mm, respectively. Disc-shaped hydrogels of HA-ADH cross-linked to HA-ALD (HAX) were prepared in the same manner.

Characterization of the Polymers

Dexamethasone and dex-succinate were dissolved in d₆-DMSO, and HA-ADH and HA-DEX were dissolved in D₂O, and analyzed by ¹H-NMR spectroscopy (Varian Unity 300 spectrophotometer).

The synthesis and purity of dex-suc, NHS-dex-suc, HA-ADH, and HA-DEX were verified by high performance liquid chromatography (Agilent technologies Series 1100), using an Atlantis dC18 analytical column (dC18; 4.6×250 mm; particle size 5 μm). The mobile phase was a mixture of acetonitrile and NaH₂PO₄/H₃PO₄ buffer (60/40; pH 3.8). The flow rate was 1 ml/min, and UV absorbance was measured at 246 nm (Hewlett Packard: G1314A). The amount of dexamethasone coupled to HA was analyzed by HPLC after alkaline hydrolysis of the succinate linker as reported (Everts et al. Selective intracellular delivery of dexamethasone into activated endothelial cells using an E-selectin-directed immunoconjugate. Journal Of Immunology 168 (2002) 883-889, which is incorporated herein by reference).

Characterization of the Hydrogels.

Gelation time was measured as we have reported (Yeo, Y., Highley, C., Bellas, E., Ito, T., Marini, R., Langer, R., and Kohane, D. In situ cross-linkable hyaluronic acid hydrogels prevent post-operative abdominal adhesions in a rabbit model. Biomaterials 27 (2006) 4698-4705, which is incorporated herein by reference). In brief, 0.1 ml of 2% (w/v) HA-ALD in aqueous solution was added to 0.1 ml 2% (w/v) aqueous HA-DEX or HA-ADH solution with stirring using a magnetic bar, and the time until the mixture became a globule of hydrogel was measured.

The shear modulus of the gel discs were measured by a rheometer (TA Instruments: AR1000, New Castle, Del.). The hydrogels discs were prepared as above then allowed to swell for 5 days in PBS buffer (pH=7.4), and the swelling volume was measured gravimetrically. Creep and relaxation tests were performed at different shear stresses. The shear modulus, G, was calculated from the slope of the linear relationship between stress and strain. The R² values of fitted lines between stress and strain were above 0.95.

Viability Assay.

In vitro cell viability in the presence of HA, HA-ADH, HA-ALD, and HA-DEX was determined using the MTT assay (Promega) using a human mesothelial cell line (CRL-9444: ATCC). Cells were grown and maintained in complete growth medium (Medium199 with Earle's BSS, 0.75 mM L-glutamine and 1.25 g/L sodium bicarbonate supplemented with with 3.3 nM epidermal growth factor, 400 nM hydrocortisone, 870 nM insulin, 20 mM HEPES and 10% fetal bovine serum) at 37° C. in 5% CO₂. 5×10⁴ cells were put into each well of a 24-well plate, and incubated at 37° C. in 5% CO₂ overnight. The medium was replaced with media containing different concentration of HA, HA-ADH, HA-DEX or HA-ALD. On the third day after adding those materials, the MTT assay was performed. 100 μl of tetrazolium salt solution was added into each well and incubated at 37° C. for 4 h. The purple formazan produced by active mitochondria was solubilized using 1 ml detergent solution and then measured at 570 nm by a plate reader (Molecular Devices SpectraMax 384). The absorbance values were normalized to wells where no test materials were added to the media.

Kinetics of Dexamethasone Release and HAX-DEX Degradation.

Disc-shaped HAX-DEX hydrogels were prepared as above using 2% (w/v) gel precursor solutions. The disc-shaped hydrogels were immersed in 4 ml of DMEM (Dulbecco's Modified Eagle Medium: GIBCO, Cat #10569-010), DMEM with 0.5% BSA (bovine serum albumin: Aldrich), or DMEM with 10% FBS (fetal bovine serum: GIBCO, Cat #10082-147), and incubated at 37° C. for 8 days. The media were completely replaced with fresh media on the 1st, 2nd, 3rd, and 5th days and stored at −80° C. before adding to cells.

The time course of degradation of the HAX and HAX-DEX discs was measured gravimetrically. The weight of the hydrogels, Wₛ, was measured at several time points. The swelling volume, Q (%), was calculated as Q=Wₛ/Wᵢ where Wᵢ is the initial weight of the hydrogels.

Preparation of Primary Macrophages from Mice.

C57/B16 mice were purchased from Taconic (Hudson, N.Y.). 2 ml of 3% (w/v) sterile thioglycollate solution (DIFCO Laboratories, Detroit, Mich.) was injected into the peritoneal cavity. Four days after injection, the mice were euthanized by CO₂, and 6 ml of ice-cold PBS buffer containing 5 mM EDTA was injected. After agitating the peritoneum with forceps, a macrophage-containing solution was aspirated. The cells were placed immediately into iced DMEM on ice prior to washing, counting and plating. Approximately 10⁷ cells per mouse were obtained. 5×10⁵ cells in DMEM containing 10% (v/v) FBS were added to each well of a 96-well plate, and incubated at 37° C. in 5% CO₂ overnight.

Cytokine Production by Lipopolysaccharide (LPS)-Challenged Macrophages

Non-adherent cells were removed by washing with 200 μl/well PBS buffer. Then 100 μl of DMEM with 10% FBS and 100 μl of medium pre-incunated with hydrogels (Sec. 2.2.9), or known concentrations of dexamethasone, were added to the adherent macrophages in each well. After incubation for 24 h at 37° C. in 5% CO₂, 25 μl of 900 ng/ml of LPS (Sigma, St. Louis, Mo.; Catalog #L-4391) was added to each well to a final concentration of 100 ng/ml. After incubation for a further 16 h, the media were collected and stored at −80° C. until analyzed. The concentration of TNF-α, IL-6, and dexamethasone in those media were measured by ELISA (enzyme-linked immunosorbent assay). The ELISA kits for TNF-α, IL-6, and dexamethasone were purchased from R&D Systems (DuoSet, Cat DY406), BioLegend (Mouse IL-6 ELISA MAX™ Set (Deluxe)), and Neogen Corporation, respectively.

In Vivo Experiments.

Animals were cared for in compliance with protocols approved by the Massachusetts Institute of Technology Committee on Animal Care, in conformity with the NIH guidelines for the care and use of laboratory animals (NIH publication #85-23, revised 1985). Male SV129 mice weighing 25-30 g were anesthetized with 2-3% isoflurane in oxygen. The dorsal midline was shaved and cleaned with 70% isopropanol in water. One ml of HAX-DEX was injected from a double-barreled syringe (0.5 ml of HA-DEX in one syringe, 0.5 ml of HA-ALD in the other) subcutaneously in the dorsal midline. Two days after injection, animals were sacrificed and tissues were processed for histology using standard techniques.

Statistical Analysis

Data were analyzed by Welch's t-tests (t-test with unequal variance between groups). In order to compare the medium effect in release kinetics test, ANOVAs were performed before Welch's t-tests. Paired t-tests were used in the comparison between different time points of the same media and gel. All the statistical tests were done using KaleidaGraph® (Synergy Software). A p value<0.05 was considered statistically significant.

Results

Characterization of the Polymers (HA-ADH and HA-DEX)

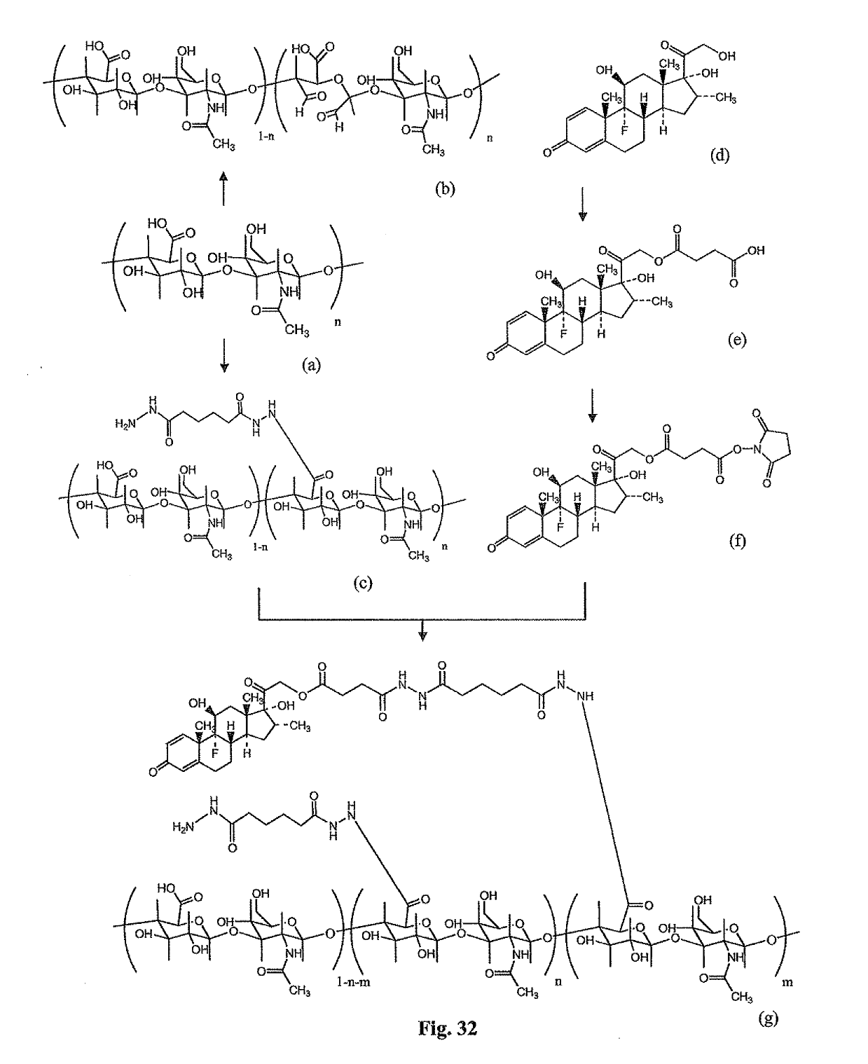

All the materials were synthesized according to the schema in FIG. 32.

The synthesis of dex-suc was confirmed by NMR spectra, by the chemical shift of 21-methylene protons of dexamethasone at 4.48 and 4.70 ppm to 4.78 and 5.03 ppm respectively. Also, the chemical shifts of 24-methylene and 25-methylene protons appeared at 2.59 ppm. The elution times of dexamethasone dex-suc, and NHD-dex-suc by HPLC were 4.9 min, 5.5, and 7.9 min respectively. By HPLC, the efficiency of the conversion of dexamethasone to dex-suc and NHS-dex-suc were >99% and 96% respectively.

The synthesis of HA-ADH was confirmed by ¹H-NMR spectra; 48.4% of D-glucuronic acid residues were modified by ADH (Yeo, Y., Highley, C., Bellas, E., Ito, T., Marini, R., Langer, R., and Kohane, D. In situ cross-linkable hyaluronic acid hydrogels prevent post-operative abdominal adhesions in a rabbit model. Biomaterials 27 (2006) 4698-4705, which is incorporated herein by reference). This was calculated from the ratio of the integrated value of the peak for the methyl proton of ADH residues at 1.62 ppm to that of the methyl proton of N-acetyl-D glucosamine residues at 2.00 ppm.

The synthesis of HA-DEX from HA-ADH was confirmed by HPLC and ¹H-NMR. HA-DEX had a UV absorbance peak at 246 nm by HPLC measurement, that HA-ADH did not. The retention time of HA-DEX was 3.0 min. The synthesis of HA-DEX from HA-ADH was also confirmed on NMR spectra, by the appearance of the chemical shift of the 24-methylene and 25-methylene proton of the succinate linker of dex-succinate at 2.63 ppm. We determined that 13.1% of D-glucuronic acid residues reacted with succinate linkers of NHS-dex-suc, from the ratio of the integrated value of the peak from the methylene proton of the succinate linker residue at 2.63 ppm to that of the methyl proton of N-acetyl-D glucosamine residues at 2.00 ppm. From this we calculated that 35.3% (=48.4%-13.1%) of ADH residues were not modified by the succinate linker, and that 27% (=13.1%/48.4%) of the HA-ADH residues reacted with NHS-dex-suc.

Pilot studies showed that this HA-dexamethasone product released a large proportion of its dexamethasone over a short period of time, which was not biologically desirable due to the high potency of the compound. Furthermore, our experience with some polymeric systems (e.g. poly(lactic-co-glycolic) acid microspheres (Kohane, D. S., Tse, J. Y., Yeo, Y., Padera, R., Shubina, M., and Langer, R. Biodegradable polymeric microspheres and nanospheres for drug delivery in the peritoneum. Journal Of Biomedical Materials Research Part A 77A (2006) 351-361, which is incorporated herein by reference) suggested that the increased hydrophobicity of this material might not be favorable for its biocompatibility for some desired uses (e.g. in the peritoneum). Consequently, we lowered the dexamethasone loading by exhaustive dialysis. On HPLC analysis of the final product, the percentage of HA-ADH coupled to dexamethasone in the final product was only 0.1-0.2% (n=3), suggesting that almost all of it was released by hydrolysis of succinate linkers during dialysis. Even this very low final degree of modification of HA with dexamethasone had an effect on the solubility of the conjugates in water, as evidenced by increased turbidity of the material in solution. It is this final product that is referred to as HA-DEX below.

Characterization of the Hydrogels (HAX and HAX-DEX).

The gelation time of HAX-DEX (22.3±0.5 sec) was longer than that of HAX (3.5±1.0; n=4 in each group, p<0.0001), presumably because the number of hydrazide groups on HA-ADH available for cross-linking was decreased by 27% by the modification with dexamethasone.

When cross-linked in PBS, both HAX and HAX-DEX swelled 206.2±14.9% and 235.6±36.9% (n=4, p=0.21) compared with their initial volumes after immersion in PBS buffers for 5 day.

The shear stress values of HAX and HAX-DEX were 32.4±17.2 Pa (n=4) and 22.1±13.5 Pa (n=4) respectively, but the difference was not statistically significant. These values indicated that both hydrogels were highly deformable, and suggest that HAX-DEX was crosslinked to approximately the same degree as HAX.

The Effect of the Synthesized Polymers on Mesothelial Cell Viability

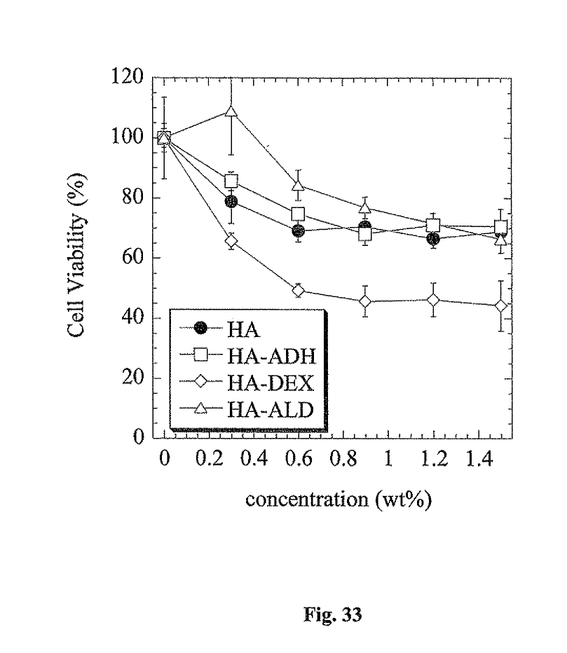

HAX and HAX-DEX gel release polymer fragments such as HA-ADH, HA-ALD or HA-DEX during their degradation. MTT assays were done to study the effect of the synthesized polymers on mesothelial cell viability. Unmodified HA, HA-ALD, and HA-ADH (n=4 for all) showed small reductions in cell viability (FIG. 33). (For unmodified HA and HA-ADH, p<0.005 compared to control at all concentrations. For HA-ALD, p=0.4, 0.07, 0.017, and 0.003 at 0.3, 0.6, 0.9, and 1.5% respectively.) HA-DEX (n=4) caused a larger reduction in the viability of mesothelial cells (e.g. comparison between HA-ADH and HA-DEX; p<0.005 at all concentrations) (FIG. 33).

Release Kinetics of Dexamethasone and Time Course of Hydrogel Volumes in Cell Culture Media.

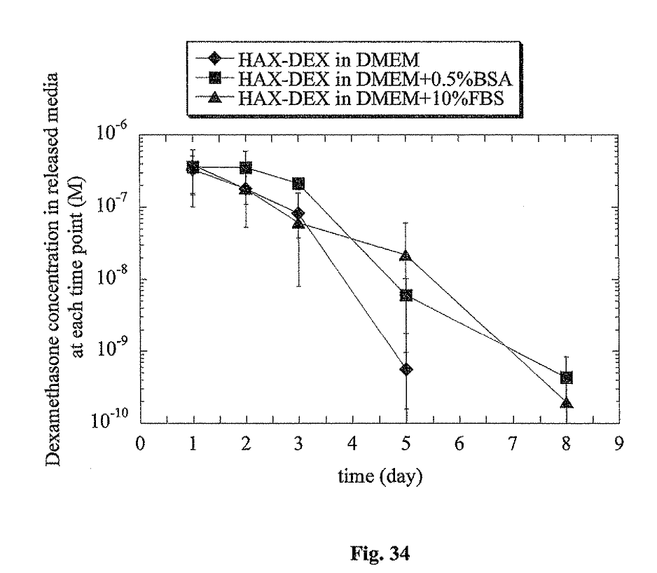

HAX-DEX gels were incubated in cell culture media (DMEM). At 1, 2, 3, 5, and 8 days, the media were changed and the concentration of dexamethasone released was measured using an ELISA as described in Methods (FIG. 34). In separate groups, we used media that were more representative of physiologically relevant fluids: 0.5% bovine serum albumin (BSA), or 10% fetal bovine serum (FBS). Release of dexamethasone occurred at a relatively constant rate for the first three days, then declined gradually in all three conditions. Total released dexamethasone was 0.94±0.20 μg in DMEM, 1.33±0.39 μg in DMEM with BSA, and 1.02±0.41 μg in DMEM with FBS, respectively, which corresponded to 17.8±3.8% of total conjugated dexamethasone released in DMEM, 25.3±7.4% in DMEM with BSA, and 19.3±7.8% in DMEM with FBS, respectively. Thus, about 20% of dexamethasone was released as the free drug by the cleavage of succinate linkers during the release experiments. At the end of those experiments, there was no gel mass left; we speculate that the remaining 80% of dexamethasone was released conjugated to polymer fragments. GPC of the release media did demonstrate the presence of macromers derivatized with dexamethasone, but it was not possible to quantitate the amount released in this manner directly. There was no statistically significant difference in dexamethasone concentration in different media at any time point except at 5th day, when dexamethasone concentration in BSA media was higher than in pure DMEM (p=0.031) and FBS media (p=0.009).

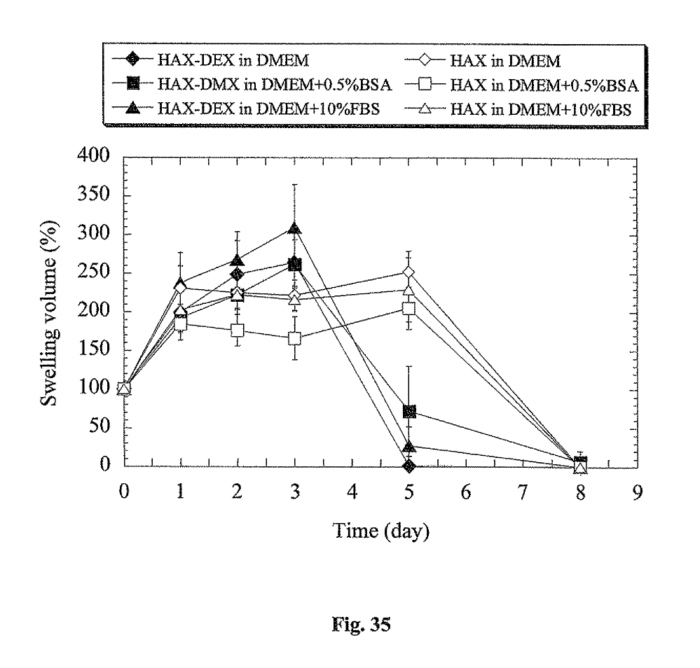

The swelling and degradation of HAX and HAX-DEX hydrogels were followed in cell culture media (FIG. 35). Both showed swelling over several days. There was no statistically significant difference in swelling between HAX and HAX-DEX on the first day, but by the third day HAX-DEX had swelled more than HAX in DMEM+BSA and DMEM+FBS (p<0.05). The degradation rate of HAX-DEX was faster than that of HAX, such that the volume of HAX-DEX gels was markedly reduced on day 5.

Anti-Inflammatory Effects of HAX-DEX.

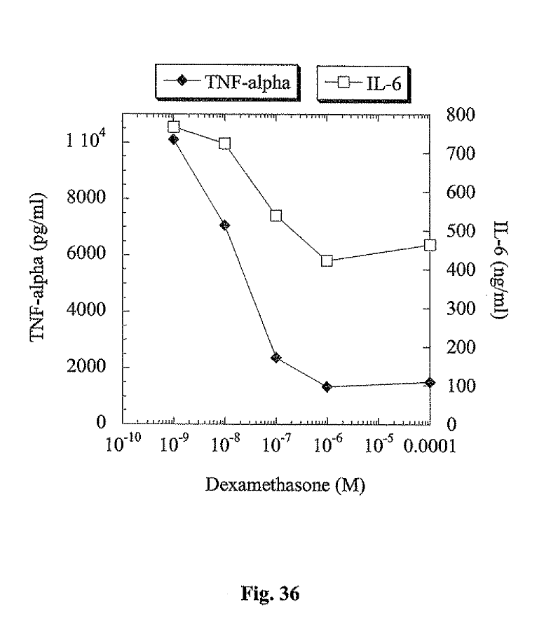

Dexamethasone produces a dose-dependent reduction in the production of IL-6 and TNF-α in macrophages stimulated with lipopolysaccharide (LPS) (FIG. 36). The concentrations of IL-6 and TNF-α in cells stimulated with LPS without dexamethasone was 773±13 ng/ml and 11723±87 pg/ml, respectively (n=4). Dexamethasone concentrations lower than 10⁻⁹ M had no effect on the production of these two molecules.

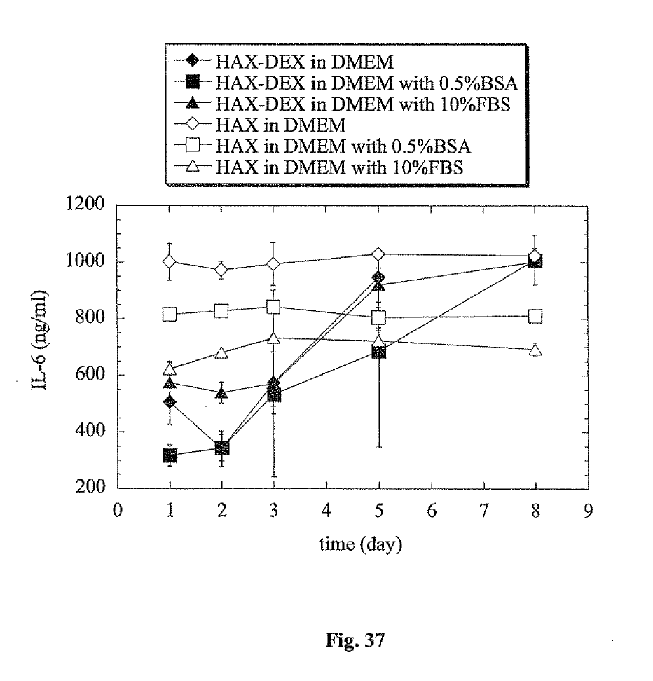

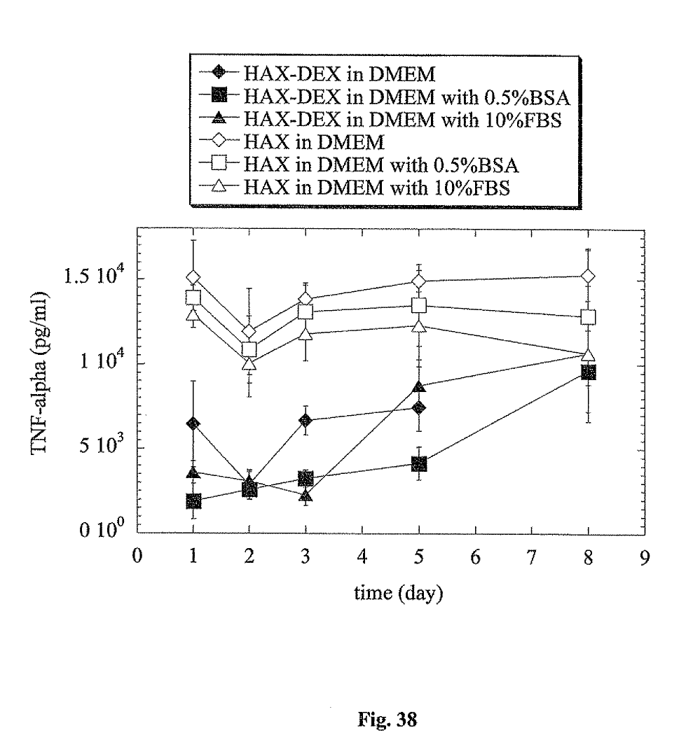

In order to investigate the anti-inflammatory effectiveness of HAX-DEX, LPS-stimulated primary peritoneal macrophages were treated with the release media generated by HAX and HAX-DEX in the experiments in the preceding section. The concentrations of IL-6 (FIG. 37) and TNF-α (FIG. 38) in those media were measured after exposure to peritoneal macrophages. Comparison of FIG. 36 to FIGS. 37 and 38 reveals that media derived from release experiments with HAX did not significantly attenuate the production of IL-6 or TNF-α. In contrast, exposure to those from HAX-DEX produced a marked reduction in both. Statistically significant suppression of the production of IL-6 (FIG. 37) and TNF-α (FIG. 38) by peritoneal macrophages was seen in HAX-DEX compared to HAX for 3 and 5 days respectively.

Biocompatibility and Tissue Reaction

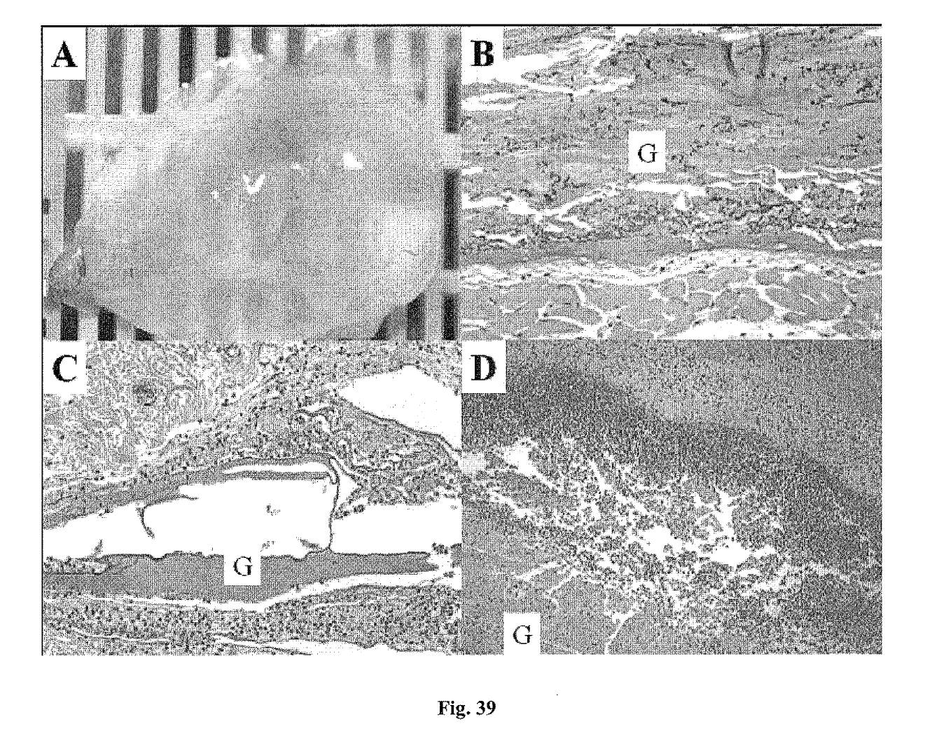

Male SV129 mice were injected with 1 ml of either HAX or HAX-DEX subcutaneously (n=4 each). Two days after injection, animals were euthanized and shaved. The contour of the pockets of HAX-DEX were more clearly demarcated than those of HAX as seen through the skin, and in subcutaneous tissue upon dissection. While HAX gels were somewhat cohesive, they were much more fluid and had spread into tissue planes. The HAX-DEX gels could easily be removed (enucleated) from their capsules as a discrete entity (FIG. 39A). HAX-DEX gels were clearer than the HAX gels. These findings were in contrast to the in vitro results shown above, where HAX-DEX degraded more rapidly than HAX. All stained sections of the smears of the HAX-DEX gels showed an almost complete absence of infiltration by white cells, while all but one smear of the HAX gels had a dense collection of neutrophils and macrophages. Furthermore, the tissues and residual gel at the gel-tissue interface showed a much more vigorous cellular infiltrate in the HAX than HAX-DEX gels (FIGS. 39B-D).

Discussion

Here we have described the synthesis and characterization of a cross-linked HA hydrogel to release conjugated dexamethasone. This system has characteristics that would render it easy to apply and persist in the peritoneum. The loading of the hydrogel with dexamethasone was intentionally very low. As mentioned above, one concern was that excessive hydrophobicity of the matrix would impair the biocompatibility of the HA matrix. Furthermore, dexamethasone has very potent effects that include impaired wound healing, immunosuppression, hypertension, gastrointestinal bleeding, and others. Therefore, dose minimization was important to be able to provide local anti-inflammatory activity while minimizing systemic effects, or dehiscence of nearby wounds. Even with this very low loading, the hydrogel released clinically effective concentrations of the drug for several days, without the massive burst release that would have been caused by the dexamethasone that was dialyzed away. Conversely, the importance of these considerations regarding loading was confirmed by the mildly increased cytotoxicity of HAX-DEX compared to HAX. We note, however, that the concentrations of free HAX-DEX that obtained in vitro are unlikely to occur in vivo given the slow degradation of the cross-linked hydrogel.

Numerous investigators have reported the effectiveness of steroidal glucocorticoid receptor agonists in preventing the formation of peritoneal adhesions (Hockel et al. Prevention Of Peritoneal Adhesions In The Rat With Sustained Intraperitoneal Dexamethasone Delivered By A Novel Therapeutic System. Annales Chirurgiae Et Gynaecologiae 76 (1987) 306-313, which is incorporated herein by reference), (Buckenmaier et al. Comparison of antiadhesive treatments using an objective rat model. American Surgeon 65 (1999) 274-282, which is incorporated herein by reference), (Kucukozkan et al. Prevention of adhesions by sodium chromoglycate, dexamethasone, saline and aprotinin after pelvic surgery. ANZ J Surg 74 (2004) 1111-5, which is incorporated herein by reference). These compounds are reported to act, among other mechanisms, by reducing the production of cytokines by mesothelial cells and peritoneal macrophages. Here we studied two cytokines as measures of the effectiveness of dexamethasone release. TNF-α is an important cytokine in acute inflammation and peritoneal adhesion formation (Homdahl, L., and Ivarsson, M. L. The role of cytokines, coagulation, and fibrinolysis in peritoneal tissue repair. European Journal Of Surgery 165 (1999) 1012-1019, which is incorporated herein by reference), (Mutsaers, S. E. Mesothelial cells: Their structure, function and role in serosal repair. Respirology 7 (2002) 171-191, which is incorporated herein by reference). It stimulates mesothelial cells to secrete a variety of mediators: plasminogen activator inhibitor (PAI) (Whawell et al. Tumor Necrosis Factor-Mediated Release Of Plasminogen-Activator Inhibitor-1 By Human Peritoneal Mesothelial Cells. British Journal of Surgery 81 (1994) 214-216, which is incorporated herein by reference), (Whawell, S. A., and Thompson, J. N. Cytokine-Induced Release Of Plasminogen-Activator Inhibitor-1 By Human Mesothelial Cells. European Journal Of Surgery 161 (1995) 315-318, which is incorporated herein by reference), which slows fibrinolysis (Vanhinsbergh, V. W. M., Bauer, K. A., Kooistra, T., Kluft, C., Dooijewaard, G., Sherman, M. L., and Nieuwenhuizen, W. Progress Of Fibrinolysis During Tumor-Necrosis-Factor Infusions In Humans—Concomitant Increase In Tissue-Type Plasminogen-Activator, Plasminogen-Activator Inhibitor Type-1, And Fibrin(Ogen) Degradation Products. Blood 76 (1990) 2284-2289, which is incorporated herein by reference), (Mullarky et al. Tumor necrosis factor alpha and gamma interferon, but not hemorrhage or pathogen burden, dictate levels of protective fibrin deposition during infection. Infection And Immunity 74 (2006) 1181-1188, which is incorporated herein by referenence); IL-1, IL-6 (Mutsaers, S. E. Mesothelial cells: Their structure, function and role in serosal repair. Respirology 7 (2002) 171-191, which is incorporated herein by reference), and prostaglandins (Topley, N., Petersen, M. M., Mackenzie, R., Neubauer, A., Stylianou, E., Kaever, V., Davies, M., Coles, G. A., Jorres, A., and Williams, J. D. Human Peritoneal Mesothelial Cell Prostaglandin Synthesis—Induction Of Cyclooxygenase Messenger-Rna By Peritoneal Macrophage-Derived Cytokines. Kidney International 46 (1994) 900-909, which is incorporated herein by reference), which accelerates peritoneal inflammation; IL-8 (Mutsaers, S. E. Mesothelial cells: Their structure, function and role in serosal repair. Respirology 7 (2002) 171-191, which is incorporated herein by reference), monocyte chemoattractant protein-1 (MCP-1) (Mutsaers, S. E. Mesothelial cells: Their structure, function and role in serosal repair. Respirology 7 (2002) 171-191, which is incorporated herein by reference), and others, which induce neutrophil and monocyte recruitment. Therefore, suppression of TNF-α activity is of potential value in suppressing peritoneal adhesions. IL-6 is produced by a number of cells including macrophages, fibroblasts, and mesothelial cells. Its production is induced in mesothelial cells by IL-1 and TNF-α in a dose-dependent manner (Topley et al. Human Peritoneal Mesothelial Cells Synthesize Interleukin-6—Induction By I1-1-Beta And Tnf-Alpha. Kidney International 43 (1993) 226-233, which is incorporated herein by reference). It has numerous effects relevant to adhesion formation, including stimulating mesothelial cells to secrete PAI (Whawell et al. Cytokine-Induced Release Of Plasminogen-Activator Inhibitor-1 By Human Mesothelial Cells. European Journal Of Surgery 161 (1995) 315-318, which is incorporated herein by reference), inhibition of mesothelial cell proliferation (Lanfrancone et al. Human Peritoneal Mesothelial Cells Produce Many Cytokines (Granulocyte Colony-Stimulating Factor [Csf], Granulocyte-Monocyte-Csf, Macrophage-Csf, Interleukin-1 [I1-1], And I1-6) And Are Activated And Stimulated To Grow By I1-1. Blood 80 (1992) 2835-2842, which is incorporated herein by reference), and inducing vascular endothelial growth factor (VEGF) release, thus promoting promotes angiogenesis (Cohen et al Interleukin 6 induces the expression of vascular endothelial growth factor. Journal of Biological Chemistry 271 (1996) 736-741, which is incorporated herein by reference) like TNF-α or TGF-β.

The biocompatibility of HA-based matrices in the peritoneum, and in fact their applicability in preventing peritoneal adhesions, has been documented (Yeo et al. In situ cross-linkable hyaluronic acid hydrogels prevent post-operative abdominal adhesions in a rabbit model. Biomaterials 27 (2006) 4698-4705, which is incorporated herein by reference). Dexamethasone was selected for conjugation to the HA matrix precisely because of its potency, so that it would be possible to achieve a given inflammatory effect with the least possible alteration of the biologically benign matrix-in particular, we wished to avoid rendering it hydrophobic. The biocompatibility of the vehicle is important: one study showed that microspheres composed of the hydrophobic polymer poly-DL-lactide-co-glycolide (PLGA) with a low loading of dexamethasone worsened adhesions, whereas microspheres with a higher loading of dexamethasone decreased adhesion formation. This suggested that the effect of dexamethasone in preventing adhesions was offset by the adhesion-causing effect of the polymeric microspheres—a property that has subsequently been confirmed (Kohane et al. Biodegradable polymeric microspheres and nanospheres for drug delivery in the peritoneum. Journal of Biomedical Materials Research Part A 77A (2006) 351-361, which is incorporated herein by reference). The pre-polymer HA-DEX seemed to cause a lower cell viability than the other precursor macromolecules used here, but it is important to note, given the pharmacological effects of dexamethasone and the nature of the assay, that this could reflect an anti-proliferative effect rather than a direct toxic effect. Furthermore, it is likely that the concentrations of uncross-linked HA-DEX would be much lower than those tested here after mixing. In fact, our results show that the biocompatibility of HAX-DEX is comparable to that of HAX, and causes an even milder inflammatory response.

Conclusion

The in situ cross-linkable conjugate hydrogel of hyaluronic acid and dexamethasone had appropriate handling characteristics and released biologically effective dexamethasone, as shown in the suppression of macrophage TNF-α and IL-6 production. In vivo, the HAX-DEX gel was associated with a lesser inflammatory cell infiltrate than that from HAX gels.

### Example 15

### Prevention of Peritoneal Adhesions with an In Situ Cross-Linkable Hyaluronan Hydrogel Delivering Budesonide

Introduction

This Example describes an in situ cross-linking hyaluronic acid hydrogel (barrier device) containing the glucocorticoid receptor agonist budesonide. Budesonide was chosen because of the known role of inflammation in adhesion formation. Hyaluronic acid because of its known biocompatibility in the peritoneum. The system, which includes two cross-linkable precursor liquids, was applied using a double-barreled syringe, forming a flexible and durable hydrogel in less than 10 sec. We applied this formulation or controls to the injured sites after the second injury in a severe repeat sidewall defect-cecum abrasion model of peritoneal adhesion formation in the rabbit. Large adhesions developed in all saline-treated animals. Adhesion formation and area were slightly mitigated in animals treated with budesonide in saline or the hydrogel without hydrogel. The incidence and area of adhesions were dramatically reduced in animals treated with budesonide in the hydrogel. In subcutaneous injections in rats, we showed that budesonide in hydrogel reduced inflammation compared to hydrogel alone. In summary, budesonide in a hyaluronic acid hydrogel is convenient and highly effective in preventing adhesions in our severe repeated injury model. It is a potentially promising system for post-surgical adhesion prevention; thus, the present invention encompasses the recognition that the effectiveness of barrier devices can be greatly enhanced by concurrent drug delivery.

Peritoneal adhesions are persistent tissue connections between structures in the abdomen and pelvis, which can form following surgical trauma or infection. The incidence of post-surgical adhesions is as high as 80% and often leads to severe clinical consequences such as pain, infertility, or bowel obstruction (diZerega G S. Peritoneum, peritoneal healing, and adhesion formation. In: diZerega G S, editor. Peritoneal Surgery. New York: Springer, 2000. p. 3-37, which is incorporated herein by reference). In efforts to prevent adhesions, numerous investigators have applied pharmacological agents that intervene with critical events in adhesion formation (diZerega G S. Peritoneum, peritoneal healing, and adhesion formation. In: diZerega G S, editor. Peritoneal Surgery. New York: Springer, 2000. p. 3-37, which is incorporated herein by reference). The inflammatory component of the pathogenesis of adhesion formation has been a common target for pharmacotherapy, employing a variety of steroidal anti-inflammatory drugs (Kucukozkan T, Ersoy B, Uygur D, Gundogdu C. Prevention of adhesions by sodium chromoglycate, dexamethasone, saline and aprotinin after pelvic surgery. ANZ J Surg 2004;74(12):1111-1115; Hockel M, Ott S, Siemann U, Kissel T. Prevention Of Peritoneal Adhesions In The Rat With Sustained Intraperitoneal Dexamethasone Delivered By A Novel Therapeutic System. Annales Chirurgiae Et Gynaecologiae 1987;76:306-313; Buckenmaier C C, Pusateri A E, Harris R A, Hetz S P. Comparison of antiadhesive treatments using an objective rat model. American Surgeon 1999;65:274-282; Maurer J, Bonaventura L. The effect of aqueous progesterone on operative adhesion formation. Fertil Steril 1983;39(4):485-489; Gazzaniga A, James J, Shobe J, Oppenheim E. Prevention of peritoneal adhesions in the rat. The effects of dexamethasone, methylprednisolone, promethazine, and human fibrinolysin. Arch Surg 1975;110(4):429-432; and Jansen R. Failure of intraperitoneal adjuncts to improve the outcome of pelvic operations in young women. Am J Obstet Gynecol 1985; 153(4):363-371; all of which are incorporated herein by reference). However, the effectiveness of these agents in preventing adhesions has not been consistent in animal models (Gazzaniga A, James J, Shobe J, Oppenheim E. Prevention of peritoneal adhesions in the rat. The effects of dexamethasone, methylprednisolone, promethazine, and human fibrinolysin. Arch Surg 1975;110(4):429-432, which is incorporated herein by reference) and clinical trials (Jansen R. Failure of intraperitoneal adjuncts to improve the outcome of pelvic operations in young women. Am J Obstet Gynecol 1985;153(4):363-371, which is incorporated herein by reference), especially in preventing recurrent adhesions (Larsson B. Prevention of postoperative formation and reformation of pelvic adhesions. In: Treutner K H, Schumpelick V, editors. Peritoneal Adhesions. Berlin: Springer-Verlag Telos, 1997. p. 331-334, which is incorporated herein by reference).

Rapid clearance of drugs from the peritoneum could be a cause of the limited effectiveness of intraperitoneally applied drugs. Proper delivery systems, which would allow the drugs to maintain a high local concentration, might maximize their biological effect. In this example we have selected an in situ cross-linkable hyaluronan hydrogel (HAX) as a drug delivery system for an anti-inflammatory compound. In previous examples we have shown HAX to have excellent effectiveness in preventing peritoneal adhesions in a rabbit sidewall defect-cecum abrasion model, irrespective of the presence of nanoparticles (Yeo Y et al. In situ cross-linkable hyaluronic acid hydrogels prevent post-operative abdominal adhesions in a rabbit model. Biomaterials 2006;27:4698-4705; and Yeo Y, Ito T, Bellas E, Highley C B, Marini R, Kohane D S. In situ cross-linkable hyaluronic acid hydrogels containing polymeric nanoparticles for preventing post-operative abdominal adhesions. Ann Surg 2006:In press; both of which are incorporated herein by reference). Budesonide has potent glucocorticoid activity, comparable to that of dexamethasone (Physicians' Desk Reference: Thomson PDR, 2006, which is incorporated herein by reference), and is rapidly transformed into inactive metabolites upon systemic absorption (Physicians' Desk Reference: Thomson PDR, 2006, which is incorporated herein by reference). The anti-adhesion activity of budesonide is demonstrated in this example using a rigorous repeated injury animal model. The use of biocompatible HAX significantly improves its anti-adhesion activity, preventing adhesions completely in the majority of tested animals.

Materials and Methods

Preparation of In Situ Cross-Linkable HA Derivatives

In situ cross-linkable HA derivatives were synthesized and analyzed following a previously reported method (Yeo Y, Highley C B, Bellas E, Ito T, Marini R, Langer R, et al. In situ cross-linkable hyaluronic acid hydrogels prevent post-operative abdominal adhesions in a rabbit model. Biomaterials 2006;27:4698-4705; Bulpitt P, Aeschlimann D. New strategy for chemical modification of hyaluronic acid: preparation of functionalized derivatives and their use in the formation of novel biocompatible hydrogels. J. Biomed Mater Res 1999;47:152-169; and Jia X, Colombo G, Padera R, Langer R, Kohane D S. Prolongation of sciatic nerve blockade by in situ cross-linked hyaluronic acid. Biomaterials 2004;25(19):4797-4804; all of which are incorporated herein by reference). Briefly, HA-adipic dihydrazide (HA-ADH) was prepared by conjugating adipic dihydrazide to carboxylic groups in HA backbones, and HA-aldehyde (HA-CHO) was prepared by reacting HA with sodium periodate.

Preparation of budesonide-saline and budesonide-HAX

Budesonide was first dissolved in ethanol to make an 8.2 mg/ml stock solution. Budesonide-saline was prepared by adding 0.16 ml of the stock solution to 10 ml saline. For budesonide-HAX, 0.08 ml of the budesonide stock solution was added to 5 ml HA-ADH (20 mg/ml) and 5 ml HA-CHO (20 mg/ml), respectively. Budesonide-HAX gels were prepared by eluting the two precursor solutions though a common outlet using a Baxter double-barreled syringe. Both budesonide-saline and budesonide-HAX contained 0.13 mg/ml budesonide.

Characterization of budesonide-HAX

In situ gelation time of the budesonide-HAX was measured at room temperature as described previously (Yeo et al. In situ cross-linkable hyaluronic acid hydrogels prevent post-operative abdominal adhesions in a rabbit model. Biomaterials 2006;27:4698-4705, which is incorporated herein by reference). Briefly, 20 mg/ml HA-ADH and HA-CHO solutions were prepared in saline. Budesonide was added to each solution to 0.13 mg/ml as described above. One hundred μl of HA-ADH solution was mixed with 100 μl of HA-CHO solution under constant stirring. The gelation time was considered to be the time when the solution formed a solid globule, which separated from the bottom of the dish. Morphology of the internal structure of lyophilized budesonide-HAX was examined by scanning electron microscopy (SEM). Budesonide-HAX gel was lyophilized and fractured after cooling in liquid nitrogen. Samples were sputter-coated with palladium and gold (150 Å thick) and observed using a scanning electron microscope (JEOL JSM 6320, JEOL USA, Inc., Peabody, Mass.).

Measurement of Budesonide Solubility in Saline

The maximum solubility of budesonide in saline was determined as described (Gennaro A R, editor. Remington: The Science and Practice of Pharmacy. 20th ed. Philadelphia, Pa.: Lippincott Williams & Wilkins, 2000, which is incorporated herein by reference). First, increasing amounts of budesonide were mixed with saline to provide budesonide concentration in the system from 25 mg/ml to 0.002 mg/ml. The mixtures were constantly stirred at 37° C. for 24 hours, and then centrifuged at 12,000 rpm for 5 minutes to separate the solution phase from the solid phase. The budesonide concentrations in the solution phase were measured by High Performance Liquid Chromatography (HPLC). The saturation solubility of budesonide in saline was determined by extrapolating the line segment, of which slope was 0, to y-axis.

In a separate experiment, budesonide-saline 0.13 mg/ml was prepared as described above, divided into 1 ml aliquots, and incubated at 37° C. with constant stirring. At timed intervals, an aliquot of budesonide-saline was taken and centrifuged at 12,000 rpm for 5 minutes to separate 0.8 ml of supernatant for HPLC analysis. The remaining 0.2 ml, potentially containing precipitated budesonide, was dissolved in 0.8 ml acetonitrile and analyzed with HPLC.

In Vitro Budesonide Release Kinetics

Disk-shape budesonide-HAX was prepared in a rubber mold sandwiched between two glass slides. The diameter and thickness of the prepared hydrogel were 8 mm and 3.5 mm (˜150 μl), respectively. The budesonide-HAX gel was weighed and placed in an eppendorf tube, to which 1 ml of phosphate buffered saline (PBS) containing 10 U/ml hyaluronidase was added, and incubated at 37° C. with constant rotation. Release medium 0.5 ml was sampled after brief spin-down, and 0.5 ml of fresh medium was replaced. The release samples were frozen until HPLC analysis.

HPLC Analysis of Budesonide

The chromatographic system consisted of the HPLC solvent delivery system equipped with an automatic injector and a UV detector (1100 series, Agilent Technologies, Palo Alto, Calif.). The analytical column was an Atlantis dC18 (dC18; 4.6×250 mm; particle size 5 μm). The mobile phase was a 30:70 mixture of 0.1% acetic acid and acetonitrile, and the flow rate was 1 ml/min. A sample of 5 μl was injected onto the pre-equilibrated column followed by 10 min of wash with the mobile phase. The UV detector was set at 248 nm. A calibration curve was made by correlating the peak areas in the chromatograms and the concentrations of budesonide standards. Retention time: 6.1 min. Detection limit: 0.2 μg/ml.

In Vivo Application of Budesonide-HAX

Animals were cared for in compliance with protocols approved by the Massachusetts Institute of Technology Committee on Animal Care, in conformity with the NIH guidelines for the care and use of laboratory animals (NIH publication #85-23, revised 1985).

Subcutaneous Application of Budesonide-HAX

Male Sprague-Dawley rats (320 g-420 g) were anesthetized with 2-3% isoflurane in oxygen. One ml of budesonide-HAX or HAX was injected from a double-barreled syringe (0.5 ml of HA-ADH in one syringe, 0.5 ml of HA-CHO in the other). Budesonide-HAX was prepared as described above to contain 0.13 mg/ml budesonide. The total dose of budesonide given to the rats was therefore 0.3-0.4 mg/kg. HAX, a negative control, was prepared by adding ethanol instead of the budesonide stock solution. Budesonide-HAX or HAX was subcutaneously in the dorsal midline. Two (n=5) or 5 days (n=4) after injection, animals were sacrificed for examination of the tissue reaction. Tissues were processed for histology using standard techniques.

Preventing Peritoneal Adhesions by Budesonide-HAX

Peritoneal adhesions were induced in female albino rabbits (Oryctolagus cuniculus; New Zealand White) (3±0.5 kg) through repeated laparotomies as described in our previous study [tPA]. Briefly, cecum abrasion and excision of abdominal wall were performed to induce de novo adhesions (Yeo et al. In situ cross-linkable hyaluronic acid hydrogels prevent post-operative abdominal adhesions in a rabbit model. Biomaterials 2006;27:4698-4705, which is incorporated herein by reference). A second laparotomy was performed after 1 week to cut the adhesions and introduce additional injuries to the same locations as those injured in the first laparotomy. Excessive blood from the injury was removed, and 10 ml of budesonide-saline or budesonide-HAX was then applied to the re-abraded areas using a Baxter double-barreled syringe (n=6). Both formulations contained 1.3 mg budesonide (0.44 mg/kg). The operator was blinded as to the identity of the treatment.

Post-operative care was taken as described elsewhere (Yeo et al. In situ cross-linkable hyaluronic acid hydrogels prevent post-operative abdominal adhesions in a rabbit model. Biomaterials 2006;27:4698-4705, which is incorporated herein by reference). The animals were sacrificed 1 week after the second laparotomy by intravenous injection of sodium pentobarbital. Adhesions were scored using a modification of a reported method (Burns et al. Prevention of tissue injury and postsurgical adhesions by precoating tissues with hyaluronic acid solutions. J Surg Res 1995;59:644-652, which is incorporated herein by reference): Score 0=no adhesion; score 1=tissue adherence that would separate with gravity; score 2=tissue adhesion separable by blunt dissection; and score 3=adhesion requiring sharp dissection. If there were multiple adhesions of different scores, we chose the higher one as a representative score. Area of score 2 or 3 adhesions was measured for quantitative evaluation of the adhesions. The evaluator was blinded as to the treatment each animal received. Tissues recovered from the necropsy were fixed in 10% formalin, embedded in paraffin, sectioned, and stained with hematoxylin and eosin for histological examination.

Statistical Analysis

Data from in vivo experiments were expressed as medians with 25th and 75th percentiles since they did not always follow a normal distribution, and statistical inferences were made using Mann-Whitney U-tests following Kruskal-Wallis tests, or with Fisher's exact test, using SPSS software (Chicago, Ill.). A p-value<0.05 on a 2-tailed test was considered statistically significant.

Results

Formation of Budesonide-HAX Gels

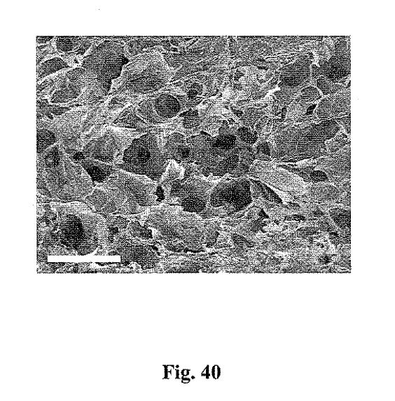

Solutions of two HA derivatives (20 mg/ml), both containing 0.13 mg/ml budesonide formed budesonide-HAX gel in 4.1±1.7 sec upon mixing with constant stirring, which was similar to the gelation time for HAX gels without budesonide (3.9±1.1 sec, p=0.72 on two-tail t-test). Under SEM, (FIG. 40), the lyophilized budesonide-HAX gel showed a porous network, typical of cross-linked hydrogels (Jia X, Burdick J A, Kobler J, Clifton R J, Rosowski J J, Zeitels S M, et al. Synthesis and Characterization of in Situ Cross-Linkable Hyaluronic Acid-Based Hydrogels with Potential Application for Vocal Fold Regeneration. Macromolecules 2004;37(9):3239-3248; and Yeo Y, Burdick J A, Highley C B, Marini R, Langer R, Kohane D S. Peritoneal application of chitosan and UV-cross-linkable chitosan. J Biomed Mater Res 2006;78A(4):668-675; both of which are incorporated herein by reference).

Solubility of Budesonide in Saline

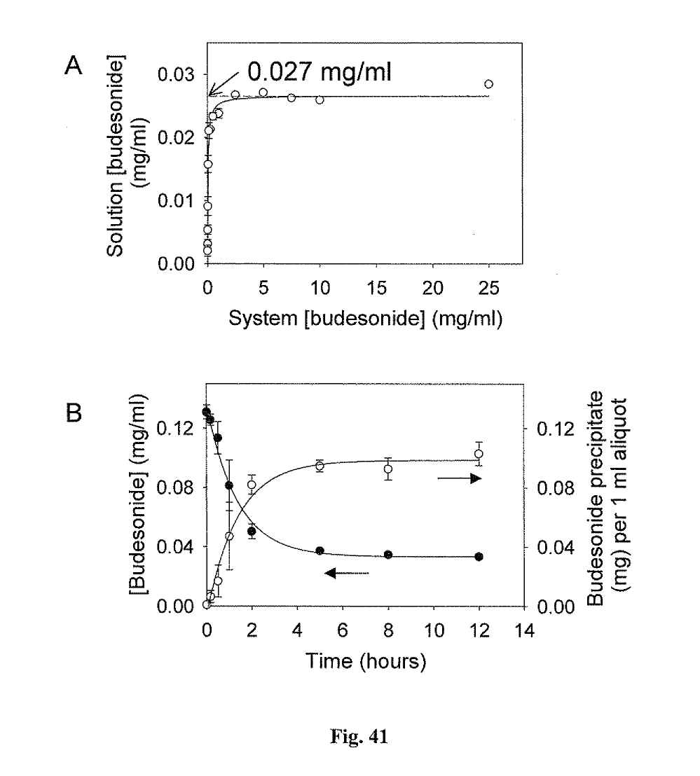

Budesonide is “practically insoluble” in water according to the United States Pharmacopoeia classification (solubility: <0.1 mg/ml) (The United States pharmacopeia. Rockville, Md.: United States Pharmacopeial Convention, Inc.). To estimate the soluble fraction of budesonide in the formulations studied here, its solubility in saline at 37° C. was measured. Varying amounts of budesonide powder were allowed to dissolve at 37° C. overnight. The budesonide concentration in the solution separated from un-dissolved solid phase reached a plateau at 0.027 mg/ml, which was defined as the saturation solubility of budesonide in saline at 37° C. (FIG. 41A).

We chose 0.44 mg/kg as the dose of budesonide for in vivo study in reference to previous experiments that used dexamethasone for adhesion prevention (Kucukozkan T, Ersoy B, Uygur D, Gundogdu C. Prevention of adhesions by sodium chromoglycate, dexamethasone, saline and aprotinin after pelvic surgery. ANZ J Surg 2004;74(12):1111-1115; Hockel M, Ott S, Siemann U, Kissel T. Prevention Of Peritoneal Adhesions In The Rat With Sustained Intraperitoneal Dexamethasone Delivered By A Novel Therapeutic System. Annales Chirurgiae Et Gynaecologiae 1987;76:306-313; and Buckenmaier C C, Pusateri A E, Harris R A, Hetz S P. Comparison of antiadhesive treatments using an objective rat model. American Surgeon 1999;65:274-282; all of which are incorporated herein by reference). In those studies, the dexamethasone dose ranged from 0.33 to 4 mg/kg. Given that the glucocorticoid potency of budesonide is comparable (systemically 40 times more potent than cortisol) to that of dexamethasone (Physicians' desk reference: Thomson PDR, 2006, which is incorporated herein by reference), the dose of budesonide used here was equivalent to those at the lower end of the range of dexamethasone doses used in other studies. To provide the dose as 10 ml budesonide-saline or budesonide-HAX to 3 kg rabbits, we prepared both budesonide-saline and budesonide-HAX to contain 0.13 mg/ml budesonide. To determine changes in solution concentration of budesonide at 37° C., budesonide-saline was incubated at 37° C., and the solution phase was analyzed over time. Immediately after preparation, budesonide-saline contained 0.13±0.004 mg/ml budesonide (=98.5±3.5% of total budesonide in the system) in the solution phase. Budesonide concentration decreased rapidly in 2 hours upon incubation at 37° C., reaching 0.034±0.001 mg/ml (=25.2±0.7% of total budesonide in the system) in 12 hours (FIG. 41B). The rest was recovered in the remaining 0.2 ml as precipitate (FIG. 41B).

In Vitro Budesonide Release Kinetics

The release kinetics of budesonide from HAX were examined in PBS containing 10 units/ml hyaluronidase, which provided an in vitro gel degradation rate comparable to that in the injured peritoneum in our previous studies (Yeo et al. In situ cross-linkable hyaluronic acid hydrogels prevent post-operative abdominal adhesions in a rabbit model. Biomaterials 2006;27:4698-4705; and Yeo Y, Ito T, Bellas E, Highley C B, Marini R, Kohane D S. In situ cross-linkable hyaluronic acid hydrogels containing polymeric nanoparticles for preventing post-operative abdominal adhesions. Ann Surg 2006:In press; both of which are incorporated herein by reference). In order for the release kinetics to reflect the release of dissolved budesonide, we incorporate an amount of drug below its saturation solubility (0.027 mg/ml).

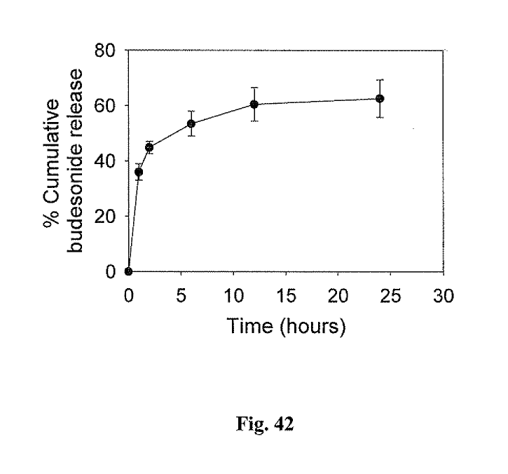

62.6±6.8% of expected budesonide was released in 24 hours (FIG. 42) from HAX gels. No significant further release followed even after the gel completely degraded. The difference between loaded and total released amounts may be explained by the loss of degrading HAX gels, which were semi-solid, during the sampling. The gel itself degraded over the next day such that only a small quantity of loose gel debris was still observed by the 8th day.

In Vivo Application of Budesonide-HAX

Preventing Peritoneal Adhesions by Budesonide-HAX

In evaluating the anti-adhesion efficacy of budesonide-saline and budesonide-HAX, we used a repeated injury model. Using a model which induced more vigorous adhesions than the conventional sidewall defect-cecum abrasion model was appealing as it would facilitate demonstrating improved anti-adhesion efficacy compared to the HAX itself, which we have already shown to be quite effective (Yeo et al. In situ cross-linkable hyaluronic acid hydrogels prevent post-operative abdominal adhesions in a rabbit model. Biomaterials 2006;27:4698-4705, which is incorporated herein by reference).

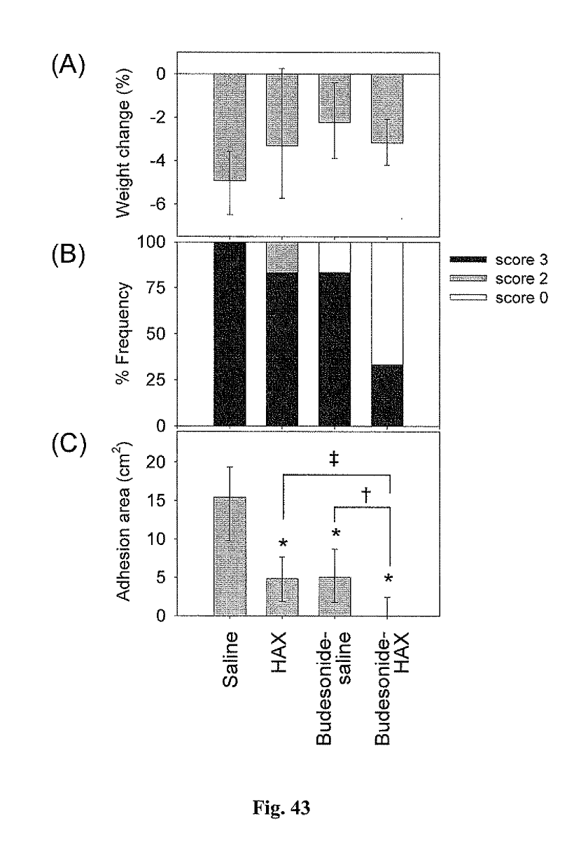

Rabbit cecum was abraded and a side-wall defect created as described above. One week later, 100% animals developed score 2 and/or 3 adhesions. Those adhesions were lysed and the sites of the cecal and side wall injuries were re-abraded. Budesonide-saline or budesonide-HAX was applied over the re-injured areas. Historical controls were provided by animals treated with saline alone (n=6) or HAX alone (n=6) from concurrent experiments. One week later, the animals were euthanized for necropsy. The two groups underwent a similar weight loss (FIG. 43A) during the survival period (p=0.378); both were comparable to that in the historical control groups treated with saline or HAX alone (n=6 each; p=0.272, Kruskal Wallis Test). Score 3 adhesions developed in 83% of animals treated with budesonide-saline (FIG. 43B). While the adhesion score was not statistically different from that of the saline-treated control, the area of adhesion was reduced approximately three-fold (p=0.01).

Budesonide in HAX markedly reduced both adhesion scores and area, even over that of budesonide-treated animals. Adhesions were completely prevented in 67% of tested animals. In the remaining animals (n=2), the adhesion areas were 1.6 and 4.6 cm², which were significantly smaller than those of saline-treated control [tPA] (p=0.003),budesonide-saline (p=0.046), and HAX [tPA] (p=0.022).

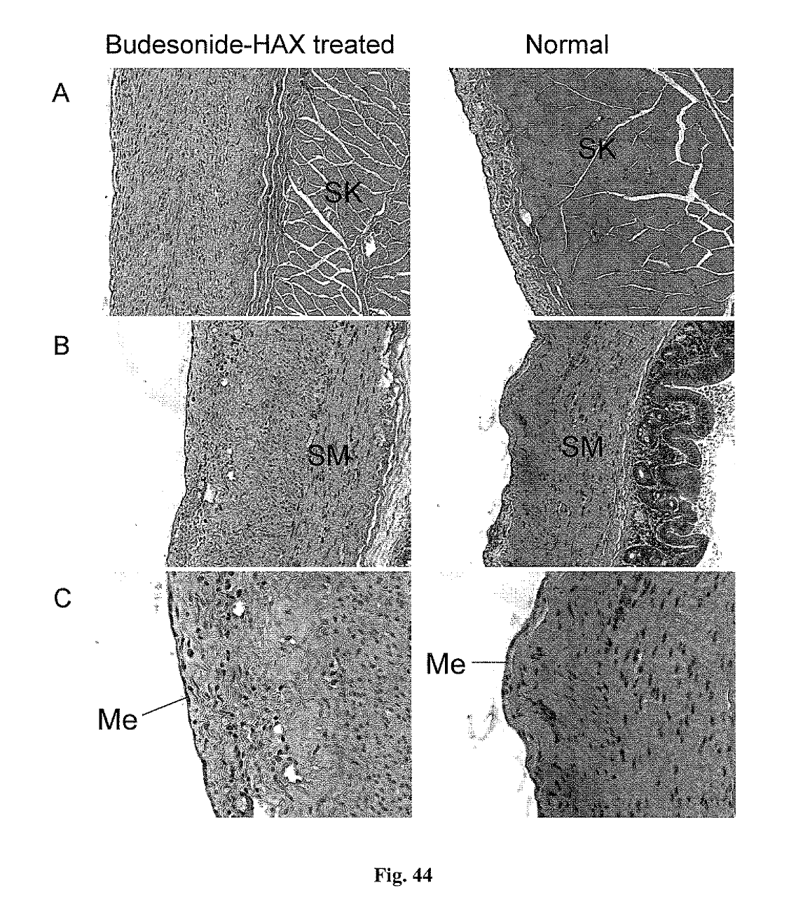

Upon histological examination, samples taken from adhesions were fibrous connective tissue connecting the smooth muscle of the cecum to the skeletal muscle layer of the abdominal wall. In both budesonide-saline and budesonide-HAX groups, we observed re-epithelization of the surfaces of healed cecum and abdominal wall (FIG. 44) comparable to that of unaffected peritoneal surfaces. There was no apparent difference in the thickness and cellular composition of the underlying granulation tissues between the two groups.

Tissue Reaction to Subcutaneous Budesonide-HAX

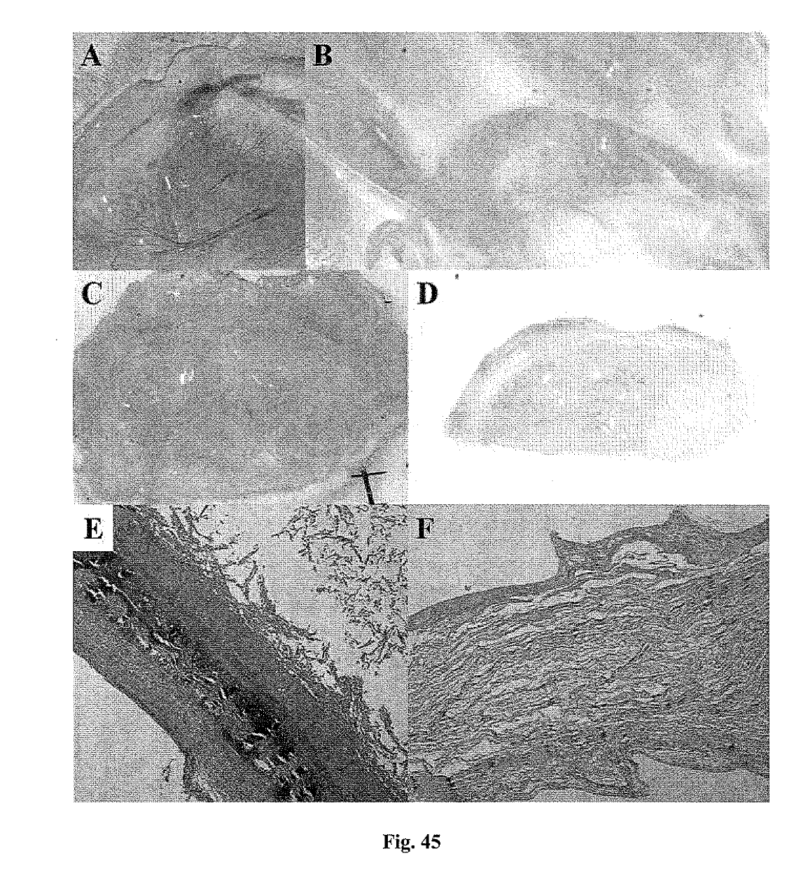

To verify the anti-inflammatory effects of budesonide-HAX, male Sprague-Dawley rats were given subcutaneous injections of HAX with or without budesonide, forming discrete bulges. Two and 5 days after injection, the gels were harvested by a blinded dissector. On gross dissection, HAX gels showed a considerable degree of inflammation (FIG. 45A), with a thick layer of hypervascularized translucent or opaque tissue obscuring the implant, which could not be dissected free of the underlying skin (FIG. 45B). In contrast, the budesonide gels showed much less vascularization and inflammation so that the clear gel could be seen (FIG. 45C), and appeared in most cases could be dissected free of the surrounding tissues (FIG. 45D). Histological assessment revealed a contrast between the two hydrogels two days after injection, such that they were easily distinguishable to a blinded observer. In samples without budesonide, the space occupied by the hydrogel was obliterated by inflammatory cells, especially neutrophils (FIG. 45E). In samples with budesonide, the inflammatory reaction was much reduced, such that the hydrogel was largely intact (FIG. 45F). At 5 days, the groups could no longer be distinguished either on gross dissection or histologically.

Discussion

Here we show that the anti-adhesion efficacy of budesonide could be significantly improved by employing a drug delivery system that could maintain a high local drug concentration at the injured surface.

Budesonide is a practically water-insoluble compound, with saturation solubility in saline of 0.027 mg/ml at 37° C. The fraction of budesonide-saline over that solubility limit quickly precipitated over 2 hours (FIG. 41B). Therefore, the budesonide-saline applied to the peritoneum was practically a mixture of a saturated solution and a suspension of budesonide precipitates. The precipitate itself would serve as a depot for continuous drug release if it were retained in the peritoneum. The budesonide-saline significantly reduced the area of adhesions as compared to the saline-treated controls, although the frequency of score 3 adhesions was not different from controls. It is possible that the effectiveness of the drug was limited by the relatively low dose, and/or that the budesonide-saline was rapidly eliminated from the peritoneum. However, the same dose was highly effective when combined with HAX.

In a rat model local sustained delivery of dexamethasone using PLGA microparticles was more effective than a dexamethasone crystal suspension in preventing adhesions (Hockel M, Ott S, Siemann U, Kissel T. Prevention Of Peritoneal Adhesions In The Rat With Sustained Intraperitoneal Dexamethasone Delivered By A Novel Therapeutic System. Annales Chirurgiae Et Gynaecologiae 1987;76:306-313, which is incorporated herein by reference), but only when a large quantity of microparticles providing a high dose of dexamethasone (4 mg/kg) was used. A smaller amount of microparticles worsened adhesions. We have previously shown that PLGA microparticles themselves induced peritoneal adhesions (Kohane D S, Tse J Y, Yeo Y, Padera R, Shubina M, Langer R. Biodegradable polymeric microspheres and nanospheres for drug delivery in the peritoneum. J Biomed Mater Res 2006;77A(2):351-361, which is incorporated herein by reference). One interpretation of these reports is that the adhesion-preventing activity of the dexamethasone was offset by the pro-adhesion effect of the vehicle. The effectiveness of the system described here may be attributable in part to the fact that the vehicle itself does not cause adhesions, and in fact has intrinsic anti-adhesion activity (Yeo et al. In situ cross-linkable hyaluronic acid hydrogels prevent post-operative abdominal adhesions in a rabbit model. Biomaterials 2006;27:4698-4705, which is incorporated herein by reference).

The subcutaneous experiments are instructive in that the anti-inflammatory effects of the budesonide gels appear to have lasted for at least two days but less than five, which equates roughly with the duration of budesonide release from the in vitro experiments. This relatively brief period of drug release therefore appears to account for the difference in outcome between the hydrogels with and without budesonide. This is congruent with the view that the critical events in adhesion formation often occur in the first 2-3 days, and suggest that the durations of drug release required for adhesion prevention may be quite short.

Many methods of adhesion prevention have failed in clinical trials despite impressive efficacy data in laboratory animals. It is possible that one contributing reason is that they were evaluated in relatively permissive animal models, where adhesion prevention is relatively easy (Wiseman D M. Animal adhesion models: design, variables, and relevance. In: diZerega G S, editor. Peritoneal Surgery. New York: Springer, 2000. p. 459-476, which is incorporated herein by reference). That was one reason we employed a more challenging repeated injury model.

Budesonide-HAX is easy to prepare and handle, effective in the presence of blood and peritoneal fluid and can be applied either via laparotomy or laparoscopy.

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. For example, it is to be understood that any of the compositions of the invention can be used for inhibiting the formation, progression, and/or recurrence of adhesions at any of the locations, and/or due to any of the causes discussed herein or known in the art. It is also to be understood that any of the compositions made according to the methods for preparing compositions disclosed herein can be used for inhibiting the formation, progression, and/or recurrence of adhesions at any of the locations, and/or due to any of the causes discussed herein or known in the art. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention (e.g., any hydrogel precursor, any polysaccharide derivative or non-polysaccharide polymer, e.g., any HA derivative or cellulose derivative, any molecular weight range, any cross-linking agent, any type of covalent bond between hydrogel precursors, any class of biologically active agent or specific agent, any particle size and/or material composition, any route or location of administration, any purpose for which a composition is administered, etc.), can be excluded from any one or more claims. For example, in certain embodiments of the invention the biologically active agent is not an anti-proliferative agent. For purposes of brevity, all of the embodiments in which one or more elements, features, purposes, or aspects is excluded are not set forth explicitly herein.

We claim:

1. A method of inhibiting adhesions, the method comprising the steps of:
administering a first hydrogel precursor to a location within the body of a subject; and
administering a second hydrogel precursor to the location within the body of the subject,
wherein the first hydrogel precursor is a cellulose derivative or a dextran derivative;
wherein the second hydrogel precursor is a dextran derivative; wherein the first and second hydrogel precursors become crosslinked to form a hydrogel following contact of the hydrogel precursors with one another; and wherein the hydrogel inhibits adhesions.

2. The method of claim 1, wherein the first hydrogel precursor comprises a first functional group; wherein the second hydrogel precursor comprises a second functional group; and wherein the first and second functional groups react with one another to form a covalent bond under physiological conditions.

3. The method of claim 1, wherein at least one of the first hydrogel precursor or the second hydrogel precursor comprises a non-polysaccharide portion.

4. The method of claim 1, wherein the cellulose derivative is selected from the group consisting of MC derivatives, CMC derivatives, and HPMC derivatives.

5. The method of claim 1, wherein the first hydrogel precursor is a CMC derivative and the second hydrogel precursor is a carboxymethyl dextran derivative.

6. The method of claim 1, wherein the second hydrogel precursor is CMDX-ADH and the first hydrogel precursor is selected from the group consisting of MC—CHO, CMC—CHO, and HPMC—CHO.

7. The method of claim 1, wherein the second hydrogel precursor is CMDX-ADH and the first hydrogel precursor is CMC—CHO.

8. The method of claim 1, wherein the first hydrogel precursor is a first dextran derivative and the second hydrogel precursor is a second dextran derivative.

9. The method of claim 8, wherein the first dextran derivative is CMDX-ADH and the second dextran derivative is CMDX—CHO.

10. The method of claim 1, wherein the hydrogel precursors are administered in solution.

11. The method of claim 1, wherein the hydrogel precursors are administered endoscopically or using a syringe.

12. The method of claim 1, wherein the hydrogel forms within between 1 and 100 seconds following contact of the hydrogel precursors with one another.

13. The method of claim 1, wherein the hydrogel precursors are administered substantially in the absence of a free crosslinking agent.

14. The method of claim 1, wherein the method comprises administering at least one solution comprising a cellulose derivative, wherein the concentration of the cellulose derivative is greater than 5 mg/ml.

15. The method of claim 1, wherein the method comprises administering at least one solution comprising a cellulose derivative, wherein the concentration of the cellulose derivative is greater than 25 mg/ml.

16. The method of claim 1, wherein the method comprises administering at least one solution comprising a cellulose derivative, wherein the concentration of the cellulose derivative is greater than 50 mg/ml.

17. The method of claim 1, wherein the method comprises administering at least one solution comprising a dextran derivative, wherein the concentration of the dextran derivative is greater than 5 mg/ml.

18. The method of claim 1, wherein the method comprises administering at least one solution comprising a dextran derivative, wherein the concentration of the dextran derivative is greater than 25 mg/ml.

19. The method of claim 1, wherein the method comprises administering at least one solution comprising a dextran derivative wherein the concentration of the dextran derivative is greater than 50 mg/ml.

20. The method of claim 1, further comprising the step of disrupting adhesions present at the location prior to administering the first and second hydrogel precursors.

21. The method of claim 1, wherein the method comprises administering a biologically active agent in solution with a hydrogel precursor or in a separate solution.

22. The method of claim 21, wherein the biologically active agent is a therapeutic agent selected from the group consisting of: anti-infective agents, anti-inflammatory agents, anti-proliferative agents, anti-neoplastic agents, anti-oxidants, angiogenesis inhibitors, immunosuppressive agents, immunomodulatory agents, anti-coagulants, proteolytic agents, agents that enhance proteolysis, free radical scavengers, anti-oxidants, inhibitors of fibrous repair, and RNAi agents.

23. The method of claim 21, wherein the biologically active agent is an anti-inflammatory agent.

24. The method of claim 23, wherein the anti-inflammatory agent is a non-steroidal anti-inflammatory agent.

25. The method of claim 24, wherein the non-steroidal anti-inflammatory agent is selected from the group consisting of celecoxib, diclofenac, diflunisal, etodolac, salicylates, fenoprofen, ibuprofen, flurbiprofen, indomethacin, ketoprofen, ketorolac, meclofamate, meclofenamate, meloxicam, naproxen, piroxicam, sulindac, salsalate, nabumetone, aspirin, oxaprozin, and tolmetin.

26. The method of claim 23, wherein the anti-inflammatory agent is a steroidal anti-inflammatory agent.

27. The method of claim 26, wherein the steroidal anti-inflammatory agent is selected from the group consisting of dexamethasone, fluorometholone, prednisolone, loteprednol, medrysone, prednisone, methylpredisolone, budesonide, cortisone, rimexolone, clobetasol, halobetasol, hydrocortisone, triamcinolone, betamethasone, fluocinolone, and fluocinonide.

28. The method of claim 1, wherein the first and second hydrogel precursors are administered at a ratio between 1:10 and 10:1 by weight.

29. The method of claim 1, wherein at least one of the hydrogel precursors has a biologically active agent covalently attached thereto.

30. The method of claim 29, wherein the biologically active agent is a therapeutic agent selected from the group consisting of: anti-infective agents, anti-inflammatory agents, anti-proliferative agents, anti-neoplastic agents, anti-oxidants, angiogenesis inhibitors, immunosuppressive agents, immunomodulatory agents, anti-coagulants, proteolytic agents, agents that enhance proteolysis, free radical scavengers, anti-oxidants, inhibitors of fibrous repair, and RNAi agents.

31. The method of claim 29, wherein the biologically active agent is an anti-inflammatory agent.

32. The method of claim 31, wherein the anti-inflammatory agent is a non-steroidal anti-inflammatory agent.

33. The method of claim 32, wherein the non-steroidal anti-inflammatory agent is selected from the group consisting of celecoxib, diclofenac, diflunisal, etodolac, salicylates, fenoprofen, ibuprofen, flurbiprofen, indomethacin, ketoprofen, ketorolac, meclofamate, meclofenamate, meloxicam, naproxen, piroxicam, sulindac, salsalate, nabumetone, aspirin, oxaprozin, and tolmetin.

34. The method of claim 31, wherein the anti-inflammatory agent is a steroidal anti-inflammatory agent.

35. The method of claim 34, wherein the steroidal anti-inflammatory agent is selected from the group consisting of dexamethasone, fluorometholone, prednisolone, loteprednol, medrysone, prednisone, methylpredisolone, budesonide, cortisone, rimexolone, clobetasol, halobetasol, hydrocortisone, triamcinolone, betamethasone, fluocinolone, and fluocinonide.

36. The method of claim 1, further comprising administering a plurality of particles together with the hydrogel precursors so that the particles become entrapped in a hydrogel formed by crosslinking of the hydrogel precursors.

37. The method of claim 36, wherein the particles are nanoparticles or microparticles that comprise a material selected from the group consisting of: poly(lactides), poly(glycolides), poly(lactide-co-glycolides), poly(lactic acids), poly(glycolic acids), poly(lactic acid-co-glycolic acids), polycaprolactone, polycarbonates, polyesteramides, poly(beta-amino esters), polyanhydrides, poly(amides), poly(amino acids), polyethylene glycol and derivatives thereof, polyorthoesters, polyacetals, polycyanoacrylates, polyetheresters, poly(dioxanones), poly(alkylene alkylates), copolymers of polyethylene glycol and polyorthoesters, biodegradable polyurethanes, and blends or copolymers of any of the foregoing polymers, and liposomes.

38. The method of claim 36, wherein the particles are present in a solution together with a hydrogel precursor prior to administration.

39. The method of claim 36, wherein the particles comprise a biologically active agent.

40. The method of claim 39, wherein the biologically active agent is a therapeutic agent selected from the group consisting of: anti-infective agents, anti-inflammatory agents, anti-proliferative agents, anti-neoplastic agents, anti-oxidants, angiogenesis inhibitors, immunosuppressive agents, immunomodulatory agents, anti-coagulants, proteolytic agents, agents that enhance proteolysis, free radical scavengers, anti-oxidants, inhibitors of fibrous repair, and RNAi agents.

41. The method of claim 39, wherein the biologically active agent is an anti-inflammatory agent.

42. A composition comprising:
(a) a cellulose derivative;
(b) a dextran derivative; and
(c) a plurality of particles.

43. The composition of claim 42, wherein the particles are nanoparticles or microparticles that comprise a material selected from the group consisting of: poly(lactides), poly(glycolides), poly(lactide-co-glycolides), poly(lactic acids), poly(glycolic acids), poly(lactic acid-co-glycolic acids), polycaprolactone, polycarbonates, polyesteramides, poly(beta-amino esters), polyanhydrides, poly(amides), poly(amino acids), polyethylene glycol and derivatives thereof, polyorthoesters, polyacetals, polycyanoacrylates, polyetheresters, poly(dioxanones), poly(alkylene alkylates), copolymers of polyethylene glycol and polyorthoesters, biodegradable polyurethanes, and blends or copolymers of any of the foregoing polymers, and liposomes.

44. The composition of claim 42, wherein the particles are biodegradable.

45. The composition of claim 42, wherein the particles comprise a biologically active agent.

46. The composition of claim 45, wherein the biologically active agent is a therapeutic agent selected from the group consisting of: anti-infective agents, anti-inflammatory agents, anti-proliferative agents, anti-neoplastic agents, anti-oxidants, angiogenesis inhibitors, immunosuppressive agents, immunomodulatory agents, anti-coagulants, proteolytic agents, agents that enhance proteolysis, free radical scavengers, anti-oxidants, inhibitors of fibrous repair, and RNAi agents.

47. The composition of claim 45, wherein the biologically active agent is an anti-inflammatory agent.

48. The composition of claim 42, wherein the composition is a hydrogel in which the cellulose derivative and the dextran derivative are crosslinked to one another.

49. The composition of claim 42 wherein the cellulose derivative or the dextran derivative has a biologically active agent covalently attached thereto.

50. The composition of claim 49, wherein the biologically active agent is a therapeutic agent selected from the group consisting of: anti-infective agents, anti-inflammatory agents, anti-proliferative agents, anti-neoplastic agents, anti-oxidants, angiogenesis inhibitors, immunosuppressive agents, immunomodulatory agents, anti-coagulants, proteolytic agents, agents that enhance proteolysis, free radical scavengers, anti-oxidants, inhibitors of fibrous repair, and RNAi agents.

51. A method of administering particles to a location within the body comprising:
administering a composition comprising particles, a first hydrogel precursor, and a second hydrogel precursor, to the location,
wherein the first hydrogel precursor is a cellulose derivative or a dextran derivative;
wherein the second hydrogel precursor is a dextran derivative; and wherein the first and second hydrogel precursors form a hydrogel that entraps the particles therein following administration.

52. The method of claim 51, wherein the composition is administered as one or more solutions at least one of which contains particles.

53. The method of claim 51, wherein the particles comprise a biologically active agent.

54. The method of claim 51, wherein the particles are nanoparticles or microparticles that comprise a material selected from the group consisting of: poly(lactides), poly(glycolides), poly(lactide-co-glycolides), poly(lactic acids), poly(glycolic acids), poly(lactic acid-co-glycolic acids), polycaprolactone, polycarbonates, polyesteramides, poly(beta-amino estera), polyanhydrides, poly(amides), poly(amino acids), polyethylene glycol and derivatives thereof, polyorthoesters, polyacetals, polycyanoacrylates, polyetheresters, poly(dioxanones), poly(alkylene alkylates), copolymers of polyethylene glycol and polyorthoesters, biodegradable polyurethanes, and blends or copolymers of any of the foregoing polymers, and liposomes.

55. A method of administering a biologically active agent to a subject comprising steps of:
administering a composition comprising a biologically active agent, a first hydrogel precursor, and a second hydrogel precursor to the location,
wherein the first hydrogel precursor is a cellulose derivative or a dextran derivative;
wherein the second hydrogel precursor is a dextran derivative; and wherein the first and second hydrogel precursors form a hydrogel that entraps the biologically active agent therein.

56. The method of claim 55, wherein the biologically active agent is covalently attached to a hydrogel precursor.

57. The method of claim 55, wherein the biologically active agent is a therapeutic agent selected from the group consisting of: anti-infective agents, anti-inflammatory agents, anti-proliferative agents, anti-neoplastic agents, anti-oxidants, angiogenesis inhibitors, immunosuppressive agents, immunomodulatory agents, anti-coagulants, proteolytic agents, agents that enhance proteolysis, free radical scavengers, anti-oxidants, inhibitors of fibrous repair, and RNAi agents.

58. The method of claim 55, wherein the biologically active agent is an anti-inflammatory agent.

59. The method of claim 55, wherein the biologically active agent is physically associated with particles.

60. The method of claim 55, wherein the biologically active agent is physically associated with nanoparticles or microparticles that comprise poly(lactides), poly(glycolides), poly(lactide-co-glycolides), poly(lactic acids), poly(glycolic acids), poly(lactic acid-co-glycolic acids), polycaprolactone, polycarbonates, polyesteramides, poly(beta-amino esters), polyanhydrides, poly(amides), poly(amino acids), polyethylene glycol and derivatives thereof, polyorthoesters, polyacetals, polycyanoacrylates, polyetheresters, poly(dioxanones), poly(alkylene alkylates), copolymers of polyethylene glycol and polyorthoesters, biodegradable polyurethanes, and blends or copolymers of any of the foregoing polymers, and liposomes.
